(19) United States
(12) Patent Application Publication
(10) Pub. No.: US 2003/0180302 A1
(43) Pub. Date: Sep. 25, 2003

(54) Use of polypeptides or nucleic acids for the diagnosis or treatment of skin disorders and wound healing and for the identification of pharmacologically active substances

(76) Inventors: Eckhard Wolf, Oberschleissheim (DE); Sabine Werner, Zurich (CH); Jorn-Peter Halle, Penzberg (DE); Johannes Regenbogen, Martinsried (DE); Andreas Goppelt, Munchen (DE)

Correspondence Address: CLARK & ELBING LLP, 101 FEDERAL STREET, BOSTON, MA, 02110, US

(21) Appl. No.: 10/376,564

(22) Filed: Feb. 28, 2003

Related U.S. Application Data
(63) Continuation-in-part of application No. 09/886,319, filed on Jun. 20, 2001, now Pat. No. 6,586,185.
(60) Provisional application No. 60/222,081, filed on Aug. 1, 2000.

(30) Foreign Application Priority Data
Jun. 20, 2000 (DE) DE 10030149.5

(51) Int. Cl. C12Q 1/68; G01N 33/53; G01N 33/567; A61K 38/17; A61K 48/00; A61K 39/395

(52) U.S. Cl. 424/146.1; 435/6; 435/7.2; 514/12; 514/44

(57) ABSTRACT

Methods of promoting wound healing or treating a wound healing disorder in a patient and methods of preventing or treating a skin disorder in a patient are disclosed. As an active ingredient Eps 8 is disclosed.

This application is a continuation-in-part application of U.S. 09/886,319 filed on Jun. 20, 2001, which claims benefit of the filing date of the U.S. provisional application No. 60/222,081 filed Aug. 1, 2000, which claims priority from foreign patent application DE 10030149.5, filed Jun. 20, 2000, in Germany, all hereby incorporated by reference.

The invention relates to the use of polypeptides selected from SEQ ID No. 1 to SEQ ID No. 26 and/or SEQ ID No. 29 to SEQ ID No. 48 and/or SEQ ID No. 55 to SEQ ID No. 58 and/or SEQ ID No. 63 to SEQ ID No. 73 and/or SEQ ID No. 80 to SEQ ID No. 82 and/or of a nucleic acids encoding these, and/or of a cell expressing said polypeptide or said nucleic acid, for the diagnosis, prevention and/or treatment of disorders, in particular skin disorders, wound healing, and/or wound healing disorders, and/or for the identification of pharmacologically active substances.

Wounds in general heal without therapeutic intervention. However, there are numerous disorders in which wound healing plays a role, such as, for example, diabetes mellitus, arterial occlusive diseases, psoriasis, Crohn's disease, epidermolysis bullosa, age-related skin changes or innervation disorders. Wound healing disorders lead to a delayed healing of wounds or to chronic wounds. These disorders can be caused by the nature of the wound (e.g. large-area wounds, deep and mechanically expanded operation wounds, burns, trauma, decubitus), medicinal treatment of the patients (e.g. with corticoids) but also by the nature of the disorder itself. For example, 25% of the patients with Type II diabetes thus frequently suffer from chronic ulcers (“diabetic foot”), of which approximately half necessitate expensive hospitalized treatments and nevertheless finally heal poorly. Diabetic foot causes more stays in hospital than any other complication associated with diabetes. The number of these cases in diabetes Type I and II is on the increase and represents 2.5% of all hospital admissions. Moreover, wounds heal more poorly with increasing age of the patients. An acceleration of the natural wound healing process is often desirable as well in order to decrease, for example, the danger of bacterial infections or the rest periods of the patients.

Further disorders can also occur after successful wound closure. While foetal skin wounds heal without scar formation, after injuries in the postnatal period formation of scars always occurs, which often represent a great cosmetic problem. In the case of patients with large-area burn wounds, the quality of life can moreover be dramatically adversely affected, especially as in scarred skin the appendages, such as hair follicles, sweat and sebaceous glands are missing. In the case of appropriate genetic disposition, keloids can also occur, hypertrophic scars which proliferate into the surrounding skin.

The process of skin healing requires complex actions and interactions of various cell types which proceed in a coordinated manner. In the wound healing process, the following steps are differentiated: blood clotting in the area of the wound, the recruitment of inflammatory cells, reepithelialization, the formation of granular tissue and matrix remodeling. Little is known up to now about the exact reaction pattern of the cell types involved during the phases of proliferation, migration, matrix synthesis and contraction, just like about the regulation of genes such as, for example, growth factors, receptors and matrix proteins.

Thus until now only a few satisfactory therapies have been developed in order to treat wound healing disorders. Established forms of therapy are restricted to physical assistance of wound healing (e.g. dressings, compresses, gels) or the transplantation of skin tissues, cultured skin cells and/or matrix proteins. In recent years, growth factors have been tested for improving wound healing without, however, improving the conventional therapy decisively. The diagnosis of wound healing disorders is also based on not very meaningful optical analyses of the skin, since a deeper understanding of the gene regulation during wound healing was lacking until now.

Not very satisfactory therapies have been developed until now for other disorders of regenerative processes as well. Here too, the knowledge of gene regulation is advantageous for the development of diagnostics and therapies. It has been shown (Finch et al., 1997, Am. J. Pathol. 151: 1619-28; Werner, 1998, Cytokine Growth Factor Rev. 9: 153-165) that genes relevant to wound healing also play a crucial role in dermatological disorders which are based on disorders of the regeneration of the skin, and generally in regenerative processes. Thus the growth factor KGF not only plays a crucial role in the regulation of the proliferation and differentiation of keratinocytes during wound healing, but is also an important factor in the hyperproliferation of the keratinocytes in psoriasis and regenerative processes in the intestine (in Crohn's disease and ulcerative colitis).

It is therefore the object of the present invention to make available polypeptides and nucleic acids encoding these which are involved in processes in disorders of skin cells, in wound healing and/or in wound healing disorders, and whose use decisively improves the diagnosis, prevention and/or treatment, and also the identification and development of pharmaceuticals which are effective in connection with these disorders.

Diseases of the skin, wound healing and its pathological disorders within the meaning of the invention are to be discriminated from skin diseases which are accompanied by uncontrolled cell proliferation and cell differentiation, in particular by skin cancer. In the latter disease, transformation of individual cells occurs, which therefore begin to proliferate in an uncontrolled, autonomous manner, i.e. isolated from interactions with other cell types, and at the same time transmit the pathological changes to the daughter cells. It is thus a disorder which is accompanied by a loss of interactions, for example of cell-cell adhesion and of typical cell properties. In contrast, diseases within the meaning of the invention are based on disorders of cell-cell interactions. The formation of skin diseases within the meaning of the invention is caused by a large number of factors. Thus in the case of psoriasis, for example, genetic predispositions and malfunctions of the T cells, fibroblasts and keratinocytes probably both play an important role (see, for example, Nair et al., 1997; Hum. Molec. Genet. 6: 1349-1356; Gottlieb et al., 1995, Nat. Med. 1: 442-447; Saiag et al., 1985, Science, 230: 669-672; Pittelkow, 1998, in Roenigk 1998: 225-246). The course of wound healing can also be modulated by various endogenous and exogenous factors. Even small perturbations of the interactions between the different cell types of the dermis and epidermis themselves, but also of interactions with other tissues and organs such as the blood vessel system, the nervous system and the connective tissue, can lead to disturbed wound healing followed by scar formation. Furthermore, infections, aging, disorders such as diabetes and immune disorders and also vitamin deficiencies can adversely affect the wound-healing process. Similarly complex interactions are also described for other skin diseases such as vitiligo and atopic dermatitis. This essentially differentiates the skin diseases from cancer of these organs. Preferred examples of skin diseases encompass psoriasis, eczema, especially atopic eczema and disorders of pigmentation of the skin, especially vitiligo. Examples of disorders of wound healing are wounds of patients suffering from diabetes or alcoholism, wounds infected with microorganisms, ischemic wounds and wounds of patients with impaired circulation or venous stasis. Especially preferred examples of badly healing wounds are diabetic, neuropathic, venous and arterial ulcers, especially diabetic ulcers.

The autonomous character of carcinomatous disorders is also seen at the therapeutic level. In the case of non-metastasizing tumors, cancer can be treated surgically. This physical treatment is possible, as no interactions take place between tumor cells and the surrounding cells and tissues, so that the patient can be cured by simple excision of the tumor, whereas this is not possible in the case of skin diseases within the meaning of the invention—the pathological disorders of the cell-cell and/or tissue-tissue interactions cannot be abolished by excision of affected skin sites. The fact that the diseases compared are diseases which are based on a fundamentally different mechanism becomes clear if the therapeutic approaches are compared. In cancer disorders and diseases which are accompanied by uncontrolled cell proliferation, therapy is directed at the destruction of rapidly growing cells, e.g. by means of cytostatics. These toxic substances prevent the growth of actively proliferating cells while cells in the G0 phase of the cell cycle are not affected. In contrast, the treatment of disorders of skin cells within the meaning of the invention aims at the modulation of the interactions between the different cell types, for example by affecting the migration, proliferation and differentiation of individual cell types. Skin diseases within the meaning of the invention cannot be cured by general inactivation of proliferating cells. The methodological approach to the identification of the nucleic acids used according to the invention, which are involved in wound healing and/or in processes of the skin diseases within the meaning of the invention, differs clearly from procedures which are suitable for identifying nucleic acids which are involved in processes of the carcinomatous disorders. The latter can be identified by analysis of genes of the cell type affected by cancer which are expressed in differential form. The aim of the assay of the present invention is, however, rather to identify, by comparison of the expression in diseased and healthy tissue biopsies, genes which are involved in the complex processes of the skin diseases and/or in wound healing and/or its pathological disorders. This procedure would be unsuitable for the identification of genes relevant to cancer.

In the analysis of gene expression during the wound healing process it was surprisingly possible to identify genes, that until now were not connected with diagnosis, prevention and/or treatment of disorders, of wound healing and/or of disorders of wound healing, and for the identification of pharmacologically active substances but whose regulation is essential for the healing process and which are thus in a causal relationship with diagnosis, prevention and/or treatment of disorders, disorders of the skin, in wound healing and/or disorders of wound healing, and for the identification of pharmacologically active substances. The polypeptides of these genes do not belong to the targets known until now for diagnosis—such as, for example, the indication—and/or the treatment—such as, for example, the modulation—of disorders or wound healing, of disorders of wound healing or for the identification of pharmacologically active substances, such that completely novel therapeutic approaches result from this invention.

The object is therefore achieved according to the invention by the use of one or more polypeptides selected from a sequence of SEQ ID No. 55 to SEQ ID No. 58 or functional variants thereof and/or of a nucleic acid or a variant thereof encoding these, and/or of a cell expressing said polypeptide or a functional variant thereof or said nucleic acid or a variant thereof, if appropriate combined or together with suitable additives and/or auxiliaries, for the diagnosis, treatment and/or prevention of diseases, in particular diseases of skin cells, of wound healing and/or its pathological disorders, and/or its use for the identification of pharmacologically active substances.

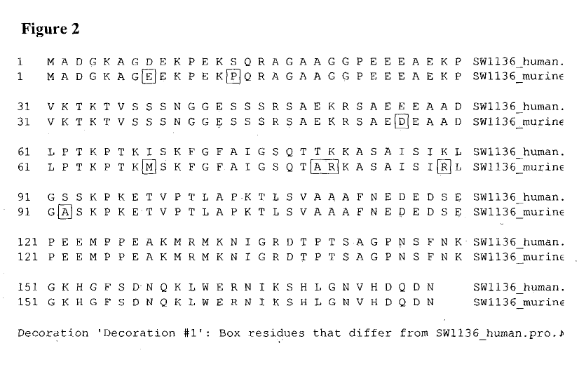

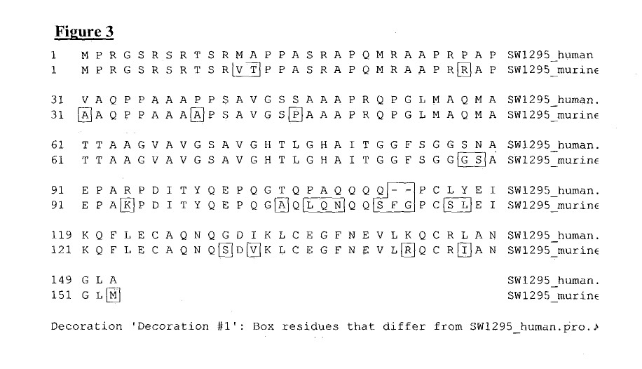

The exact biological functions of the polypeptides selected from a sequence of SEQ ID No. 55 to SEQ ID No. 58 used according to the invention are unknown. In the investigations in the context of this invention, it was possible for the first time to determine a relationship of the polypeptides according to the invention with disorders, for example skin diseases. The accession numbers of the polypeptide sequences according to the invention and their cDNAs, if known, are listed in Table 3. The cDNA sequences of the polypeptides of SEQ ID No. 55 to 57 are listed under SEQ ID No. 50 to 52. FIG. 2 and FIG. 3 show the comparison of human and murine polypeptide sequences.

In the analysis of gene expression during the wound-healing process, it was possible to identify further genes whose already known and described functions have previously not been connected with skin diseases or wound healing, for example with disturbed wound healing, but whose regulation is essential for the wound-healing process and which have thus been brought for the first time into causal relationship with skin diseases, for example with disturbed wound healing. The polypeptides of these genes do not belong to the previously known targets of skin disease therapies and/or wound healing or its disorders, so that completely new therapeutic approaches result from this invention.

The object of the invention is furthermore achieved by the use of at least one polypeptide selected from a sequence of SEQ ID No. 1 to SEQ ID No. 20 and/or SEQ ID No. 31 to SEQ ID No. 48 and/or SEQ ID No. 63 to SEQ ID No. 70 and/or SEQ ID No. 80 to SEQ ID No. 82 or functional variants thereof and/or nucleic acids or variants encoding these, and/or of a cell expressing said polypeptide or a functional variant thereof or said nucleic acid or variants thereof, if appropriate combined or together with suitable additives and/or auxiliaries, for the diagnosis, prevention and/or treatment of diseases of skin cells, of wound healing and/or its pathological disorders, and/or its use for the identification of pharmacologically active substances.

The following polypeptides can be used according to the invention:

The tumor susceptibility gene TSG 101 from mouse (SEQ ID No. 1) or human (SEQ ID No. 2) that is known from WO 97/18333 and U.S. Pat. No. 5,892,016 (Li and Cohen, 1996, Cell 85:319-329; Li et al., 1997, Cell 88:143-154). The functional inactivation of TSG 101 in fibroblasts leads to cellular transformation and to the ability to form metastasizing tumors. TSG 101-deficient neoplastic cells show abnormalities in mitosis associated processes (Xie et al., 1998, Proc. Natl. Acad. Sci. U.S.A. 95:1595-1600). Furthermore, a role as transcriptional modulator is assumed (Sun et al., 1999, Cancer 86:689-696). In addition to the human polypeptide according to SEQ ID No. 2, the splice variant according to SEQ ID No. 82 (SWISSProt: Q99816) can also be used.

The tumor suppressor protein MASPIN, that is known from U.S. Pat. No. 5,905,023, U.S. Pat. No. 5,801,001, U.S. Pat. No. 5,470,970 and WO 94/05804 from mouse (SEQ ID No. 3) or human (SEQ ID No. 4) (Zou et al., 1994, Science, 263, 526-529). MASPIN is a serine protease inhibitor (Zhang et al., 1997, Mol. Med. 3:49-59) that is expressed in normal breast and prostate epithelial cells (Zhang et al., 1997, Proc. Natl. Acad. Sci. U.S.A. 94:5673-5678) and plays an essential role in the development of the breast gland (Zhang et al., 1999, Dev. Biol. 215:278-287).

The RNA-polymerase I termination factor TTF-I from mouse (SEQ ID No. 5) or human (SEQ ID No. 6) (Evers and Grummt, 1995, Proc. Natl. Acad. Sci. U.S.A. 92:5827-5831). The protein mediates the termination of transcription of nbosomal genes (Kuhn et al., 1990, Nature 344:559-62) as well as the transcriptional activation of ribosomal genes in chromatin (Langst et al., 1998, EMBO J. 17:3135-45).

The protooncogen B-raf from mouse (SEQ ID No. 7) or human (SEQ ID No. 8), that is known from WO 91/02077 and U.S. Pat. No. 7,745,381 (Miki et al., 1991, Proc. Natl. Acad. Sci. U.S.A. 88:5167-5171; Stephens et al., 1992, Mol. Cell. Biol. 12:3733-3742). The B-raf protooncogene belongs to the Raf-family comprising serine/threonine protein kinases that link the stimulation of growth factor receptors and the activation of mitogen-activated protein kinases (Mason et al., 1999, EMBO J 18:2137-48). Furthermore, B-Raf can inhibit apoptosis (Erhardt et al., 1999, Mol. Cell. Biol. 19:5308-15).

Prothymosin alpha from mouse (SEQ ID No. 9) or human (SEQ ID No. 10), that is known from U.S. Pat. No. 4,716,148 and U.S. Pat. No. 4,659,694 (Schmidt and Werner, 1991, Biochim. Biophys. Acta. 1088:442-444; Eschenfeldt and Berger, 1986, Proc. Natl. Acad. Sci. U.S.A. 83:9403-9407). It codes for a small acidic nuclear protein that has a role in the proliferation of cells (Tao et al., 1999, J. Cell Physiol. 178:154-63).

The GOGLI 4-TRANSMEMBRANE SPANNING TRANSPORTER or MTP (mouse transporter protein) from mouse (SEQ ID No. 11) or human (SEQ ID No. 12) (Hogue et al., 1996, J. Biol. Chem. 271:9801-9808; Nagase et al., 1995, DNA Res. 2:37-43). It is a strongly conserved membrane protein that is localized in lysosomes and endosomes of mammalian cells. The protein is responsible for the subcellular distribution of a number of different small hydrophobic molecules and contributes to the sensitivity respectively resistance of mammalian cells towards particular active substances (Hogue et al., 1999, J. Biol. Chem. 274:12877-82). For the mouse homologue, an alternative polypeptide truncated at the C terminus by 89 amino acids is formed by an alternative translation initiation site (see SwissProt: Q60961). This murine polypeptide can also be used according to the invention.

CCR-1 from mouse (SEQ ID No. 13) or human (SEQ ID No. 14) (Post et al., 1995, J. Immunol. 155:5299-5305) that is an eosinophilic receptor for the CC-chemokin eotaxin (Gao et al., 1996, Biochem. Biophys. Res. Comm. 223:679-84). CCR-1 is expressed in heart, spleen and lung (Gao and Murphy, 1995, Genomics 29:294-96).

The nucleosome binding protein HMG-14 from mouse (SEQ ID No. 15) or human (SEQ ID No. 16) (Landsman and Bustin, 1990, Nucleic Acids Res. 18:5311; Landsman et al., 1986, J. Biol.,Chem. 261:16082-16086), that opens up higher order chromatin structures and thus increases the transcription and replication potential of chromatin (Herrera et al., 1999, Mol. Cell. Biol. 19:3466-73). Split hand/foot deleted I from mouse (SEQ ID No. 17) or human (SEQ ID No. 18), that is a candidate gene for the autosomal dominant form of “split hand/split foot malformation disorder” that is expressed in limb buds, in the “cranofacial primordia” and in the skin (Crackower et al., 1996, Hum. Mol. Genet. 5:571-9).

The orphan receptor TAK1 or TR4 from mouse (SEQ ID No. 19) or human (SEQ ID No. 20) (Hirose et al., 1995, Gene 163:239-242; Hirose et al., 1994, Mol. Endocrinol. 8:1667-1680), that belongs to the superfamily of nuclear hormone receptors (Hirose et al., 1994, Mol, Endocrinol. 8:1667-80). As a homodimer, TR4 influences the multitude of signal transduction pathways, among them retinoic acids, thyroid hormone, vitamin D3 and “ciliary neutrophic factor”. Additionally TR4 forms heterodimers with the androgen receptor (Lee et al., 1999, Proc. Natl. Acad. Sci. U.S.A. 96:14724-9).

BAF57 from mouse (SEQ ID No. 31) or human (SEQ ID No. 32), that is known from WO 95/14772, which is a part of the chromatin remodeling SWI/SNF complex of higher eukaryotes (Wang et al., 1998, Proc. Natl. Acad. Sci. U.S.A. 95:492-498.) The SWI/SNF complexes regulate the transcription of specific genes by relieving chromatin mediated repression of transcription (Wolffe and Guschin, 2000, J. Struct. Biol. 129:102-122). Additionally, a role has been shown for the switch from expression of fetal to adult globin in mice (Armstrong et al., 1999, Proc. Natl. Acad. Sci. U.S.A. 96:349-54). In addition to the known human and mouse polypeptides, the closely related novel human polypeptide having a significantly different sequence (SEQ ID No. 80) can also be used. The cDNA encoding the polypeptide according to SEQ ID No. 80 is indicated under SEQ ID No. 83.

Epidermal growth factor receptor kinase substrate EPS 8 from mouse (SEQ ID No. 33) or human (SEQ ID No. 34), that is known from U.S. Pat. No. 7,935,311, which amplifies the EGF dependent mitogenic signales (Wong et al., 1994, Oncogene 9:3057-3061; Fazioli et al., 1993, EMBO J. 12:3799-3808). Both overexpression as well as constitutive phosphorylation of EPS 8 has been described in connection with tumor development (Matoskova et al., 1995, Mol. Cell Biol. 15:3805-3812).

KIAA1247 from human (SEQ ID No. 36), that according to WO 99/34004 can be applied as a marker protein for cancer metastasis. Additionally, a KIAA1247 homologue from rat is known as protein from WO 98/53071, whose expression is induced in injured or regenerating tissue, in particular from kidney tissue of the rat. In addition to the known polypeptide from human, the polypeptide from mouse (SEQ ID No. 35) that is mentioned for the first time in this work can also be used. In addition two human splice variants of the gene encoding the polypeptide of SEQ ID No. 36, which are mentioned for the first time in this work, can be used according to the invention. These splice variants encode shorter variants of SEQ ID No. 36: the amino acids 652 to 654 and 664 to 681 or the amino acids 664 to 681 of the polypeptide of SEQ ID No. 36, respectively, are deleted in these variants. In addition, other KIAA1247 polypeptides can be used according to the invention, which result from alternative translation initiation ATG-codon. Examples of such variants are disclosed in WO 00/73454 and in WO 00/58473.

Phospholipase inhibitor GIPL from human (SEQ ID No. 38), that is known from U.S. Pat. No. 5,948,626, U.S. Pat. No. 5,663,059 and U.S. Pat. No. 5,811,520. In addition to the already known polypeptide from human the polypeptide from mouse (SEQ ID No. 37), that is mentioned in this work for the first time, and the closely related polypeptides with a significantly divergent sequence (SEQ ID No. 45 and SEQ ID No. 81), which are mentioned in this work for the first time, can be used. The cDNA encoding the polypeptide according to SEQ ID No. 81 is indicated under SEQ ID No. 84.

EAT/MCL-1 from mouse (SEQ ID No. 39) or human (SEQ ID No. 40), that is known from WO 95/28497, which is expressed in numerous tissues (Krajewski et al., 1995, Am. J. Pathol. 146:1309-19) and that plays role in cutaneous malignant melanoma (Tang et al., 1998, Clin. Cancer Res. 4:1865-71).

TSC-22 (TGF-beta-stimulated clone 22 gene) from human (SEQ ID No. 42) and mouse (SEQ ID No. 41) (Jay et al., 1996, Biochem. Biophys. Res. Commun. 222:821-826; Shibanuma et al., 1992, J. Biol. Chem. 267:10219-10224), that belongs to the “leucine-zipper” family of transcription factors (Kester et al., 1999, J. Biol. Chem. 274:27439-47). Transcription of TSC-22 is induced by variety of stimuli as, for instance, growth inhibitors (Kester et al., 1999, J. Biol. Chem. 274:27439-47). Additionally an increased expression of TSC-22 during development of the mouse embryo was observed at locations where mesenchymal-epithelial interaction occurs (Dohrmann et al., 1999, Mech. Dev. 84:147-51).

Gamma-sarcoglycan from human (SEQ ID No. 44) or mouse (SEQ ID No. 43) (Noguchi et al., 1995, Science 270:819-822; Noguchi et al., 1999, Biochem. Biophy. Res. Commun. 262:88-93), that is known from JP 100 57 065 and U.S. Pat. No. 5,837,537. Gamma-sarcoglycan is a component of the sarcoglycan complex that again is a subcomplex of the dystrophin gycoprotein complex. This establishes a connection between the extracellular matrix and the actin cytoskeleton (Hack et al., 2000, Microsc. Res. Tech. 48:167-80). Mutation of gamma-sarcoglycan has been described as a primary genetic defect of a muscular dystrophy (SCARMD) (Noguchi et al., 1995, Science 270:819-822).

Cysteine proteinase inhibitor cystatin C from human (SEQ ID No. 47) or mouse (SEQ ID No. 46) (Abrahamson et al., 1987, FEBS Lett. 216:229-233; Solem et al., 1990, Biochem. Biophys. Res. Commun. 172:945-951), that is known from WO 99/38882, WO 88/09384, DE 372 4 581, JP 012 02 287, JP 010 74 988 and U.S. Pat. No. 5,212,297. Cystein protease inhibitors play a role in inflammatory disorders as, for example, rheumatism (Lenarcic et al., 1988, Biol. Chem. Hoppe Seyler 369 (Suppl.):257-261) and in vascular disorders (Shi et al., 1999, J. Clin. Invest. 104:1191-1197). In addition to the known polypeptide variant from mouse (SEQ ID No. 46) (Solem et al., 1990, Biochem. Biophys. Res. Commun. 172:945-951) the closely related polypeptide with a divergent sequence, that has been described in this work for the first time (SEQ ID No. 48) can also be used.

The tyrosine kinase Fer from mouse (SEQ ID No. 63) or human (SEQ ID No. 64) (SwissProt: P70451; Hao et al., 1989, Mol. Cell. Biol. 9:1587-1593), that is both localized in the nucleus as well as in the cytoplasm (Hao et al., 1991, Mol. Cell. Biol. 11:1180-1183). A role for Fer has been postulated both for cell-cell-adhesion (Rosato et al., 1998, Mol. Cell. Biol. 18:5762-5770) as well as a role as proto-oncogene (Morris et al., 1990, Cytogenet. Cell. Genet. 53:196-200).

The C-C cytokine MRP-3 (macrophage inflammatory protein 3) from mouse (SEQ ID No. 65) or human (SEQ ID No. 66), that is known from WO 99/28473, WO 96/34891 and WO 98/14582, that is also called C10, MPIF-1 (Myeloid Progenitor Inhibitory Factor-l), CK-beta-8 or small inducible cytokine A23 (Orlosfsky et al., 1991, Cell Regul. 2:403-412; Li and Ruben, 1996, U.S. Pat. No. 5,504,003). A high expression of MRP-3 a macrophages has been observed in chronic infection of the peritoneum (Wu et al., 1999, Cytokine 11:523-30). As a typical C-C cytokine MRP-3 is a chemoattractant for leukocytes (Haelens et al., 1996, Immunobiology 195:499-521) but it also effects osteoclasts (Votta et al., 2000, J. Cell Physiol. 183:196-207). In addition it has been observed that MRP-3 mRNA is not significantly upregulated by stimuli that are connected to wound healing (Orlofsky et al., Cell Regul., 1991, 2:403-412).

The nicotinamide N-methyltransferase NNMT from mouse (SEQ ID No. 67) or human (SEQ ID No. 68) (Aksoy et al., 1994, J. Biol. Chem. 269:14835-14840; Yan et al., 1997, Biochem. Pharmacol. 54:1139-1149), that catalyzes the methyltransfer of S-adenosylmethionine to nicotineamide. There are several pieces of evidence, that NNMT can regulate the growth of liver cells (Seifert et al., 1984, Biochim, Biophys. Acta 801:259-64). In addition a role of the enzyme in liver cancer has been proposed (Hoshino et al., Biochim. Biophys. Acta 719:518-526).

The ubiquitin protein ligase UBC9 from mouse (SEQ ID No. 69) or human (SEQ ID No. 70) (Yasugi and Howley; 1996, Nucleic Acids Res. 24:2005-2010; SwissProt: P50550), that is an important component of the proteasome mediated protein degradation (Hershko and Ciechanover, 1998, Annu. Rev. Biochem. 67:425-479). The ubiquitin dependent protein degradation plays a role in most divergent processes like cell cycle control, signal transduction or immune response. There are indications that UBC9 plays a role in accelerated aging (Kawabe et al., 2000, J. Biol. Chem.). In addition UBC9 catalyzes the sumoylation of p53 and thus activates its function as transcription factor (Rodriguez et al., 1999, EMBO J. 18:6455-61).

For none of these polypeptides, nucleic acids coding for them or the described cDNA a connection with skin disorders or wound healing or its disorders has been described or suggested. Therefore, it was unexpected that these compounds could be used according to the present invention. The accession numbers of the polypeptides according to the invention and the cDNAs are indicated in Table 4. The cDNA sequences of the polypeptides in SEQ ID No. 31, SEQ ID No. 35 and SEQ ID No. 37, SEQ ID No. 80 and SEQ ID No. 81, are indicated in SEQ ID No. 53 and SEQ ID No. 54 and SEQ ID No. 83 and SEQ ID No. 84.

During the analysis of gene expression during the wound healing processes it was possible to identify additional genes whose already known and described functions until now were not connected with wound healing, but whose regulation is essential for the wound healing process and which are thus brought for the first time in a causal relationship with wound healing. The polypeptides of these genes do not belong to the targets known until now for therapy in connection with pathological change of wound healing, such that completely novel therapeutic approaches result from this invention.

The object of the invention is therefore additionally achieved by the use of at least one polypeptide selected from a sequence of SEQ ID No. 21 to SEQ ID No. 26 and/or SEQ ID No. 29 to SEQ ID No. 30 and/or SEQ ID No. 71 to SEQ ID No. 73 or functional variants thereof and/or nucleic acids or variants thereof encoding these, and/or of a cell expressing said polypeptide or a functional variant thereof or said nucleic acid or variants thereof, if appropriate combined or together with suitable additives and/or auxiliaries, for the diagnosis, prevention and/or treatment in wound healing and/or its pathological disorders, or for the identification of pharmacologically active substances.

The following polypeptides can be used according to the invention.

The monocyte chemotactic protein-3, MCP-3 from mouse (SEQ ID No.21) or human (SEQ ID No. 22) (Kulmburg et al., 1992, J. Exp. Med. 176:1773-1778; Minty et al., 1993, Eur. Cytokine Netw. 4:99-110), that is known from WO 95/04158, WO 99/12968 and EP 0 488 900. MCP-3 is a CC-chemokine that serves the chemoattraction and activation of monocytes, T-lymphocytes, eosinophiles and basophilic granulocytes, natural killer cells and dendritic cells. The activation of the target cells is effected through the chemokine receptors CCR2 and CCR3 (Wang et al., 2000 Biochim. Biophys. Acta 1500:41-8). MCP-3 plays a role in allergic reactions of the skin (Ying et al., 1999, J. Immunol. 163:3976-84.

The alpha-chain of the heterodimeric Interleukin-5 receptor of mouse (SEQ ID No. 23) or human (SEQ ID No. 24) (Takaki 1990, EMBO J. 9:4367-4374; Tavernier et al., 1992, Proc. Natl. Acad. Sci. U.S.A. 89:7041-7045), that is known from EP 0 475 746 and WO 98/47923. It mediates the specific binding of the ligand Interleukin-5 (Van Ostade et al., 1999, Eur. J. Biochem. 259:954-60) and is expressed on the cell membrane of eosinophiles (Weltman & Karim, 1998, Allergy Asthma Proc. 19:257-61). A role of the Interleukin-5 receptors has been described for Atopic Dermatitis (Taha et al., 1998, J. Allergy Clin. Immunol. 102:245-50) Interleukin-5 plays an essential role in the differentiation, proliferation and functional activation of eosinophiles (Iwama et al., 1999, Mol. Cel. Biol. 19:3940-50) and in contrast to the receptor, that is described here, a function in wound healing has been described for Interleukin-5 (Yang et al., 1997, Am. J. Pathol. 151:813-9).

The integral membrane protein Dad1 from mouse (SEQ ID No. 25) or human (SEQ ID No. 26) (Nakashima et al., 1993, Mol. Cell. Biol. 13:6367-6374; Apte et al., 1995, FEBS Lett. 363:304-306). It is a component of the oligosaccharyl-transferase enzyme complexes that initiates the N-glycosylation (Sanjay et al., 1998, J. Biol. Chem. 273:26094-9). Dad1 plays a role in inhibition of apoptosis in particular cell types (Hong et al., 1999, J. Immunol. 163:1888-93) and in keloids (Sayah et al., 1999, J. Surg. Res. 87:209-16).

MCP-2 (C-C chemokine monocyte chemotactic protein 2) from human (SEQ ID No. 30) or mouse (SEQ ID No. 29) (van Coillie et al., 1997, Genomics 40:323-331; EMBL: AB023418) that is known from EP 0 905 240, EP 0 905 241, WO 98/02459, EP 0 906 954, WO 95/04158, WO 99/12968 and WO 97/25427. MCP-2 belongs to the C-C chemokines and acts as a chemoatractant for different cells like macrophages, basophiles and cosinophiles (Taub et al., 1995, J. Clin. Invest. 95:1370-6; Proost et al., 1996, J. Leukoc. Biol. 59:67-74). MCP-2 is a signal molecule that stimulates the directed migration of T-cells and monocytes in processes of inflammation and recruits them (Taub et al., 1995, J. Clin. Invest. 95:1370-6). In addition to the known polypeptide variants of human (SEQ ID No. 31) (Van Coillie et al., 1997 Genomics 40:323-331) the closely related polypeptide with a divergent sequence (SEQ ID No. 71), that is described in this work for the first time, can also be used.

The cysteine protease cathepsin C from mouse (SEQ ID No. 72) or human (SEQ ID No. 73) (Paris et al., 1995, FEBS Lett. 369:326-330; McGuire et al., 1997, Biochim. Biophys. Acta 1351:267-273), that is known from WO 96/33278, which is present in the lysosomes of different cells (Turk et al., 1997, Biol. Chem. 378:141-150). Cathepsin C plays an important role in the activation of granzym A and B and, thus, in induction of apoptosis through cytotoxic lymphocytes (Pham and Ley, 1999, Proc. Natl. Acad. Sci. U.S.A. 96:8627-8632). In addition it was observed that a “loss-of-function” mutation in the cathepsin C gene leads to palmoplanar keratosis and periodontitis (Hart et al., 1999, J. Med. Genet. 36:881-887; Toomes et al., 1999, Nat. Genet. 23:421-424).

For none of the polypeptides or nucleic acids coding for them a connection with wound healing was described or suggested until now. It was, therefore, unexpected that these polypeptides can be used according to the present invention. The accession numbers of the polypeptides according to the present invention and their cDNAs are indicated in Table 5.

Generally, the analysis of differentially expressed genes in tissues is affected by markedly more errors in the form of false-positive clones than the analysis of cell culture systems. This problem cannot be circumvented by the use of a defined cell culture system, as existing, simple cell culture systems cannot adequately simulate the complexity of the wound-healing process in the tissue.

The problem exists in particular in the skin, which consists of a multiplicity of different cell types. Moreover, the process of wound healing is a highly complicated process which includes temporal and spatial changes of cellular processes, such as proliferation and differentiation, in the different cell types. The approach to investigate not only the complex cell system skin, but moreover the physiological process of wound healing and even different wound-healing stages at the level of differentially expressed genes is therefore not a promising strategy for a person skilled in the art. On account of these difficulties, the success of the screening was significantly dependent on the choice of the experimental parameters. While the methods used (e.g. subtractive hybridization) are standard methods, the screening and verification strategy is already inventive per se owing to the thought-out and defined choice of parameters. For example, the time of biopsy taking is critical for the success of the screening: wound-healing disorders and skin diseases are often based on disorders in cell proliferation and cell migration. These processes are initiated one day after wounding, which is why analysis of the molecular processes before this time would yield little information about the processes which are essential for normally proceeding wound healing. On the other hand, in the course of wound healing, the composition of the cell types in the wound changes greatly later than one day after wounding. This can lead to a differential expression of a specific gene in the wound being measured which is based not on altered expression in the cells, but only on the different cell composition. This illustrates that the choice of the day of biopsy taking crucially affected the success of the screening. Despite the defined parameters, an overrepresentation of genes was observed, which are differentially expressed during wound healing, but which are unsuitable for use in wound healing or in skin diseases. These genes include, for example, genes which code for enzymes of the primary metabolism, such as glycolysis, citrate cycle, gluconeogenesis and respiratory chain, but also genes which code for ribosomal proteins, e.g. L41 and S20. Only a comparatively small number of genes were identified as suitable. An identification of the genes useable according to the invention as genes relevant to wound healing was therefore surprising.

Moreover, there are enormous variabilities in the state of the wound at the time of a possible biopsy of the patient on initial contact with the physician. An animal model was therefore used for the identification of the previously described nucleic acids. BALB/c mice were wounded and wound biopsies were taken at different times. This procedure has the advantage that conditions such as genetic background, nature of the wound, time of the biopsy etc. can be controlled exactly and therefore allow a reproducible analysis of gene expression. Even under the defined mouse conditions, further methodical problems arise such as redundancy of the analyzed clones and underrepresentation of weakly expressed genes, which make the identification of relevant genes difficult.

The nucleic acids of the polypeptides useable according to the invention were isolated from cDNA libraries which prepared from intact and wounded skin. The cDNAs selected here were those which have different frequency rates in well-healing wounds in comparison to poorly healing wounds (examples 1 and 3). This was carried out, for example, with the aid of subtractive hybridization (Diatchenko et al., 1996, Proc. Natl. Acad. Sci. USA 93: 6025-30) and/or using the comparative counting of clones in cDNA libraries by means of analysis of restriction fragment patterns (Halle et al., 1999, EP 0965642A1) and/or with the aid of “differential display RT-PCR” (Liang et al., 1992, Cancer Res. 52: 6996-6998; Liang and Pardee, 1992, Science 257: 967-971; Prashar and Weissman, 1996, Proc. Natl. Acad. Sci. USA 93: 659-663). The cDNAs thus selected originate from genes which are either more strongly or more weakly expressed in wound healing disorders than in wound healing which proceeds normally.

After the primary identification of a gene, it is necessary to confirm wound healing-specific expression by a further method. This was carried out with the aid of “reverse Northern blots” and “TaqMan analysis”. Using these methods, the amount of mRNA in tissues from various wound-healing states and in skin diseases (psoriasis) was determined or skin disease-specific local alterations in the expression pattern were detected in biopsies (Examples 2, 4 to 7).

In the present analysis of gene expression during the wound-healing process, besides genes whose function was completely unknown until now, genes were also identified which had previously not been linked to wound-healing disorders. Novel variants of known genes were furthermore identified having sequences which differed significantly from the previously published and/or patented sequences.

Of the part of the identified genes previously not connected with wound-healing disorders, it was hitherto known that they have a function in proliferation (Tsg101: Xie et al., Proc. Natl. Acad. Sci. USA 95: 1595-1600; MASPIN: Sager et al., 1997, Adv. Exp. Med. Biol. 425:77-88; B-Raf: Mason et al., 1999, EMBO J. 18:2137-48; Ikawa et al., 1988, Mol. Cell Biol. 8:2651-2654; Prothymosin alpha: Tao et al., 1999, J. Cell Physiol. 178:154-163; Eps8: Wong et al., 1994, Oncogene 9:3057-3061; KIAA1247: WO 99/34004; EAT/MCL-1: Tang et al., 1998, Clin. Cancer Res. 4:1865-1871; TSC-22: Kester et al., 1999, J. Biol. Chem. 274:27439-47; Fer: Morris et al., 1990, Cytogenet. Cell. Genet. 53:196-200), differentiation (MASPIN: Zhang et al., 1999, Dev. Biol. 215:278-87; Split hand/foot deleted 1: Crackower et al., 1996, Hum. Mol. Genet. 5:571-9), cell migration (MRP-3: Haelens et al., 1996, Immunobiology 195:499-521; MCP-3: Taub et al., 1995, J. Clin. Invest. 95:1370-6; MCP-2: Taub et al., 1995, J. Clin. Invest. 95:1370-6) and/or apoptosis (B-Raf: Erhardt et al., 1999, Mol. Cell. Biol. 19:530815). These genes, however, were previously not linked to wound healing.

In addition to the known polypeptides of human phospholipase inhibitor GIPL (U.S. Pat. No. 5,948,626), MCP-2 (van Coillie et al., 1997, Genomics 40: 323-331), BAF57 (WO 95/14772) and mouse cystatin C (Solem et al., 1990, Biochem. Biophys. Res. Commun. 172:945-951), closely related polypeptides having a significantly different sequence were identified. Of the known polypeptides of human phospholipase GIPL (U.S. Pat. No. 5,948,626) and human KIAA1247 (WO 99/34004), the sequences of the corresponding mouse polypeptide were identified for the first time.

The polypeptides of these genes do not include the previously known targets of therapies of wound-healing disorders, so that completely new therapeutic approaches result from this invention. Of the remaining identified genes, no description of function yet exists (Table 3).

For the checking or generation of full-length cDNA sequences of the previously described nucleic acids, full-length clones were generated with the aid of colony hybridization (Sambrook et al., 1989, Molecular Cloning: A Laboratory Manual, Cold Spring Harbor, Cold Spring Harbor Laboratory Press, New York, chapter 8-10) and/or PCR-based methods (“RACE”, Frohman et al., 1988, Proc. Natl. Acad. Sci. USA 85: 8998-9002, Chenchik et al., 1996, in A Laboratory Guide to RNA: Isolation, Analysis, and Synthesis, Ed. Krieg, Wiley-Liss, pages 272-321; “LDPCR”, Barnes, 1994, Proc. Natl. Acad. Sci. USA 91: 2216-20) both for the mouse genes and also for the human genes and the sequence of these clones was determined.

The term “functional variants” of a polypeptide within the meaning of the present invention includes polypeptides which are regulated, for example, like the polypeptides used according to the invention during disease, in particular skin diseases, or in regenerative processes of the skin, but in particular in wound-healing disorders. Functional variants, for example, also include polypeptides which are encoded by a nucleic acid which is isolated from non-skin-specific tissue, e.g. embryonic tissue, but after expression in a cell involved in wound healing or skin disease have the designated functions.

Functional variants within the meaning of the present invention are also polypeptides which have a sequence homology, in particular a sequence identity, of about 70%, preferably about 80%, in particular about 90%, especially about 95%, with the polypeptide having the amino acid sequence according to one of SEQ ID No. 1 to SEQ ID No. 48 and SEQ ID No. 55 to SEQ ID No. 58 and SEQ ID No. 63 to SEQ ID No. 73 and SEQ ID No. 80 to SEQ ID No. 82. Examples of such functional variants are accordingly the polypeptides homologous to a polypeptide useable according to the invention, which originate from organisms other than the human or the mouse, preferably from non-human mammals such as, for example monkeys, pigs and rats. Other examples of functional variants are polypeptides which are encoded by different alleles of the gene, in different individuals or in different organs of an organism.

Sequence identity is understood as degree of identity (=% positives) of two sequences, that in the case of polypeptides can be determined by means of for example BlastP 2.0.1 and in the case of nucleic acids by means of for example BLASTN 2.014, wherein the Filter is set off (Altschul et al., 1997, Nucleic Acids Res., 25:3389-3402).

“Functional Variants” of the polypeptide can also be parts of the polypeptide used according to the invention with at least 6 amino acids length, preferably with at least 8 amino acids length, in particular with at least 12 amino acids length. Also included are deletions of the polypeptides used accordingly to the invention, in the range from about 1-60, preferably from about 1-30, in particular from about 1-15, especially from about 1-5 amino acids. For example, the first amino acid methionine can be absent without the function of the polypeptide being significantly altered.

In order to decide, whether a candidate polypeptide is a functional variant, the activity of the candidate functional variant polypeptide may be compared with the activity of a polypeptide useable according to the invention in functional assays such as for example single cell or cell culture systems or standard wound healing assays. Assuming that the candidate functional variant polypeptide fulfills the criteria of a functional variant on the level of % sequence identity listed above the candidate functional variant molecule represents a functional variant if the activity in the functional assays is similar to or identical with the activity exhibited by the polypeptide useable according to the invention.

Such standard wound healing assays comprise for example the application of the an expression vector containing a nucleic acid coding for the candidate polypeptide or the application of the candidate polypeptide itself or of an antibody directed against the candidate polypeptide or of an antisense oligonucleotide to punched wounds, and after incubation for example of an expression vector comparing the progress of wound healing of wounds that have been injected with expression vectors containing e.g. the nucleic acid coding for the candidate functional variant polypeptide, with the progress of wound healing of wounds injected with an expression vector containing the nucleic acid coding for the polypeptide useable according to the invention, or containing a control vector with no insert. Such assays may also be applied test the activity of candidate functional variant polypeptides in the case of disorders of wound healing employing for example badly healing wounds of dexamethasone-treated animals. For example, it was demonstrated that application of the polypeptide-variants PDGF-A and PDGF-B on badly healing rabbit wounds resulted in a comparable wound healing response (J. Surg. Res., 2000, 93:230-236). Similar tests can be carried out for skin disorders, for example Psoriasis. In this case, an expression vector containing a nucleic acid coding for the candidate polypeptide or the candidate polypeptide itself or an antibody directed against the candidate polypeptide or an antisense oligonucleotide are applied to for example human afflicted skin areas transplanted onto SCID mice and the course of the skin disorder, for example the healing, is determined, for example by measuring PASI-score in the case of psoriasis.

The term “coding nucleic acid” relates to a DNA sequence which codes for an isolatable bioactive polypeptide according to the invention or a precursor. The polypeptide can be encoded by a sequence of full length or any part of the coding sequence as long as the specific, for example enzymatic, activity is retained.

It is known that small alterations in the sequence of the nucleic acids described above can be present, for example, due to the degeneration of the genetic code, or that untranslated sequences can be attached to the 5′ and/or 3′ end of the nucleic acid without its activity being significantly altered. Also included are modifications that are carried out as described below. This invention, therefore, also comprises so-called “variants” of the nucleic acids described above

“Variants” are understood as meaning all DNA sequences which are complementary to a DNA sequence, which hybridize with the reference sequence under stringent conditions and have a similar activity to the corresponding polypeptide according to the invention.

“Stringent hybridization conditions” are understood as meaning for example those conditions in which hybridization takes place at 60° C. in 2.5×SSC buffer, followed by a number of washing steps at 37° C. in a lower buffer concentration, and remains stable.

Variants of the nucleic acids can also be parts of the nucleic acids used according to the present invention with at least 8 nucleotides length, preferably with at least 18 nucleotides length, in particular with at least 24 nucleotides length particularly preferred with at least 30 nucleotides, and especially preferred with at least 42 nucleotides.

The term “pharmacologically active substance” in the sense of the present invention is understood as meaning all those molecules, compounds and/or compositions and substance mixtures which can interact under suitable conditions with the nucleic acids, polypeptides or antibodies or antibody fragments described above, if appropriate together with suitable additives and/or auxiliaries. Possible pharmacologically active substances are simple chemical organic or inorganic molecules or compounds, but can also include peptides, proteins or complexes thereof. Examples of pharmacologically active substances are organic molecules that are derived from libraries of compounds that have been analyzed for their pharmacological activity. On account of their interaction, the pharmacologically active substances can influence the function(s) of the nucleic acids, polypeptides or antibodies in vivo or in vitro or alternatively only bind to the nucleic acids, polypeptides or antibodies or antibody fragments described above or enter into other interactions of covalent or non-covalent manner with them.

The term “regulation” is understood, for example, as meaning the raising or lowering of the amount of polypeptide or nucleic acid encoding this. This may occur, for example, on the transcriptional or translational level.

The polypeptides according to the invention can furthermore be characterized in that they are synthetically prepared. Thus, the entire polypeptide or parts thereof can be synthesized, for example, with the aid of the conventional synthesis (Merrifield technique). Parts of the polypeptides according to the invention are particularly suitable to obtain antisera, with whose aid suitable gene expression banks can be searched in order thus to arrive at further functional variants of the polypeptide according to the invention.

Preferentially, the nucleic acids used according to the invention are DNA or RNA, preferably a DNA, in particular a double-stranded DNA. The sequence of the nucleic acids can furthermore be characterized in that it has at least one intron and/or one polyA sequence. The nucleic acids according to the invention can also be used in the form of their antisense sequence.

For the expression of the gene concerned, in general a double-stranded DNA is preferred, the DNA region coding for the polypeptide being particularly preferred. This region begins with the first start codon (ATG) lying in a Kozak sequence (Kozak, 1987, Nucleic. Acids Res. 15: 8125-48) up to the next stop codon (TAG, TGA or TAA), which lies in the same reading frame to the ATG.

A further use of the nucleic acid sequences according to the invention is the construction of anti-sense oligonucleotides (Zheng and Kemeny, 1995, Clin. Exp. Immunol. 100: 380-2; Nellen and Lichtenstein, 1993, Trends Biochem. Sci. 18: 419-23; Stein, 1992, Leukemia 6: 967-74) and/or ribozymes (Amarzguioui, et al. 1998, Cell. Mol. Life Sci. 54: 1175-202; Vaish, et al., 1998, Nucleic Acids Res. 26: 5237-42; Persidis, 1997, Nat. Biotechnol. 15: 921-2; Couture and Stinchcomb, 1996, Trends Genet. 12: 510-5). Using anti-sense oligonucleotides, the stability of the nucleic acid used according to the invention can be decreased and/or the translation of the nucleic acid used according to the invention inhibited. Thus, for example, the expression of the corresponding genes in cells can be decreased both in vivo and in vitro. Oligonuclecotides can therefore be suitable as therapeutics. This strategy is suitable, for example, for skin, epidermal and dermal cells, in particular if the antisense oligonucleotides are complexed with liposomes (Smyth et al., 1997, J. Invest. Dermatol. 108: 523-6; White et al., 1999, J. Invest. Dermatol. 112: 699-705; White et al., 1999, J. Invest. Dermatol. 112: 887-92). For use as a sample or as an “antisense” oligonucleotide, a single-stranded DNA or RNA is preferred.

Furthermore, a nucleic acid which has been prepared synthetically can be used for carrying out the invention. Thus, the nucleic acid according to the invention can be synthesized, for example, chemically with the aid of the DNA sequences described in Tables 3 to 5 and/or with the aid of the protein sequences likewise described in these tables with reference to the genetic code, e.g. according to the phosphotriester method (see, for example, Uhlmann, E. & Peyman, A. (1990) Chemical Reviews, 90, 543-584, No. 4).

As a rule, oligonucleotides are rapidly degraded by endo- or exonucleases, in particular by DNases and RNases occurring in the cell. It is therefore advantageous to modify the nucleic acid in order to stabilize it against degradation, so that a high concentration of the nucleic acid is maintained in the cell over a long period (Beigelman et al., 1995, Nucleic Acids Res. 23: 3989-94; Dudycz, 1995, WO 95/11910; Macadam et al., 1998, WO 98/37240; Reese et al., 1997, WO 97/29116). Typically, such stabilization can be obtained by the introduction of one or more internucleotide phosphorus groups or by the introduction of one or more non-phosphorus internucleotides.

Suitable modified internucleotides are summarized in Uhlmann and Peymann (1990 Chem. Rev. 90, 544) (see also Beigelman et al., 1995 Nucleic Acids Res. 23: 3989-94; Dudycz, 1995, WO 95/11910; Madadam et al., 1998, WO 98/37240; Reese et al., 1997, WO 97/29116). Modified internucleotide phosphate radicals and/or non-phosphorus bridges in a nucleic acid which can be employed in one of the uses according to the invention contain, for example, methylphosphonate, phosphorothioate, phosphoramidate, phosphorodithioate, phosphate ester, while non-phosphorus internucleotide analogues, for example, contain siloxane bridges, carbonate bridges, carboxymethyl esters, acetamidate bridges and/or thioether bridges. It is also intended that this modification should improve the stability of a pharmaceutical composition which can be employed in one of the uses according to the invention.

In a further embodiment of the use according to the invention, the nucleic acids are comprised in a vector, preferably in a “shuttle” vector, phagemid, cosmid, expression vector or vector applicable in gene therapy. Furthermore, the above mentioned nucleic acids can be included in “knock-out” gene constructs or expression cassettes.

Preferably, the vector applicable in gene therapy contains wound- or skin-specific regulatory sequences which are functionally associated with the nucleic acid according to the invention.

The expression vectors can be prokaryotic or eukaryotic expression vectors. Examples of prokaryotic expression vectors are, for expression in E. coli, e.g. the vectors pGEM or pUC derivatives, examples of eukaryotic expression vectors are for expression in Saccharomyces cerevisiae, e.g. the vectors p426Met25 or p426GAL1 (Mumberg et al. (1994) Nucl. Acids Res., 22, 5767-5768), for expression in insect cells, e.g. Baculovirus vectors such as disclosed in EP-B1-0 127 839 or EP-B1-0 549 721, and for expression in mammalian cells, e.g. the vectors Rc/CMV and Rc/RSV or SV40 vectors, which are all generally obtainable.

In general, the expression vectors also contain promoters suitable for the respective host cell, such as, for example, the trp promoter for expression in E. coli (see, for example, EP-B1-0 154 133), the MET 25, GAL 1 or ADH2 promoter for expression in yeasts (Russel et al. (1983), J. Biol. Chem. 258, 2674-2682; Mumberg, supra), the Baculovirus polyhedrin promoter, for expression in insect cells (see, for example, EP-B1-0 127 839). For expression in mammalian cells, for example, suitable promoters are those which allow a constitutive, regulatable, tissue-specific, cell-cycle-specific or metabolically specific expression in eukaryotic cells. Regulatable elements according to the present invention are promoters, activator sequences, enhancers, silencers and/or repressor sequences.

Examples of suitable regulatable elements which make possible constitutive expression in eukaryotes are promoters which are recognized by the RNA polymerase III or viral promoters, CMV enhancer, CMV promoter, SV40 promoter or LTR promoters, e.g. from MMTV (mouse mammary tumor virus; Lee et al. (1981) Nature 214, 228-232) and further viral promoter and activator sequences, derived from, for example, HBV, HCV, HSV, HPV, EBV, HTLV or HIV.

Examples of regulatable elements which make possible regulatable expression in eukaryotes are the tetracycline operator in combination with a corresponding repressor (Gossen M. et al. (1994) Curr. Opin. Biotechnol. 5, 516-20).

Preferably, the expression of wound-healing-relevant genes takes place under the control of tissue-specific promoters, wherein skin-specific promoters such as, for example, the human K10 promoter (Bailleul et al., 1990. Cell 62: 697-708), the human K14 promoter (Vassar et al., 1989, Proc. Natl. Acad. Sci. USA 86: 1563-67), the bovine cytokeratin IV promoter (Fuchs et al., 1988; The biology of wool and hair (ed. G. E. Rogers, et al.), pp. 287-309. Chapman and Hall, London/New York) are particularly to be preferred.

Further examples of regulatable elements which make possible tissue-specific expression in eukaryotes are promoters or activator sequences from promoters or enhancers of those genes which code for proteins which are only expressed in certain cell types.

Examples of regulatable elements which make possible cell cycle-specific expression in eukaryotes are promoters of the following genes: cdc25A, cdc25B, cdc25C, cyclin A, cyclin E, cdc2, E2F-1 to E2F-5, B-myb or DHFR (Zwicker J. and Müller R. (1997) Trends Genet. 13, 3-6). The use of cell cycle regulated promoters is particularly preferred in cases, in which expression of the polypeptides or nucleic acids used according to the invention is to be restricted to proliferating cells.

An example of a regulatable element which makes possible the keratinocyte-specific expression in the skin, is the FiRE-element (Jaakkola et al., 2000, Gen. Ther., 7: 1640-1647). The FiRE element is an AP-1-driven, FGF-inducible response element of the Syndecan-1 gene (Jaakkola et al., 1998, FASEB J., 12: 959-9).

Examples of regulatable elements which make possible metabolically specific expression in eukaryotes are promoters which are regulated by hypoxia, by glucose deficiency, by phosphate concentration or by heat shock.

In order to make possible the introduction of nucleic acids as described above and thus the expression of the polypeptide in a eu- or prokaryotic cell by transfection, transformation or infection, the nucleic acid can be present as a plasmid, as part of a viral or non-viral vector. Suitable viral vectors here are particularly: baculoviruses, vaccinia viruses, adenoviruses, adeno-associated viruses and herpesviruses. Suitable non-viral vectors here are particularly: virosomes, liposomes, cationic lipids, or poly-lysine-conjugated DNA.

Examples of vectors having gene therapy activity are virus vectors, for example adenovirus vectors or retroviral vectors (Lindemann et al., 1997, Mol. Med. 3: 466-76; Springer et al., 1998, Mol. Cell. 2: 549-58). Eukaryotic expression vectors are suitable in isolated form for gene therapy use, as naked DNA can penetrate into skin cells on topical application (Hengge et al., 1996, J. Clin. Invest. 97: 2911-6; Yu et al., 1999, J. Invest. Dermatol. 112: 370-5).

Vectors having gene therapy activity can also be obtained by complexing the nucleic acid with liposomes, since a very high transfection efficiency, in particular of skin cells, can thus be achieved (Alexander and Akhurst, 1995, Hum. Mol. Genet. 4: 2279-85). In the case of lipofection, small unilamellar vesicles are prepared from cationic lipids by ultrasonic treatment of the liposome suspension. The DNA is bound ionically to the surface of the liposomes, namely in such a ratio that a positive net charge remains and the plasmid DNA is complexed to 100% of the liposomes. In addition to the lipid mixtures DOTMA (1, 2-dioleyloxypropyl-3-trimethylammonium bromide) and DPOE (dioleoylphosphatidylethanolamine) employed by Felgner et al. (1987, supra), meanwhile numerous novel lipid formulations were synthesized and tested for their efficiency in the transfection of various cell lines (Behr et al. 1989, Proc. Natl. Acad. Sci. USA 86: 6982-6986; Felgner et al., 1994, J. Biol. Chem. 269:2550-2561; Gao, X. and Huang, 1991, Biochim. Biophys. Acta 1189:195-203). Examples of the novel lipid formulations are DOTAP N-[1-(2,3-dioleoyloxy)propyl]-N,N,N-trimethylammonium ethyl-sulphate or DOGS (TRANSFECTAM; dioctadecylamidoglycylspermine). Other lipids suitable for transfection in keratinocytes in vivo and in vitro are the cationic lipids Cytofectin GS 2888 (U.S. Pat. No. 5,777,153; Lewis et al., 1996, Proc. Natl. Acad. Sci. USA, 93: 3176-3181). Auxiliaries which increase the transfer of nucleic acids into the cell can be, for example, proteins or peptides which are bound to DNA or synthetic peptide-DNA molecules which make possible the transport of the nucleic acid into the nucleus of the cell (Schwartz et al., 1999, Gene Therapy 6:282; Branden et al., 1999, Nature Biotech. 17:784). Auxiliaries also include molecules which make possible the release of nucleic acids into the cytoplasm of the cell (Planck et al., 1994, J. Biol. Chem. 269:12918; Kichler et al. (1997) Bioconj. Chem. 8:213) or, for example, liposomes (Uhlmann and Peymann, 1990, supra). Another particularly suitable form of gene therapy vectors can be obtained by applying the above described nucleic acid to gold particles and shooting these into tissue, preferably into the skin, or cells with the aid of the so-called gene gun (Wang et al., 1999, J. Invest. Dermatol. 112: 775-81, Tuting et al., 1998, J. Invest. Dermatol. 111: 183-8).

A further form of a vector applicable in gene therapy can be prepared by the introduction of “naked” expression vectors into a biocompatible matrix, for example a collagen matrix. This matrix can be introduced into wounds in order to transfect the immigrating cells with the expression vector and to express the polypeptides according to the invention in the cells (Goldstein and Banadio, U.S. Pat. No. 5,962,427).

For gene therapy use of the above described nucleic acid, it is also advantageous if the part of the nucleic acid which codes for the polypeptide contains one or more non-coding sequences including intron sequences, preferably between promoter and the start codon of the polypeptide, and/or a polyA sequence, in particular the naturally occurring polyA sequence or an SV40 virus polyA sequence, especially at the 3′ end of the gene, as a stabilization of the mRNA can be achieved thereby (Palmiter et al., 1991, Proc. Natl. Acad. Sci. USA 88:478-482; Jackson, 1993, Cell 74:9-14).

Knock-out gene constructs are known to the person skilled in the art, for example, from the U.S. Pat. Nos. 5,625,122; 5,698,765; 5,583,278 and 5,750,825.

A further preferred embodiment of the present invention is the use of a cell, preferentially of an autologous or heterologous cell, in particular a skin cell, which is transformed with a vector useable according to the invention or with a knock-out gene construct, for the diagnosis and/or prevention and/or treatment of diseases of skin cells and/or of wound healing and/or of their pathological disorders, and/or for the identification of pharmacologically active substances. Cells can be either prokaryotic or eukaryotic cells; examples of prokaryotic cells are E. coli and of eukaryotic cells are Saccharomyces cerevisiae or insect cells.

A particularly preferred transformed host cell is a transgenic embryonic non-human stem cell, which is characterized in that it comprises at least one knock-out gene construct and/or an expression cassette as described above. Processes for the transformation of host cells and/or stem cells are well known to the person skilled in the art and include, for example, electroporation or microinjection.

The genome of transgenic non-human mammals comprises at least one knock-out gene construct and/or an expression cassette as described above. Transgenic animals in general show a tissue-specifically increased expression of the nucleic acids and/or polypeptides and can be used for the analysis of wound healing disorders. Thus, for example, an activin A transgenic mouse exhibits improved wound healing (Munz et al., 1999, EMBO J. 18: 5205-15) while a transgenic mouse having a dominantly negative KGF receptor exhibits delayed wound healing (Werner et al., 1994, Science 266: 819-22).

Processes for the preparation of transgenic animals, in particular of transgenic mice, are likewise known to the person skilled in the art from DE 196 25 049 and U.S. Pat. No. 4,736,866; U.S. Pat. No. 5,625,122; U.S. Pat. No. 5,698,765; U.S. Pat. No. 5,583,278 and U.S. Pat. No. 5,750,825 and include transgenic animals which can be produced, for example, by means of direct injection of expression vectors (see above) into embryos or spermatocytes or by means of the transfection of expression vectors into embryonic stem cells (Polites and Pinkert: DNA Microinjection and Transgenic Animal Production, page 15 to 68 in Pinkert, 1994: Transgenic animal technology: a laboratory handbook, Academic Press, London, UK; Houdebine, 1997, Harwood Academic Publishers, Amsterdam, The Netherlands; Doetschman: Gene Transfer in Embryonic Stem Cells, page 115 to 146 in Pinkert, 1994, supra; Wood: Retrovirus-Mediated Gene Transfer, page 147 to 176 in Pinkert, 1994, supra; Monastersky: Gene Transfer Technology; Alternative Techniques and Applications, page 177 to 220 in Pinkert, 1994, supra).

If the above described nucleic acids are integrated into so-called “targeting” vectors or “knock-out” gene constructs (Pinkert, 1994, supra), it is possible after transfection of embryonic stem cells and homologous recombination, for example, to generate knock-out mice which, in general, as heterozygous mice, show decreased expression of the nucleic acid, while homozygous mice no longer exhibit expression of the nucleic acid. The animals thus produced can also be used for the analysis of wound healing disorders. Thus, for example, the eNOS (Lee et al., 1999, Am. J. Physiol. 277: H1600-1608), Nf-1 (Atit et al., 1999, J. Invest. Dermatol. 112: 835-42) and osteopontin (Liaw et al., 1998, J. Clin. Invest. 101: 967-71) knock-out mice exhibit impaired wound healing. Here too, a tissue-specific reduction of the expression of wound healing-relevant genes, for example in skin-specific cells using the Cre-loxP system (stat3 knock-out, Sano et al., EMBO J 1999 18: 4657-68), is particularly to be preferred. Transgenic and knockout cells or animals produced in this way can also be used for the screening and for the identification of pharmacologically active substances vectors having gene therapy activity.

Polypeptides useable according to the invention can be prepared according to generally known recombinant processes. Furthermore, polypeptides useable according to the invention can be isolated from an organism or from tissue or cells and used according to the invention. Thus, it is possible, for example, to purify polypeptides useable according to the invention from mammal tissue, for example from skin or body fluids such as for example blood, serum, saliva, synovial fluid, wound liquid. Furthermore, starting from cells expressing polypeptides useable according to the invention, cell lines can be prepared which can then be used for the isolation of polypeptides useable according to the invention. For example skin cells, such as for example HaCaT cells can be transformed with expression vectors containing nucleic acids useable according to the invention. The expression can be for example constitutive or inducible.

The polypeptide is prepared, for example, by expression of the above described nucleic acids in a suitable expression system, as already mentioned above, according to the methods generally known to the person skilled in the art. Suitable host cells are, for example, the E.coli strains DHS, HB101 or BL21, the yeast strain Saccharomyces cerevisiae, the insect cell line Lepidoptera, e.g. from Spodoptera frugiperda, or the animal cells COS, Vero, 293, HaCaT, and HeLa, which are all generally obtainable.

A further embodiment relates to the use of the polypeptides according to the invention, the polypeptides being employed in the form of a fusion protein. Fusion proteins useable according to the invention can be prepared, for example, by expressing nucleic acids useable according to the invention of a suitable cell.

The fusion proteins useable according to the invention themselves already having the function of a polypeptide of the invention or the specific function being functionally active only after cleavage of the fusion portion. Especially included here are fusion proteins having a proportion of about 1-300, preferably about 1-200, in particular about 1-100, especially about 1-50, foreign amino acids. Examples of such peptide sequences are prokaryotic peptide sequences, which can be derived, for example, from the galactosidase of E.coli. Furthermore, viral peptide sequences, such as, for example, of the bacteriophage M13 can also be used in order thus to produce fusion proteins for the phage display process known to the person skilled in the art.

Further preferred examples of peptide sequences for fusion proteins are peptides, that facilitate easier detection of the fusion proteins, these are, for example, “Green-fluorescent-protein” or functional variants thereof (WO 95/07463).

For the purification of the proteins described above (a) further polypeptide(s) (tag) can be attached. Protein tags according to the invention allow, for example, high-affinity absorption to a matrix, stringent washing with suitable buffers without eluting the complex to a noticeable extent and subsequently targeted elution of the absorbed complex. Examples of the protein tags known to the person skilled in the art are a (His)₆ tag, a Myc tag, a FLAG tag, a haemagglutinin tag, glutathione transferase (GST) tag, intein having an affinity chitin-binding tag or maltose-binding protein (MBP) tag. These protein tags can be situated N- or C-terminally and/or internally.

A further embodiment of the invention relates to the use of an antibody or an antibody fragment directed against a polypeptide useable according to the invention or a functional variant thereof, preferably of a polyclonal or monoclonal antibody or antibody fragment, for the analysis, diagnosis, prevention and/or treatment of diseases of skin cells, of wound healing and/or disorders of wound healing, and its use for the identification of pharmacologically active substances, if appropriate combined or together with suitable additives and/or auxiliaries.

Thus the local injection, for example, of monoclonal antibodies against TGF beta 1 in the animal model can improve wound healing (Ernst et al., 1996, Gut 39:172-5).

The process for manufacturing an antibody or an antibody fragment is carried out according to methods generally known to the person skilled in the art by immunizing a mammal, for example a rabbit, with said polypeptide or parts thereof having at least 6 amino acid length, preferably having at least 8 amino acid length, in particular having at least 12 amino acid length, if appropriate in the presence of, for example, Freund's adjuvant and/or aluminium hydroxide gels (see, for example, Diamond et al., 1981, The New England Journal of Medicine, 1344-1349). The polyclonal antibodies formed in the animal as a result of an immunological reaction can then be easily isolated from the blood according to generally known methods and purified, for example, by means of column chromatography. Monoclonal antibodies can be produced, for example, according to the known method of Winter & Milstein (Winter, G. & Milstein, C. (1991) Nature, 349, 293-299). As alternatives to the classical antibodies, for example, “anti-calins” based on lipocalin can be used (Beste et al., 1999, Proc. Natl. Acad. Sci. USA, 96:1898-1903). The natural ligand-binding sites of the lipocalins, such as the retinol-binding protein or the bilin-binding protein can be modified, for example, by a “combinatorial protein design” approach in a manner such that they bind to selected haptens, for example to the polypeptides useable according to the invention (Skerra, 2000, Biochim. Biophys. Acta 1482:337-50). Further known “scaffolds” are known as alternatives for antibodies for molecular recognition (Skerra, J. Mol. Recognit., 2000, 13:167-187).

The antibody useable according to the invention or the antibody fragment is directed against a polypeptide according to the invention and reacts specifically with the polypeptides according to the invention, where the above mentioned parts of the polypeptide either are immunogenic themselves or can be rendered immunogenic or increased in their immunogenicity by coupling to suitable carriers, such as bovine serum albumin. This antibody useable according to the invention is either polyclonal or monoclonal; a monoclonal antibody is preferred. The term antibody or antibody fragment is understood according to the present invention as also meaning antibodies or antigen-binding parts thereof prepared by genetic engineering and optionally modified, such as chimeric antibodies, humanized antibodies, multifunctional antibodies, bi- or oligospecific antibodies, single-stranded antibodies, F(ab) or F(ab)₂ fragments (see, for example, EP-B1-0 368 684, U.S. Pat. Nos. 4,816,567, 4,816,397, WO 88/01649, WO 93/06213, WO 98/24884).

The identified pharmacologically active substances can be used, if appropriate combined or together with suitable additives and/or auxiliaries, for the production of a diagnostic or of a medicament for the prevention, treatment and/or diagnosis of diseases of skin cells and/or in wound healing and/or their pathological disorders.

In order to use nucleic acids as a diagnostic the polymerase chain reaction can be employed as described below. For the use of nucleic acids as a medicament, a vector applicable for gene therapy or antisense nucleotides can be utilized as described.

In order to use other organic or anorganic pharmacologically active substances as a medicament, they can be applied as described above. Antibodies can be utilized as a diagnostic by means of immunological techniques as described above, for example by using antibodies that are labeled with an enzyme. The specific antibody-peptide complex can be determined easily and quickly by means of an enzymatic color-reaction.

In order to use pharmacologically active substances as a diagnostic, the substances may contain a detectable marker, for example the substance may be radioactively labeled, fluorescence-labeled or luminescence-labeled. In addition substances may be coupled to enzymes that allow indirect detection, for example by enzymatic catalysis by means of peroxidase-assay using a chromogenic substrate as described above or by binding of a labeled or detectable antibody. The substances can be brought into contact with the sample and thus the amount of polypeptides useable according to the invention or a functional variant thereof or nucleic acids coding for this or a variant thereof, or a cell containing a polypeptide useable according to the invention or a functional variant thereof or a nucleic acid coding for this, or an antibody directed against a polypeptide useable according to the invention or a fragment thereof, can be determined. The result of the sample, being isolated from an organism to be analyzed, can be compared with the result of a sample, of a healthy or a pathological organism.

The present invention also relates to the use of at least one polypeptide useable according to the invention or a functional variant thereof and/or of a nucleic acid encoding this or a variant thereof, and/or of a cell expressing a polypeptide useable according to the invention or of a functional variant thereof or a nucleic acid encoding this or a variant thereof, and/or of an antibody or an antibody fragment directed against a polypeptide useable according to the invention, optionally combined or together with suitable additives and/or auxiliaries, for the production of a medicament for the prevention and/or treatment of diseases of skin cells, of wound healing and/or their pathological disorders.

The medicament useable according to the invention may be used for the prevention and/or treatment of diseases of skin cells, of wound healing and/or their pathological disorders, wherein at least one polypeptide useable according to the invention or a functional variant thereof or a nucleic acid encoding this, and/or a cell expressing a polypeptide useable according to the invention or a functional variant thereof or a nucleic acid encoding this or a variant thereof, and/or an antibody or an antibody fragment directed against a polypeptide useable according to the invention or a functional variant thereof, if appropriate combined or together with suitable additives and/or auxiliaries, is being employed.

The therapy of the disorders, of skin disorders, of wound healing and/or disorders of wound healing, can be carried out in a conventional manner, e.g. by means of dressings, plasters, compresses or gels which contain the medicaments according to the invention. It is thus possible to administer the pharmaceuticals containing the suitable additives and/or auxiliaries, such as, for example, physiological saline solution, demineralized water, stabilizers, proteinase inhibitors, gel formulations, such as, for example, white petroleum jelly, highly liquid paraffin and/or yellow wax, etc., topically and locally in order to influence wound healing immediately and directly. The administration of the medicaments according to the invention can furthermore also be carried out topically and locally in the area of the wound, if appropriate in the form of liposome complexes or gold particle complexes. Furthermore, the treatment can be carried out by means of a transdermal therapeutic system (TTS), which makes possible a temporally controlled release of the medicaments according to the invention. The treatment by means of the medicaments according to the invention, however, can also be carried out by means of oral dosage forms, such as, for example, tablets or capsules, by means of the mucous membranes, for example the nose or the oral cavity, or in the form of dispositories implanted under the skin. TTS are known for example, from EP 0 944 398 A1, EP 0 916 336 A1, EP 0 889 723 A1 or EP 0 852 493 A1.

For gene therapy use in humans, an especially suitable medicament is one which contains the described nucleic acid in naked form or in the form of one of the vectors having gene therapy activity described above or in a form complexed with liposomes or gold particles. The pharmaceutical carrier is, for example, a physiological buffer solution, preferably having a pH of about 6.0-8.0, preferably of about 6.8-7.8, in particular of about 7.4, and/or an osmolarity of about 200-400 milliosmol/liter, preferably of about 290-310 milliosmol/liter. In addition, the pharmaceutical carrier can contain suitable stabilizers, such as nuclease inhibitors, preferably complexing agents such as EDTA and/or other auxiliaries known to the person skilled in the art.

The nucleic acid described is optionally administered in the form of the virus vectors described above in greater detail or as liposome complexes or a gold particle complex, customarily topically and locally in the area of the wound. It is also possible to administer the polypeptide itself with suitable additives and/or auxiliaries, such as physiological saline solution, demineralized water, stabilizers, protease inhibitors, gel formulations, such as white petroleum jelly, highly liquid paraffin and/or yellow wax, etc., in order to affect wound healing immediately and directly.

Examples of disorders of skin cells within the meaning of the invention is understood as psoriasis, eczema, especially atopic eczema, acne, Urticaria, disorders of pigmentation of the skin, especially vitiligo, senile skin and disorders of hair growth and hair metabolism.

Wound healing within the meaning of the invention is understood as the healing process of a mechanical wound of the skin, such as for example laceration, skin abrasion or excoriation of the skin, for example by means of a permanent load, for example decubitus or necrotic processes, for example Necrobiosis lipoidica.

Examples of disorders of wound healing in the meaning of the invention comprise wounds of patients suffering from diabetes or alcoholism, wounds infected with organisms or viruses, ischemic wounds, wounds of patients suffering from arterial disorders, or venous insufficiency, and scars, preferably overshooting scars, especially keloids. Especially preferred badly healing wounds comprise diabetic, neuropathic, venous or arterial ulcers, especially diabetic ulcers.

The present invention furthermore relates to the use of at least one polypeptide useable according to the invention or a functional variant thereof and/or of a nucleic acid encoding this or a variant thereof, and/or of a cell expressing a polypeptide useable according to the invention or a functional variant thereof or a nucleic acid encoding this or a variant thereof, and/or of an antibody or of an antibody fragment directed against a polypeptide useable according to the invention or a functional variant thereof, if appropriate combined or together with suitable additives and/or auxiliaries, for the production of a diagnostic for the diagnosis of diseases of skin cells and/or in wound healing and/or their pathological disorders.

For example, it is possible according to the present invention to prepare a diagnostic based on the polymerase chain reaction (Examples 2, 4 to 7, PCR diagnostic, e.g. according to EP 0 200 362) or an RNase protection assay (see, for instance, Sambrook et al., supra chapter 7, page 7.71-7.78, Werner et al., 1992, Growth Factor and Receptors: A Practical Approach 175-197, Werner, 1998, Proc. Natl. Acad. Sci. U.S.A. 89: 6896-699) with the aid of a nucleic acid as described above. These tests are based on the specific hybridization of a nucleic acid with its complementary counter strand, usually of the corresponding mRNA or its cDNA. The nucleic acid described above can in this case also be modified, such as disclosed, for example, in EP 0 063 879. Preferably a DNA fragment is labeled according to generally known methods by means of suitable reagents, e.g. radioactively with α-P³²-dCTP or non-radioactively with biotin or digoxigenin, and incubated with isolated RNA, which has preferably been bound beforehand to suitable membranes of, for example, cellulose or nylon. With the same amount of investigated RNA from each tissue sample, the amount of mRNA which was specifically labeled by the sample can thus be determined. Alternatively, the determination of mRNA amount can also be carried directly out in tissue sections with the aid of in situ hybridization (Werner et al., 1992, Proc. Natl. Acad. Sci. USA 89: 6896-6900).

The diagnostic useable according to the invention is used for the diagnosis of diseases of skin cells and/or in wound healing and/or their pathological disorders, wherein at least one polypeptide useable according to the invention or a functional variant thereof and/or a nucleic acid encoding this or a variant thereof, and/or a cell expressing a polypeptide useable according to the invention or a functional variant thereof or nucleic acid coding for this or a variant thereof, and/or an antibody or an antibody fragment directed against a polypeptide useable according to the invention or a functional variant thereof, if appropriate combined or together with suitable additives and/or auxiliaries, is employed.

The diagnostic useable according to the invention, can thus also be used to specifically measure the strength of expression in a tissue sample in order to be able to safely diagnose, for example, a wound healing disorder or dermatological disorders (Examples 2, 4 to 7). Such a process is particularly suitable for the early prognosis of disorders.

A preferred diagnostic useable according to the invention contains the described polypeptide or the immunogenic parts thereof described in greater detail above. The polypeptide or the parts thereof, which are preferably bound to a solid phase, e.g. of nitrocellulose or nylon, can be brought into contact in vitro, for example, with the body fluid to be investigated, e.g. wound secretion, in order thus to be able to react, for example, with autoimmune antibodies. The antibody-peptide complex can then be detected, for example, with the aid of labeled anti-human IgG or antihuman IgM antibodies. The labeling involves, for example, an enzyme, such as peroxidase, which catalyses a color reaction. The presence and the amount of autoimmune antibody present can thus be detected easily and rapidly by means of the color reaction.

A further diagnostic useable according to the invention, that is that subject matter of the present invention, contains the antibodies useable according to the invention themselves. With the aid of these antibodies, it is possible, for example, to easily and rapidly investigate a tissue sample as to whether the concerned polypeptide is present in an increased amount in order to thereby obtain an indication of possible disorders, in particular skin disorder, and wound healing disorder. In this case, the antibodies according to the invention are labeled, for example, with an enzyme, as already described above. The specific antibody-peptide complex can thereby be detected easily and also rapidly by means of an enzymatic color reaction.

A further diagnostic useable according to the invention comprises a sample, preferably a DNA sample, and/or primer. This opens up a further possibility of obtaining the described nucleic acids, for example by isolation from a suitable gene bank, for example from a wound-specific gene bank, with the aid of a suitable sample (see, for example, J. Sambrook et al., 1989, Molecular Cloning. A Laboratory Manual 2nd edn., Cold Spring Harbor Laboratory, Cold Spring Harbor, N.Y. Chapter 8 page 8.1 to 8.81, Chapter 9 page 9.47 to 9.58 and Chapter 10 page 10.1 to 10.67).

Suitable samples are, for example, DNA or RNA fragments having a length of about 100-1000 nucleotides, preferably having a length of about 200-500 nucleotides, in particular having a length of about 300-400 nucleotides, whose sequence can be derived from the polypeptides according to SEQ ID No. 1 to SEQ ID No. 48, SEQ ID No. 55 to SEQ ID No. 58 and SEQ ID No. 63 to SEQ ID No. 73 and SEQ ID No. 80 to SEQ ID No. 82 of the sequence protocol and/or with the aid of the cDNA sequences of the database entries indicated in Tables 3 to 5 or with the aid of the sequence protocol according to SEQ ID No. 50 to SEQ ID No. 54 and SEQ ID No. 83 to SEQ ID No. 84.

Alternatively, it is possible with the aid of the derived nucleic acid sequences to synthesize oligonucleotides which are suitable as primers for a polymerase chain reaction. Using this, the nucleic acid described above or parts of this can be amplified and isolated from cDNA, for example wound-specific cDNA (Example 2). Suitable primers are, for example, DNA fragments having a length of about 10 to 100 nucleotides, preferably having a length of about 15 to 50 nucleotides, in particular having a length of 20 to 30 nucleotides, whose sequence can be derived from the polypeptides according to SEQ ID No. 1 to SEQ ID No. 48, SEQ ID No. 55 to SEQ ID No. 58 and SEQ ID No. 63 to SEQ ID No. 73 and SEQ ID No. 80 to SEQ ID No. 82 of the sequence protocol and/or with the aid of the cDNA sequences of the database entries indicated in Tables 3 to 5 or with the aid of the sequence protocol according to SEQ ID No. 50 to SEQ ID No. 54 and SEQ ID No. 83 to SEQ ID No. 84.

The present invention also relates to the use of at least one antibody or antibody fragment directed against a polypeptide useable according to the invention for the identification of pharmacologically active substances, wherein the antibody/antibodies or antibody fragment(s) is/are bound to a solid phase.

The present invention furthermore relates to the use of at least one polypeptide useable according to the invention or a functional variant thereof and/or of a nucleic acid encoding this or a variant thereof, and/or of a cell expressing a polypeptide useable according to the invention or a functional variant thereof or a nucleic acid coding for this or a variant thereof, and/or of an antibody or an antibody fragment directed against a polypeptide useable according to the invention or a functional variant thereof, if appropriate combined or together with suitable additives and/or auxiliaries, for the production of a test for finding pharmacologically active substances in connection with skin diseases and/or in connection with wound healing, in particular wound healing disorders.

At least one polypeptide useable according to the invention or a functional variant thereof and/or of a nucleic acid encoding this or a variant thereof, and/or of a cell expressing a polypeptide useable according to the invention or a functional variant thereof or a nucleic acid coding for this or a variant thereof, and/or of an antibody or an antibody fragment directed against a polypeptide useable according to the invention or a functional variant thereof, if appropriate combined or together with suitable additives and/or auxiliaries, may be used in the form of a test for finding pharmacologically active substances in connection with diseases of skin cells and/or in wound healing and/or their pathological disorders.

In a preferred embodiment of the invention the test system comprises at least one polypeptide useable according to the invention and/or at least one antibody or antibody fragment useable according to the invention, which is bound to a solid-phase.

In another preferred embodiment of the invention the test system comprises at least one cell expressing at least one polypeptide useable according to the invention or a nucleic acid coding for this.

A suitable system can be produced, for example, by the stable transformation of epidermal or dermal cells with expression vectors which contain selectable marker genes and the described nucleic acids. In this process, the expression of the described nucleic acids is altered in the cells such that it corresponds to the pathologically disturbed expression in vivo. Anti-sense oligonucleotides which contain the described nucleic acid can also be employed for this purpose. It is therefore of particular advantage for these systems to know the expression behavior of the genes in disturbed regenerative processes, such as disclosed in this application. Often, the pathological behavior of the cells in vitro can thus be mimicked and substances can be sought which reproduce the normal behavior of the cells and which have a therapeutic potential.

Suitable cells for these test systems useable according to the invention are, for example, HaCaT cells, which are generally obtainable, and the expression vector pCMV4 (Anderson et al., 1989, J. Biol. Chem. 264: 8222-9). The nucleic acid as described above can in this case be integrated into the expression vectors both in the sense and in the anti-sense orientation, such that the functional concentration of mRNA of the corresponding genes in the cells is either increased, or is decreased by hybridization with the antisense RNA. After the transformation and selection of stable transformants, the cells in culture in general show an altered proliferation, migration and/or differentiation behavior in comparison with control cells. This behavior in vitro is often correlated with the function of the corresponding genes in regenerative processes in the body (Yu et al., 1997, Arch. Dermatol. Res. 289: 352-9; Mils et al., 1997, Oncogene 14: 15555-61; Charvat et al., 1998, Exp Dermatol 7: 184-90; Werner, 1998, Cytokine Growth Factor Rev. 9: 153-65; Mythily et al., 1999, J. Gen. Virol. 80: 1707-13;) and can be detected using tests which are simple and rapid to carry out, such that test systems for pharmacologically active substances based thereon can be constructed. Thus, the proliferation behavior of cells can be detected very rapidly by, for example, the incorporation of labeled nucleotides into the DNA of the cells (see, for example, Savino and Dardenne, 1985, J. Immunol. Methods 85: 221-6; Perros and Weightman, 1991, Cell Prolif. 24: 517-23; Fries and Mitsuhashi, 1995, J. Clin. Lab. Anal. 9: 89-95), by staining the cells with specific stains (Schulz et al., 1994, J. Immunol. Methods 167: 1-13) or by means of immunological processes (Frahm et al., 1998, J. Immunol. Methods 211: 43-50). The migration can be detected simply by the migration index test (Charvat et al., supra) and comparable test systems (Benestad et al., 1987, Cell Tissue Kinet. 20: 109-19, Junger et al., 1993, J. Immunol. Methods 160: 73-9). Suitable differentiation markers are, for example, keratin 6, 10 and 14 and also loricrin and involucrin (Rosenthal et al., 1992, J. Invest. Dermatol. 98: 343-50), whose expression can be easily detected, for example, by means of generally obtainable antibodies.

Another suitable test system useable according to the invention is based on the identification of interactions using the so-called two-hybrid system (Fields and Sternglanz, 1994, Trends in Genetics, 10, 286-292; Colas and Brent, 1998 TIBTECH, 16, 355-363). In this test, cells are transformed using expression vectors which express fusion proteins from the polypeptide according to the invention and a DNA binding domain of a transcription factor such as, for example, Gal4 or LexA. The transformed cells additionally contain a reporter gene, whose promoter contains binding sites for the corresponding DNA binding domains. By transformation of a further expression vector which expresses a second fusion protein from a known or unknown polypeptide having an activation domain, for example of Gal4 or Herpes simplex virus VP16, the expression of the reporter gene can be greatly increased if the second fusion protein interacts with the polypeptide according to the invention. This increase in expression can be utilized in order to identify novel pharmacologically active substances, for example by preparing a cDNA library from regenerating tissue for the construction of the second fusion protein. Moreover, this test system can be utilized for the screening of substances which inhibit an interaction between the polypeptide according to the invention and pharmacologically active substance. Such substances decrease the expression of the reporter gene in cells which express fusion proteins of the polypeptide according to the invention and of the pharmacologically active substance (Vidal and Endoh, 1999, Trends in Biotechnology; 17: 374-81). Novel active compounds which can be employed for the therapy of disorders of regenerative processes can thus be rapidly identified.

Furthermore a test system may be based on binding a polypeptide useable according to the invention, or a functional variant thereof and/or a nucleic acid coding for this or a variant thereof, and/or an antibody or an antibody fragment directed against a polypeptide useable according to the invention or a functional variant thereof, to a solid phase and test substances for interactions, for example for binding or for changes of the conformation. Suitable systems such as affinity chromatography and fluorescence spectroscopy are known to the person skilled in the art.

Solid-phase bound polypeptides useable according to the invention, or functional variants thereof or nucleic acids coding for these or variants thereof, or antibodies or antibody fragments directed against polypeptides useable according to the invention or functional variants thereof can also be part of an array.

In a preferred embodiment of the invention at least one polypeptide useable according to the invention or a nucleic acid coding for this, or at least one antibody or antibody fragment useable according to the invention may be used in the form of an array fixated to a carrier, for the analysis in connection with diseases of skin cells, of wound healing and/or disorders of wound healing.

Processes for the production of arrays by means of solid-phase chemistry and photolabile protecting groups are known from U.S. Pat. No. 5,744,305. Such arrays can also be brought into contact with substances or libraries of substances in order to test the substances for interactions, for example for binding or for changes of the conformation.

A substance to be tested may for example contain a detectable marker, for example a substance which is radioactively labeled, fluorescence-labeled or luminescence-labeled. Furthermore substances may be coupled to proteins that allow indirect detection, for example by enzymatic catalysis using a peroxidase-assay with a chromogenic substrate or by binding of a detectable antibody. Modifications of the conformation of a polypeptide useable according to the invention can be detected by interaction with a suitable test-substance that for example changes fluorescence of an endogenous tryptophan within the molecule.

Pharmacologically active substances of the polypeptides according to the invention can also be nucleic acids which are isolated by means of selection processes, such as, for example, SELEX (see Jayasena, 1999, Clin. Chem. 45: 1628-50; Klug and Famulok, 1994, M. Mol. Biol. Rep. 20: 97-107; Toole et al., 1996, U.S. Pat. No. 5,582,981). In the SELEX process, typically those molecules which bind to a polypeptide with high affinity (aptamers) are isolated by repeated amplification and selection from a large pool of different, single-stranded RNA molecules. Aptamers can also be synthesized and selected in their enantiomorphic form, for example as the L-ribonucleotide (Nolte et al., 1996, Nat. Biotechnol. 14: 1116-9; Klussmann et al., 1996, Nat. Biotechnol. 14: 1112-5). Thus isolated forms have the advantage that they are not degraded by naturally occurring ribonucleases and therefore have greater stability.

In a preferred embodiment of the invention, a test for identifying pharmacologically active substances is used, where candidate substances are tested for their influence on the expression of at least one nucleic acid useable according to the invention.

Assays for the identification of pharmacologically active substances, which influence the expression of genes are known to the person skilled in the art (see for example Sivaraja et al., 2001, U.S. Pat. No. 6,183,956).

It is possible for example to cultivate cells, which express nucleic acids useable according to the invention, for example HeLa cells as a test system for the analysis of gene expression in vitro. Preferably the cells are skin cells, even more preferably they are keratinocytes, fibroblasts or endothelial cells. A possible test system constitutes the human keratinocyte cell line HaCat which is generally available.

The analysis of gene expression can be performed for example on the mRNA or protein level. Here, the amount of nucleic acid or protein useable according to the invention is measured after the application of one or more candidate substances to the cell culture and is then compared with the amount in a control cell culture. This can be performed for example by hybdridization of an antisense probe which can be used to detect mRNA of target genes useable according to the invention in cell lysates. Quantification can be performed for example by binding of a specific antibody to the mRNA-probe complex (see Stuart and Frank, 1998, U.S. Pat. No. 4,732,847). It is possible to perform the analysis as a high-throughput analysis to test a lot of substances with respect to their suitability as modulator of gene expression of nucleic acids useable according to the invention (Sivaraja et al., 2001, U.S. Pat. No. 6,183,956). The substances to be analyzed can be taken from substance libraries (see for example. DE19816414, DE19619373) which contain several thousand, often very heterogeneous substances. Alternatively, the total RNA or mRNA can be isolated from cells and subsequently the absolute or relative amount of mRNA of a target gene useable according to the invention can be determined for example by the use of quantitative RT-PCR (see EP 0 200 362; Wittwer et al., 1997, BioTechniques 22:130-8; Morrison et al., 1998, BioTechniques 24: 954-62) or RNAse Protection Assays (see for example Sambrook et al., 1989, Molecular cloning: A Laboratory Manual, Cold Spring Harbor, Cold Spring Harbor Laboratory Press, New York, chapter 7; EP 0 063 879). Another possibility constitutes the detection of the amount of protein in cell lysate by the use of an antibody which specifically detects the protein useable according to the invention. The quantification can for example be performed by the use of an ELISA or Western blot analysis, which are generally known to a person skilled in the art. To determine the specificity of the substances for the expression of nucleic acids useable according to the invention, the influence of the candidate substances on the target gene expression can be compared to the influence on the expression on other genes, for example genes of the cell metabolism like GAPDH. This can be performed in separately or in parallel to the analysis of the nucleic acids useable according to the invention.

The pharmacologically active substances identified with the aid of the test procedures useable according to the invention can be used, if appropriate combined or together with suitable additives and/or auxiliaries, for the production of a diagnostic or medicament for the diagnosis, prevention and/or treatment of diseases of skin cells, of wound healing and/or their pathological disorders.

A further subject of the invention relates to the use of at least one polypeptide useable according to the invention or of a functional variant thereof and/or of a nucleic acid encoding this or a variant thereof, and/or of an antibody or an antibody fragment directed against a polypeptide useable according to the invention or a functional variant thereof, if appropriate combined or together with suitable additives and/or auxiliaries, for the production of an array attached to a carrier material for analysis in connection with diseases of skin cells and/or of wound healing and/or their pathological disorders.

Processes for preparing such arrays are known, for example, from U.S. Pat. No. 5,744,305 by means of solid-phase chemistry and photolabile protective groups.

The present invention furthermore relates to the use of at least one polypeptide useable according to the invention or a functional variant thereof and/or of a nucleic acid encoding this or a variant thereof, and/or of an antibody or an antibody fragment directed against a polypeptide useable according to the invention or a functional variant thereof, if appropriate combined or together with suitable additives and/or auxiliaries, in the form of an array for analysis in connection with diseases of skin cells, in wound healing and/or their pathological disorders.

For analysis in connection with diseases of skin cells and/or of wound healing and/or their pathological disorders, it is also possible to use, for example, DNA chips and/or protein chips which comprise at least one nucleic acid, at least one polypeptide, and/or at least one antibody or antibody fragment, as described above. DNA chips are disclosed, for example, in U.S. Pat. No. 5,837,832.

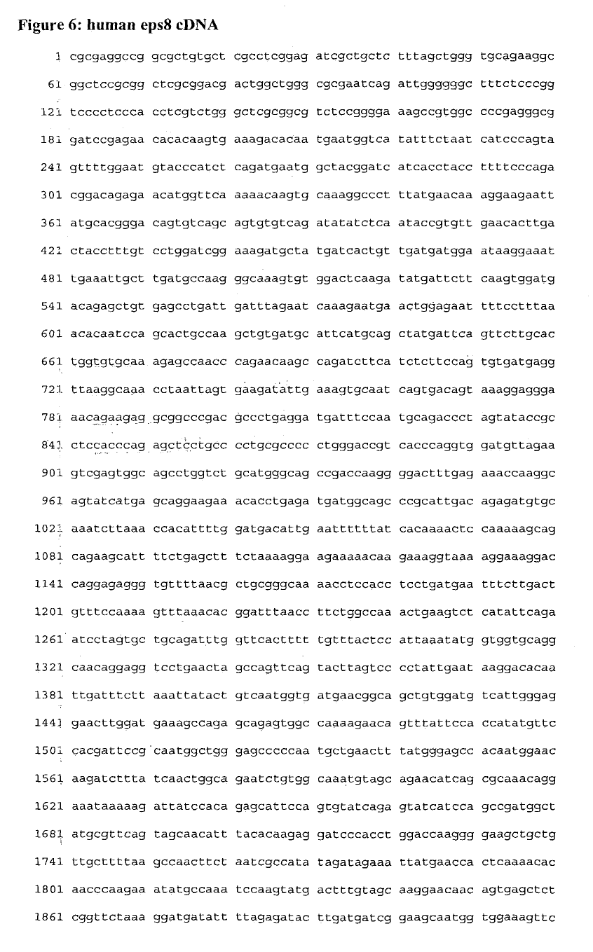

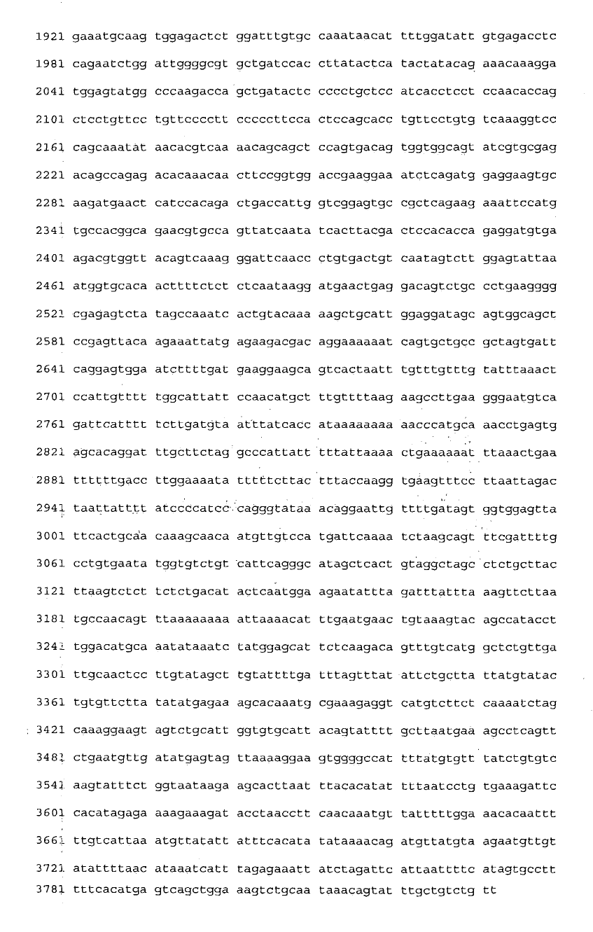

In particular, the polypeptide of the invention is the polypeptide Eps8 and this polypeptide is encoded by the nucleic acids depicted in FIGS. 5 and 6. Further, the nucleic acid sequence of eps8 is also shown in SEQ ID NOS: 85 and 86.

The nucleic acid can be inserted into a vector. For the use in gene therapy the use of an expression vector is advantageous. The expression vector, which allows the expression of the Eps8 polypeptide, contains an expression construct, which comprises in 5′ to 3′ direction (i) a promoter, (ii) the nucleic acid, in particular a nucleic acid coding for an Eps8 polypeptide, and (iii) a poly A sequence for the correct termination of the Eps8 mRNA.

The promoter preferably allows constitutive expression, regulatable expression, tissue-specific expression, cell-cycle-specific expression, cell type-specific expression and/or metabolically-specific expression. When the nucleic acid is eps8, it is preferable to use a skin-specific promoter.

The vector may be a viral vector and the virus, from which the vector is derived, is selected from the group consisting of baculoviruses, vaccinia viruses, adenoviruses, adeno-associated viruses, retroviruses and herpesviruses. Among this list, baculovirus-derived vectors have a particular advantage as baculoviruses are easy to handle and the amount of expressed polypeptide is usually very high.

Further, adenoviruses are preferred and if they are used, the adenovirus comprises a sequence derived from adenovirus type 2 and/or 5. It is also advantageous to delete sequences which are essential for the replication of the adenovirus. In the expression construct at least one adenoviral region is replaced by the expression construct. This region is selected from the group consisting of the E1 region, the E3 region and the region downsteam of the E4 region. The adenovirus vector may also have a deletion in more than one of these regions.

The vector may also be a non-viral vector and this vector may be selected from the group consisting of a virosome, a liposome, a naked DNA, and a poly-lysine-conjugated DNA. Further, the liposome may contain DOTMA, DPOE, DOTAP or DOGS.

For the use in therapy, the polypeptide, the nucleic acid, the antibody and/or the cell should be formulated in a pharmaceutically acceptable carrier. Depending on the particular application, any pharmaceutically acceptable carrier known in the art may be used.

The polypeptide, the nucleic acid, the antibody and/or the cell may be administered by different application techniques among which topical or systemic administration, local injection, intradermal administration and subcutaneous administration are the most common. If the polypeptide or the nucleic acid is Eps8 or the nucleic acid coding for the Eps8 polypeptide, topical administration, direct local injection into a wound of the patient's skin, intradermal or subcutaneous administration is of particular advantage.

If a wound healing disorder is treated, the disorder is selected from the group consisting of a wound infected with a microorganism, a wound infected with a virus, an ischemic wound, a scar and a wound from a patient suffering from a condition selected from the group consisting of diabetes, alcoholism, arterial disorder and venous insufficiency. Most preferred is the treatment of a wound of a patient suffering from diabetes.

The wound can also be a badly healing wound and such wounds are selected from the group consisting of a diabetic wound, a diabetic ulcer (“diabetic foot”), a neuropathic wound, a venous ulcer, and an arterial ulcer.

The skin disorder can be a disorder selected from the group consisting of psoriasis, eczema, acne, urticaria, atopic dermatitis, defective skin pigmentation, in particular vitiligo, defective hair growth, defective hair metabolism and senile skin.

The invention will now be further illustrated below with the aid of the figures and examples, without the invention being restricted thereto.

Description of the Tables, Figures and Sequences:

Table 1: Tabulation of the differential expression of various genes relevant for wound healing in wounds of 10 weeks old BALB/c mice and in wounds of young (4 weeks of age) and old (12 months) mice, as well as in intact skin and wounds of dexamethasone-treated, badly healing wounds and of control mice.

Table 2: Tabulation of the differential expression of various genes that are relevant for wound healing in intact skin and in wounds of mice with diabetes and of control mice.

Table 3: Tabular survey of polypeptide sequences with unknown biological function that were identified during the analysis of gene expression during wound healing, and their cDNAs and accession numbers or SEQ ID numbers.

Table 4: Tabular survey of the polypeptide sequences with already known and described functions identified in the analysis of gene expression during the wound healing process and their cDNAs and accession numbers or SEQ ID numbers.

Table 5: Tabular survey of the polypeptide sequences with already known and described functions that were additionally identified in the analysis of gene expression during the wound-healing process, and their cDNAs and accession numbers or SEQ ID numbers.

Table 6: Analysis of the kinetics of wound-relevant genes during wound healing in the mouse by means of “TaqMan analysis”.

Table 7: Analysis of the kinetics of wound-relevant genes during wound healing in humans relative to Cyclophilin by means of “TaqMan analysis”.

Table 8: Analysis of the expression of genes useable according to the invention in the wound ground and the wound edge relative to intact skin of ulcer patients.

Table 9: Analysis of the expression of genes useable according to the invention in intact skin of healthy persons, as well as in lesional and non-lesional skin of psoriasis patients.

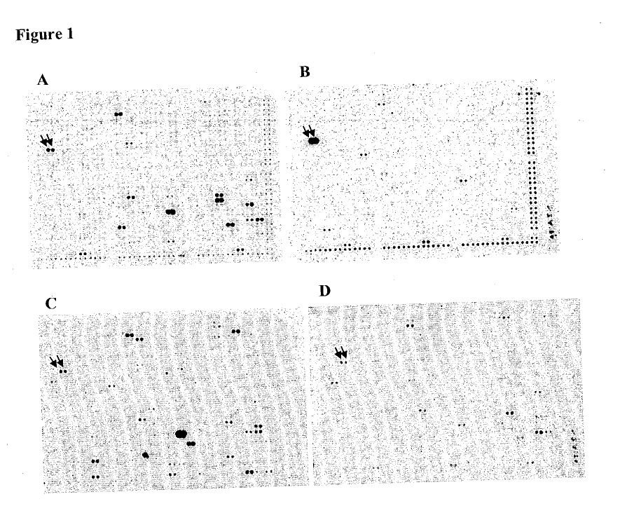

FIG. 1: Autoradiograms of hybridizations of membranes (mouse ATLAS Array, Clontech Laboratories GmbH, Heidelberg) with an identical pattern of applied cDNA fragments using four different samples. All samples were prepared from cDNAs which originated from subtractive hybridizations. A: wound-specific sample (subtraction wound versus intact skin), B: skin-specific sample (subtraction intact skin versus wound), C: sample specific for poorly healing wounds (subtraction wound dexamethasone-treated animals versus wound control animals), D: sample specific for well-healing wounds (subtraction wound control animals versus wound dexamethasone-treated animals). The positions of the TTF-I cDNAs (each loaded twice) are indicated with arrows.

FIG. 2: Comparison of the polypeptide sequences of the identified proteins of SW1136 from mouse (murine) and human. Differences to the human sequence of SW1136 are indicated.

FIG. 3: Comparison of the polypeptide sequences of the identified proteins of SW1295 from mouse (murine) and human. Differences to the human sequence of SW1295 are indicated.

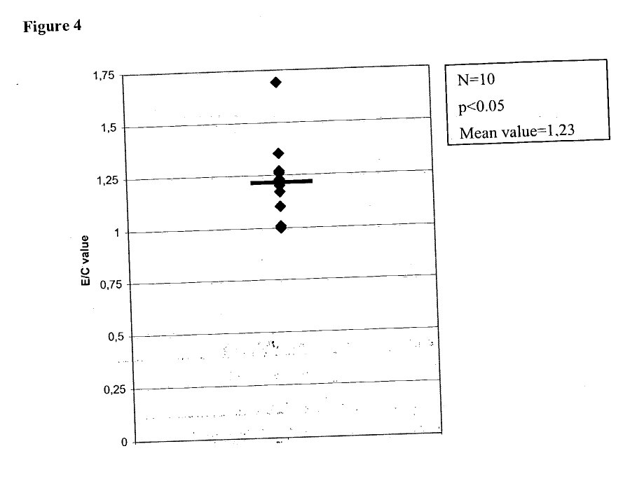

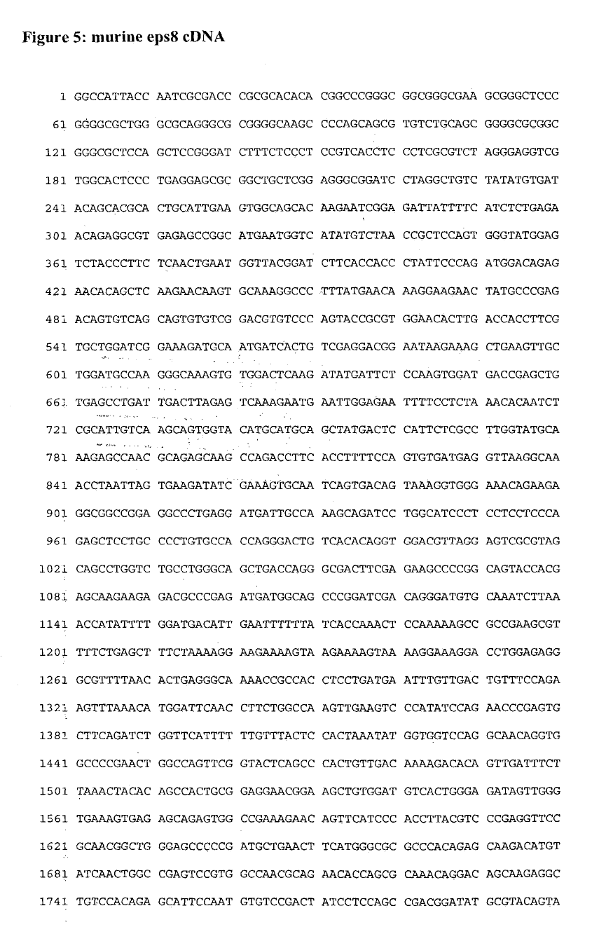

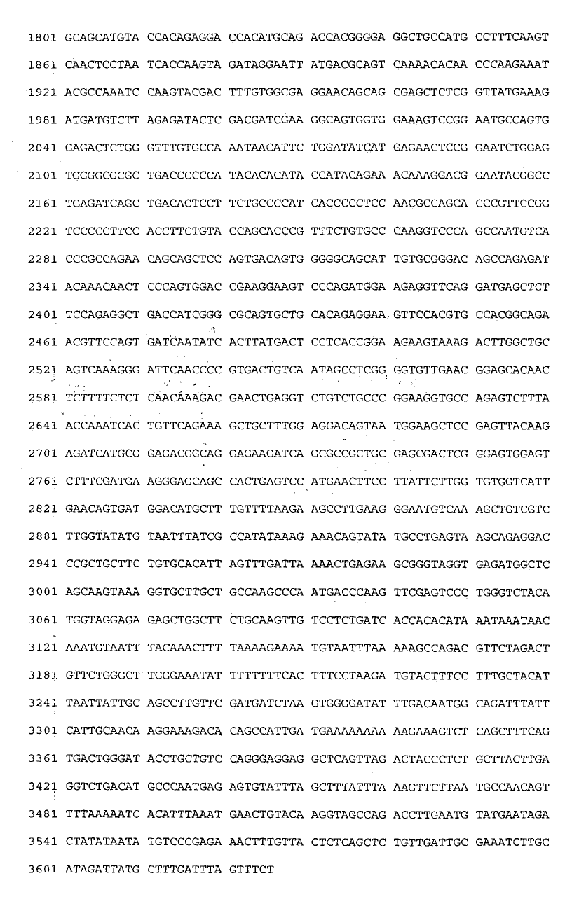

FIG. 4: Use of eps8 nucleic acid for improvement of wound healing and results of example 8.

FIG. 5: Murine eps8 cDNA.

FIG. 6: Human eps 8 cDNA.

SEQ ID No. 1 to SEQ ID No. 58 and SEQ ID No. 63 to SEQ ID No. 73 and SEQ ID No. 80 to SEQ ID No. 84 show the polypeptide or cDNA sequences useable according to the invention from human or mouse.

SEQ ID No. 59 to SEQ ID No. 62 and SEQ ID No. 74 to SEQ ID No. 79 show DNA sequences of oligonucleotides, while SEQ ID NOS. 85 and 86 show DNA sequences of polynucleotides which were used for the experiments of the present invention.

### EXAMPLES

### Example 1

### Enrichment of Wound-Relevant cDNA by Means of Subtractive Hybridization and Identification of TTF-I as Wound-Relevant Gene

Total RNA was isolated from intact skin and from wound tissue (wounding on the back 1 day before tissue sampling by scissors cut) of BALB/c mice by standard methods (Chomczynski and Sacchi, 1987, Anal. Biochem. 162: 156-159, Chomczynski and Mackey, 1995, Anal. Biochem. 225: 163-164). In order to obtain tissue of mice with poorly healing wounds, BALB/c mice were treated before wounding with dexamethasone (injection of 0.5 mg of dexamethasone in isotonic saline solution per kg of body weight twice per day for 5 days). The RNAs were then transcribed into cDNA with the aid of a reverse transcriptase. The cDNA synthesis was carried out using the “SMART PCR cDNA synthesis kit” from Clontech Laboratories GmbH, Heidelberg, according to the directions of the corresponding manual.

In order to identify those cDNAs which occurred with differing frequency in the cDNA pools, a subtractive hybridization (Diatchenko et al., 1996, Proc. Natl. Acad. Sci. U.S.A. 93: 6025-30) was carried out. This was effected using the “PCR select cDNA subtraction kit” from Clontech Laboratories GmbH, Heidelberg, according to the directions of the corresponding manual, the removal of excess oligonucleotides after the cDNA synthesis being carried out by means of agarose gel electrophoresis. Four cDNA pools were set up, which were enriched for wound-relevant genes, where one pool was enriched for cDNA fragments which are expressed more strongly in the wound tissue in comparison with intact skin (“wound-specific cDNA pool”), one pool was enriched in cDNA fragments which are more strongly expressed in intact skin in comparison with wound tissue (“skin-specific cDNA pool”), one pool was enriched in cDNA fragments which are more strongly expressed in well-healing wounds in comparison with poorly healing wounds (“well healing cDNA pool”) and one pool was enriched in cDNA fragments which are more strongly expressed in poorly healing wounds in comparison with well-healing wounds (“poorly healing cDNA pool”).

In order to identify those genes which were contained in the cDNA pools relevant to wound healing, the presence of the corresponding cDNAs in the pools was analyzed in “reverse Northern blot”. Here, the cDNA fragments are immobilized on membranes in the form of arrays of many different cDNAs, and hybridized with a complex mixture of radio-labeled cDNA (Sambrook et al., 1989, Molecular Cloning: A Laboratory Manual, Cold Spring Harbor, Cold Spring Harbor Laboratory Press, New York, Chapter 9 page 9.47 to 9.58 and Chapter 10 page 10.38 to 10.50; Anderson and Young: Quantitative filter hybridization; in: Nucleic Acids Hybridisation, A Practical Approach, 1985, Eds. Hames and Higgins, IRL Press Ltd.; Oxford, Chapter 4, page 73 to 112). For example, commercially available membranes were used (mouse ATLAS array, Clontech).

For the preparation of suitable hybridization samples, the subtracted cDNA pools were treated with the restriction endonuclease RsaI and purified by means of agarose gel electrophoresis (Sambrook et al., supra, Chapter 6, page 6.1 to 6.35) in order to separate the cDNA synthesis and amplification primer (see manual “PCR-Select cDNA Subtraction Kit”, Clontech). The cDNAs were then radio-labeled using the “random hexamer priming” method (Feinberg and Vogelstein, 1983, Anal. Biochem. 132: 6-13) in order to prepare hybridization samples.

The membrane was preincubated in 25 ml of hybridization solution for 30 min at 65° C. (25 mM sodium phosphate, pH=7.5, 125 mM NaCl, 7% SDS). The hybridization sample was denatured at 100° C. for 10 min, then cooled on ice, about 100 CPM (“counts per minute”) per ml were added to the hybridization solution and the hybridization was carried out in a hybridization oven for 16 hours at 65° C. The membrane was then washed twice with the hybridization solution without sample at 65° C. for 10 min. The membrane was then washed at 65° C. a number of times for 10 min in each case in wash solution (2.5 mM sodium phosphate, pH=7.5, 12.5 mM NaCl, 0.7% SDS) until it was no longer possible to detect any activity in the solution poured off. The radioactive signals were analyzed using a phosphoimager (BioRad, Quantity One®) (FIG. 1). Those cDNAs were then selected which produced different signal intensities with the various samples. This resulted at the position of TTF-I on the membrane, in a significantly stronger signal intensity with the skin specific cDNA pool in comparison to the wound specific cDNA pool (FIG. 1A, B). The analysis of the experiment, in which hybridization was performed in parallel with the “poorly healing” cDNA pool and the “well healing” cDNA pool, showed, that the hybridization sample of the “poorly healing” cDNA pool hybridized significantly stronger at the position of TTF-I (FIG. 1C, D). Therefore, differential expression of TTF-I was observed at two different wound healing states.

### Example 2

### Verification of the Expression Pattern of TTF-I by Means of “Real-Time Quantitative RTPCR”

A verification of the differential expression of the nucleic acids described above as well as the investigation of further wound healing states was carried out by real-time RTPCR in the ABI Prism 7700 sequence detection system (PE Applied Biosystems). The apparatus was equipped with the ABI Prism 7200/7700 SDS software version 1.6.3 (1998). The detection of PCR products was carried out during the amplification of the cDNA with the aid of the stain SYBR Green 1, whose fluorescence is greatly increased by binding to double-stranded DNA (Karlsen et al. 1995, J. Virol. Methods. 55: 153-6; Wittwer et al., 1997, BioTechniques 22: 130-8, Morrison et al., 1998, BioTechniques 24: 954-62). The basis for the quantification is the PCR cycle (threshold cycle, CT value) which is reached when the fluorescence signal exceeds a defined threshold. The analysis is carried out by means of the A-CT method (User Bulletin #2, Relative Quantitation of Gene Expression, PE Applied Biosystems, 1997). The abundances of the cDNAs were determined relative to an endogenous reference (GAPDH). The results are shown in Tables 1 and 2.

To obtain tissue from mice with poorly healing wounds, BALB/c mice were treated prior to wounding with dexamethasone (injection of 0.5 mg dexamethasone in isotonic salt solution per kg body weight twice a day for 5 days). To obtain wound tissue from young and old mice, day 1 wounds from 4 weeks and 12 months old BALB/c mice were employed. To obtain wound tissue from mice with diabetes, day 1 wounds of 10 weeks old C57-BL/Ks-db/db/OLa mice were used. Total RNA was obtained from skin and wound tissue as described above and 1 μg of total RNA was subjected to reverse transcription in a thermocycler (GeneAmp PCR system 9700, Perkin Elmer) using the TaqMan reverse transcription reagent kit (Perkin Elmer) according to the recommendations of the manufacturer (SYBR Green PCR and RT-PCR Reagents Protocol, PE Applied Biosystems, 1998). The primers for the amplification of the TTF-I cDNA (TTF-I-Primer 1: CGAGCGCTACATTGTCGCT (SEQ ID No. 59), TTF-I-Primer 2: GTCTTAAATTTGCTTGTGCCCC (SEQ ID No. 60)) and the reference (GAPDH primer 1: ATCAACGGGAAGCCCATCA (SEQ ID No. 61), GAPDH primer 2: GACATACTCAGCACCGGCCT (SEQ ID No. 62)) were selected with the aid of the Primer Express software for Macintosh PC Version 1.0 (PE Applied Biosystems, P/N 402089, 1998) based on the nucleic acid described above and the known sequence of GAPDH. For the PCR, the SYBR Green PCR Core Reagents Kit (4304886, PE Applied Biosystems) was used. The concentration of the primers in the PCR was initially optimized in the range from 50 nM to 600 nM and the specificity of the PCR was tested by analysis of the length of the amplified products in an agarose gel electrophoresis. The efficiency of the PCR system was then determined by means of a dilution series (User Bulletin #2, Relative Quantitation of Gene Expression, PE Applied Biosystems, 1997). It became apparent that for both cDNAs the efficiency of the amplification was 100%, i.e. at each 1:2 dilution of the cDNA one more cycle was needed in order to exceed the fluorescence threshold value.

For the quantification, each batch of cDNA was amplified from 10 ng of reverse-transcribed total RNA in a total volume of 25 μl. The running conditions for the PCR corresponded to the details of the manufacturer (PE Applied Biosystems, SYBR Green® PCR and RT-PCR Reagents Protocol, 1998). The CT values were analyzed and the abundance of TTF-I relative to GAPDH was calculated. On the one hand the decrease of TTF-I in wounds in comparison to intact skin from control animals as well as of young mice was confirmed (Table 1, compare to FIGS. 1A, B). On the other hand again an increase of expression of TTF-I was observed in poorly healing wounds of dexamethasone treated mice in comparison to well healing wounds of control animals (Table 1, compare to FIGS. 1 B, C). In addition a decrease of TTF-I could be measured in mice with diabetes (Table 2). Therefore, the regulation of expression of TTF-I in different wound healing states was verified.

### Example 3

### Identification of MCP-2 as Gene Relevant for Wounds Using Comparison Counting of Clones in cDNA Libraries Through the Analysis of Restriction Fragment Patterns

BALB/c mice were treated with dexamethasone (injection of 0.5 mg dexamethasone per kg body weight twice a day for 5 days) and subsequently wounded to obtain tissue of poorly healing day 1 wounds. The generation of cDNA of the total RNA isolated from the tissue as well as all further steps was performed as described in EP 0 965 642: the cDNA was cloned in defined orientation in a suitable vector and the thus obtained plasmid was transformed into a suitable E. coli strain. 100 E. coli clones respectively were mixed, and the plasmid DNA was isolated. The DNA was separated into three portions and subsequently hydrolized with the restriction endonuclease BglI and labeled with a fluorescent dye. Subsequently each portion was divided into two portions and then hydrolyzed with one of the restriction endonuclease BfaI, DpnI, RsaI, DdeI, AluI and HinfI. The portioned nucleic acids were separated electrophoretically and the pattern of the separated nucleic acids was analyzed. The restriction fragment pattern of clone mixture was compared with the patterns of single cDNA clone analysis and thus the cDNAs were identified. Those nucleic acids were selected which restriction fragment pattern was identified with a different abundance in cDNA pools of poorly healing and well healing wounds. During the analysis of 37000 cDNAs the pattern of MCP-2 (DdeI:196.23±0.3 base pairs; AluI: 67.51±0.5 base pairs; HinfI: 349.88±0.7 base pairs; BfaI: 531.02±2.0 base pairs; DpnI: 245.02±0.3 base pairs; RasI: 254.23±0.3 base pairs) was identified three times in the cDNA pool of poorly healing wounds, while the pattern was not observed in the cDNA pool of well healing wounds.

### Example 4

### Analysis of the Kinetics of Wound-Relevant Genes During Wound Healing in the Mouse by Means of “TaqMan analysis”

The kinetics of the regulation of the expression of wound-relevant genes, during normal wound healing in the mouse was investigated by means of “TaqMan analysis” using the GeneAmp5700 from Applied Biosystems. Normally healing wound biopsies from various time points after wounding and intact skin were obtained from 610 week-old BALB/c mice treated with isotonic saline solution by scissors cut as described in Example 1. The RNA was isolated by homogenizing the biopsies in RNA clean buffer (AGS, Heidelberg), to which 1/100 part by volume of 2-mercaptoethanol had been added using a disperser. The RNA was then extracted by treating with phenol twice by means of acidic phenol saturated with water and extracted in the presence of 1-bromo-3-chloropropane. An isopropanol and an ethanol precipitation were then carried out and the RNA was washed with 75% ethanol. After this, a DNase I digestion of the RNA was carried out. For this, 20 μg of RNA (to 50 μl with DEPC-treated water) were incubated at 37° C. for 20 min with 5.7 μl of transcription buffer (Roche), 1 μl of RNase inhibitor (Roche; 40 U/μl) and 1 μl of DNase I (Roche; 10 U/μl). 1 μl of DNase I was then added again and the mixture was incubated at 37° C. for a further 20 min. The RNA was then treated with phenol, ethanol-precipitated and washed. All above mentioned steps were carried out using DEPC (diethyl pyrocarbonate)-treated solutions or liquids containing no reactive amino groups. cDNA was then prepared from the extracted RNA. This was carried out in the presence of 1×TaqMan RT buffer (Applied Biosystems), 5.5 mM MgCl₂ (Perkin Elmer), 500 μM each of dNTPs (Perkin Elmer), 2.5 μM of random hexamers (Perkin Elmer), 1.25 U/μl of MultiScribe Reverse Transcriptase (50 U/μl, Perkin Elmer), 0.4 U/μl RNase inhibitor (20 U/μl, Perkin Elmer), 20 μl of RNA (50 ng/μl) and DEPC-treated water (to 100 μl volume). After addition of the RNA and thorough mixing, the solution was divided in 20.2 ml wells (50 μl each) and the reverse transcription was carried out in a thermocycler (10 min at 25° C.; 30 min at 48° C. and 5 min at 95° C.). The cDNA was subsequently quantified by means of quantitative PCR using SYBR green PCR master mixes (Perkin Elmer), a triplicate determination (in each case with target primers and GAPDH primers) being carried out for each cDNA species to be determined. The stock solution for each triplet contained, in a total volume of 57 μl, 37.5 μl of 2×SYBR master mix, 0.75 μl of AmpErase UNG (1 U/μl) and 18.75 μl of DEPC-treated water. Per triplicate determination, 1.5 μL each of forward and reverse primer were added to 57 μl of stock solution in a previously optimized concentration ratio. 60 μl each of the stock solution/primer mixture were mixed with 15 μl of cDNA solution (2 ng/μl) and subdivided into 3 reaction wells. Parallel to this, a stock solution with primers was prepared as a reference for the determination of GAPDH (SEQ ID No. 61 and SEQ ID No. 62), mixed with a further 15 μl of the same cDNA solution and sub-divided into 3 reaction wells. In addition, in order to set up a standard curve for the GAPDH-PCR, various cDNA solutions were prepared as a dilution series (4 ng/μl; 2 ng/μl; 1 ng/μl; 0.5 ng/μl and 0.25 ng/μl). 15 μl each of these cDNA solutions were mixed with 60 μl of stock solution/primer mixture for the determination of GAPDH and subdivided into 3 reaction wells. Likewise, a standard curve for the PCR of the genes to be investigated was set up in each case; the same dilutions which were also employed for the GAPDH standard curve were used here. The control used was a PCR batch without cDNA. 15 μl each of DEPC water were added to 60 μl in each case of stock solution/primer mixture of target and GAPDH in each case, mixed and in each case subdivided into 3 reaction wells. The amplification of the batches was carried out in the GeneAmp 5700 (2 min at 50° C.; 10 min at 95° C., followed by 3 cycles of 15 sec at 96° C. and 2 min at 60° C.; then 37 cycles of 15 sec at 95° C. and 1 min at 60° C.). The analysis was carried out by the determination of the relative abundance of each target gene with respect to the GAPDH reference. For this, a standard curve was first set up by plotting the CT values of the dilution series against the logarithm of the amount of cDNA in the PCR batch (ng of transcribed RNA) and the slope(s) (s) of the straight lines was determined. The efficiency (E) of the PCR then results as follows: E=10^{-1/s}−1. The relative abundance (X) of the cDNA species (Y) investigated in relation to GAPDH is then: X=(1+_{GAPDH})^{C}_{T}^{(GAPDH)}/(1+E_{Y})^{C}_{T}^{(Y)}. The numerical values were then standardized by setting the amount of cDNA from intact skin of the 10 week-old BALB/c control animals equal to 1. The relative changes in the target gene expression in various wound healing states are compiled in Table 6. Thus it is clear, for example, in the case of the tyrosine kinase Fer that the expression of skin disease-relevant targets is specifically regulated during wound healing. In this target, even one hour after wounding, a greatly decreased expression of mRNA occurs which lasts during the entire observation period of 14 d. This shows that differential regulation over the entire period of wound healing is essential for the normal course of wound healing.

A similar kinetic, i.e. a reduced expression upon wounding that lasted for days was observed in a plurality of genes, for example for KIAA1247, Cystatin C, SW1136, SW1295, Baf57, TSC-22, Split hand foot deleted 1, Nicotinamid N-Methyl-Transferase, UBC9, tsg101, HMG-14, TAK1 and Golgi-4-transmembrane spanning transporter. This demonstrates that differential regulation of these genes is required over the entire period of wound healing. However complex kinetics have also been observed, for example phospholipase inhibitor exhibited an upregulation of the expression 1 h after wounding as well as 7 and 14 days after wounding. This clearly shows, that the differential expression of phospholipase inhibitor can lead to disorders of the wound healing process directly after wounding as well as during later phases of wound healing and that disorders of the expression and/or activity of phospholipase inhibitor may lead to disorders of the wound healing directly after wounding as well as in later phases of wound healing.

### Example 5

### Differential Expression of Wound-Relevant Genes in Human Wounds

With the aid of the normally healing wounds, it should now be investigated whether a differential regulation of the expression of the genes identified as wound-relevant verified in Example 4 can also be observed in humans. For this, 4 mm biopsies of intact skin were taken from 6 patients as described above, and also 6 mm biopsies from the same patients at the time points T=1 h, 1 d, 5 d and 14 d. The biopsies of a given time point were pooled and the cDNA was isolated as described above. Then the quantification was done by means of TaqMan analysis as described above, except that the abundance of the target species to be determined was determined relative to cyclophilin (EMBL: Y00052). The primers 10 used for this are cyclophilin primer 1: TCTTAACCAC CAGATCATTC CTTCT (SEQ ID No. 78) and cyclophilin primer 2: GGATACTGCG AGCAAATGGG (SEQ ID No. 79). The analysis of the experiment is shown in Table 7. In the case of CCR-1 (CCR-1 Primer 1: CCCAATGGGA ATTCACTCAC C (SEQ ID Nr. 76); CCR-1 Primer 2: GCTTCCACTC TCGTAGGCTT TC (SEQ ID Nr. 77)), a strong increase in CCR-1 expression until 24 h after wounding followed by a slow decrease in CCR-1 expression was observed in human. This is fully consistent with the kinetics of CCR-1 expression in murine wound biopsies (Table 6). Also, it was possible to show, for example, in the case of the Golgi 4 transmembrane spanning transporter that differential regulation of expression during wound healing can be detected both in the mouse and in man. Thereby, it was thus possible to verify the relevance of the target to wound healing and skin diseases. Also the other genes analyzed showed a differential regulation upon wounding. Thus it was possible to demonstrate that the targets are relevant for wound healing and for skin disorders. The genes showed complex kinetics of expression with transient changes, such as for example in the case of Eps8, Phospholipase Inhibitor, TSC-22, Cathepsin C and HMG-14 as well as a steady increase in expression spanning the time of investigation such as in the case of KIAA1247 and Cystatin C. This demonstrates, that the precise regulation of the expression and/or activity of the targets is essential for the normal course of the wound healing process both in mice and humans.

### Example 6

### Differential Expression of Wound-Relevant Genes in Human Ulcers

In order to show that the genes identified as wound-relevant are differentially regulated not only in normally proceeding wound healing but also in the case of a wound-healing disorder, biopsies from patients having chronic venous ulcers were taken at the same time from intact skin and from the wound ground and the wound edge and were investigated for expression of the target genes. From each group (intact skin, wound edge, wound ground), the biopsies of 6 subjects in each case were pooled. RNA was isolated from all biopsies as described in Example 4 transcribed into cDNA. The quantification of wound healing-relevant cDNAs was carried out also as described in Example 5, the amount of cyclophilin mRNA being used for the calculation of the relative amount of the target gene cDNA. The results of the experiments are compiled in Table 8. Thus, in the case of KIAA1247, for example, a dysregulation of the expression in the ulcers was found in comparison to the normally healing wound (Table 7): while in the normally healing wound a steady increase in the KIAA1247 was observed, a markedly reduced expression was found at the wound edge. This shows that the differential expression of KIAA1247 is essential not only for wound healing, but that dysregulations can lead to severe wound-healing disorders. The experiment illustrates that KIAA1247 can be used for the diagnosis, prevention and/or treatment of wound-healing disorders and/or skin diseases.

### Example 7

### Differential Regulation of Genes Useable According to the Invention in Lesional and Non-Lesional Skin of Psoriasis Patients in Comparison with Intact Skin of Healthy Patients

It should now be verified with the aid of psoriasis patients that genes useable according to the invention play an important part not only in wound healing and wound-healing disorders but also in other skin diseases. For this, 4 mm punch biopsies both of lesional and non-lesional skin were taken from psoriasis patients as described in Example 5. As a control, biopsies of intact skin were taken from healthy subjects. The isolation of the mRNA from the individual biopsies was carried out by embedding the biopsies in tissue freezing medium (Jung), the reduction of the biopsy into pieces using a microtome and the subsequent mRNA isolation by means of Dynabeads-Oligo dT (Dynal). The hackled biopsies were first suspended in lysis-buffer and then homogenized using the Polytron. In order to fragment the genomic DNA, the solution is centrifuged through Qia-Shredder columns and additionally sheared a number of times in a syringe with a needle. The Dynabeads were pretreated according to the instructions of the manufacturer and mixed with the lysis homogenate (250 μl of the stock suspension), incubated and washed (final volume 250 μl). The suspension was then divided into one portion each of 240 μl and of 10 μl (as a control). For the first strand synthesis, the following components were mixed: 20 μl of 10×TaqMan RT buffer, 44 μl of 25 mM MgCl₂, 40 μl of dNTP mix (2.5 mM/dNTP), 87 μl of DEPC-H₂O, 4 μl of RNase inhibitor (20 U/μl) and 5 μl of MultiScribe transcriptase (50 U/μl). 195 μl of the reaction mix were then added to the 240 μl batch and 20 μl to the control batch, mixed and incubated at 48° C. for 45 min. The Dynabeads were then pelleted in a magnetic particle collector and the supernatant was withdrawn. 20 μl of Tris-HCl buffer were then added and the suspension was incubated at 95° C. for 1 min. The Dynabeads were immediately pelleted in a magnetic particle collector and the mRNA in the supernatant was withdrawn. The cDNA/Dynabeads were then washed 3× with TE buffer. For the second strand synthesis, the cDNA/Dynabeads were washed 2× in 1× EcoPol buffer and a solution of the following components was added: 23 μl of lox EcoPol buffer; 4.6 μl of dNTP mix (25 mM/dNTP); 11.5 μl of random hexamers; 118.7 μl of DEPC-H₂O. The suspension was mixed briefly with the aid of a vortexer and 9.2 μl of Klenow fragment (5 U/μl) were then added. 200 μl of this solution were added to the batch, 20 μl to the control batch, and the suspensions were incubated at 37° C. for 1 h. The DNA was then melted at 94° C. for 1 min and the Dynabeads were pelleted in a magnetic particle collector. The supernatant was transferred to a new reaction vessel and the enzyme was inactivated at 75° C. for 10 mm. The sense DNA strands contained in the supernatant were then employed for the TaqMan analysis.

The TaqMan analysis was carried out as described in Example 5, the amount of GAPDH (hGAPDH-Primer 1: CCTCCCCTCTTCAAGGGTCTA (SEQ ID Nr. 74); hGAPDH-Primer 2: AGGAGTAAGACCCCTGGACCA (SEQ ID Nr. 75) being used for the calculation of the relative abundance of the respective mRNA species in the individual biopsies. Since a far greater amount of total mRNA is isolatable from the skin biopsies of psoriasis patients, in particular from lesional skin, than from intact skin of healthy subjects, a standardization to identical amounts of mRNA is necessary, the amount of GAPDH mRNA being assumed as a housekeeping gene as a marker for the amount of total mRNA. A total of 4 biopsies of intact skin of healthy subjects were analyzed, and also 8 biopsies in each case of lesional and non-lesional skin from psoriasis patients. The abundances of target gene cDNA in the individual groups (intact skin, lesional skin, non-lesional skin) were then standardized to the total amount of the abundances of the cDNAs measured on a microtiter plate. These analyses were carried out for Eps8 (EMBL: U12535); KIAA1247 (GB: AB033073; WO 99/63088) and MASPIN (EMBL: U04313); the average values of the results are compiled in Table 9. It is clear that in both genes useable according to the invention a clear and statistically significant (p<0.05, paired t-test) reduced regulation in lesional skin of 8 psoriasis patients is observed compared with non-pathological skin of the same patients in each case. This illustrates that a dysregulation of these genes can lead to skin diseases and that the genes useable according to the invention are therefore suitable for the prevention and/or treatment and/or diagnosis of skin diseases. In the case of psoriasis patients, the aim is to modulate, preferably to activate, the expression and/or activity of KIAA1247 and/or Eps8 and/or MASPIN. Modulation in the skin, in particular the lesional skin, of the psoriasis patients is prefered here.

As exemplified by KIAA1247, the relationship between dysregulation of the gene and psoriatic disease should now be demonstrated. Here, the conductivity of the skin (a measure of the moisture of the skin) was determined as a measurement parameter and compared with the KIAA1247 expression of the gene in this part of the skin. The conductivity of the skin was determined using a corneometer according to the instructions of the manufacturer (Courage and Khazaka Electronics). It was found here that a statistically significant positive correlation (p=0.000659, Pearson Product Moment Correlation) is observed between conductivity of the biopsy investigated in each case and the KIAA1247 expression: in very dry biopsies of lesional psoriatic skin having a low conductivity, a correspondingly low KIAA1247 mRNA expression was measured, while in more moist skin, i.e. in non-pathogenic skin of the psoriatic patients and in intact skin of healthy subjects, a markedly stronger KTAA1247 expression is detectable. This verifies the relevance of KIAA1247 expression for the pathogenesis of skin diseases.

### Example 8

### Improvement of Wound Healing in Vivo by Application of eps8 Coding Sequence

In order to prove that Eps8 is especially suitable for the improvement of wound healing and wound healing disorders, the effect of murine Eps8 was tested on incisional wounds in vivo in rats. Therefore, wound healing was measured after application of eps8 coding sequence in male Sprague Dawley rats. For quantification of the wound healing process, the tensile strength of wounds was measured. A higher tensile strength reflects improved wound healing. The sequence of murine eps8 cDNA is shown in FIG. 5 (the coding sequence begins at position 321 and ends at position 2786).

For this experiment a suitable adenoviral expression vector Adeps8 was generated based on the Ad-Easy system (Quantum Biotechnologies). A KpnI/ApaI DNA fragment containing full-length murine eps8 cDNA and HA tag was subcloned into pShuttle-CMV vector (Quantum Biotechnologies, Montreal, Canada), and the resulting plasmid was co-transformed into BJ5381 cells with Ad-Easy-I adenoviral backbone DNA that was E1 and E3 deleted and was replication-deficient. The recombinant adenoviral construct was linearized with EcoRI and transfected into 293A cells to produce viral particles. Adenovirus containing the β-galactosidase transgene (Ad-lacZ) was purchased from Quantum Biotechnologies, which contained the same adenoviral backbone DNA as above. To obtain a large preparation of adenovirus, Ad-eps8 was amplified in 293A cell factories (Nalgene Nunc), purified by cesium chloride centrifugation, and desalted with PD-10 columns (Amersham Pharmacia Biotech, Uppsala, Sweden). The particle number for the adenovirus was determined by absorption at 260 nm using GeneQuant-2 (Pharmacia Biotech) spectrophotometer. The plaque-forming unit (PFU) titers were estimated by overlaying infected 293A cells with 1.25% Sea-Plaque agarose (FMC Bioproducts) following the protocol from Quantum Biotechnologies. The final ratio of particle/PFU was 200:1. 5 rats with a body weight of 250-300 g were subsequently anaesthesized with a mixture of oxygen und 2.5% isoflurane. Four full thickness incisions (1.8 cm) were made in each rat, and 1×10⁸ PFU of either Ad-eps8 or Ad-LacZ in 50 μl of HBS vehicle (20 mM HEPES, 150 mM NaCl, pH 7.8) were injected along the wound margins during the surgery. The skin margins were closed with wound clips. At day 7 after surgery, the tensile strength of the wounds was determined using a BTC-2000 tensiometer (SRLI Technologies, Nashville, Tenn.) according to the manufacturer's statement. The pressure needed to break the wounds was determined as a measure of the tensile strength of wounds. A higher tensile strength reflects improved wound healing. Subsequently, E/C values were determined by dividing the absolute tensile strength treated with Ad-eps8 by the absolute tensile strength of a wound treated with Ad-lacZ on the same animal. Hence, E/C values higher than 1 reflect improved wound healing in wounds treated with eps8. The results are shown in FIG. 4. It is evident from the results that treatment with eps8 by a gene therapeutic approach leads to significantly improved healing (Average E/C value: 1.23; N=10; p<0.05). This underlines the extraordinary suitability of eps8 for treating or promoting wound healing.

Due to the very high homology between human and murine eps8 (88% identity) it is apparent that human eps8 (cDNA shown in FIG. 6; coding sequence from nucleotides 210-2678) is equally efficient in improving wound healing in mammals, including humans.

### Example 9

### Improvement of Wound Healing in Diabetic Rats in Vivo by Application of eps8 Coding Sequence

In order to demonstrate the suitability of eps8 for wound healing in diabetic patients, diabetic rats are used for a further experiment, which is basically conducted as described in example 8. The expression vectors Ad-eps8 and as a control Ad-lacZ are constructed as described in example 8. For the induction of diabetes in test animals, rats with a total weigh of 250-300 g are injected with an freshly prepared aqueous solution of streptozotocine (Sigma), wherein 50 mg/kg body weight are used. 5, 10 and 17 days after injection, the aminals' blood sugar is tested. If the blood sugar level is above 200 mg/dL, the animal is considered to comprise a diabetic condition. The wounds are set on day 10 after injection of streptozotocine. The treatment with oxygene and isoflurane, wound setting, injection of eps8 and wound closing is also conducted as described in example 8.

As an alternative, a suitable expression vector based on the vector pMH (Roche) is generated by introducing intron II of the rat insulin gene between the CMV promoter and the multiple cloning site using the HindIII site, resulting in vector pMHInt. Using the multiple cloning site, eps8 coding sequence is cloned into this modified vector by amplifying the coding sequence of eps8 cDNA by PCR and cleavage with suitable restriction enzymes allowing subsequent in frame ligation with pMHInt, resulting in pMHInt_Eps8. As a control, a pMHInt vector containing a luciferase gene is used (pMHInt_luc).

It is also possible to use gene gun injection instead of direct wound injection. For this, the backs of the animals are shaved and 4 marks are set on the each back for later wounding. Each mark is shot with 0.5 μg plasmid DNA bound to gold particles (BioRad) using the Helios Gene Gun. On each rat, 2 marks (one anterior, one posterior) are shot with pMHInt Eps8, the two remaining with pMHInt_luc. Subsequently, 1 to 1.8 cm long incisional wounds are set through the shot marks and the wounds are closed with wound clips. The measurement of the tensile strength is again carried out as described in example 8.

This example will underline the suitablility of eps8 for the improvement of wound healing in diabetic animals. As human and murine eps8 show a very high homology, human eps8 cDNA will be equally efficient in improving wound healing in diabetic mammals, including humans.

It will be apparent to those skilled in the art that various modifications and variations can be made to the compositions and processes of this invention. Thus, it is intended that the present invention cover such modifications and variations, provided they come within the scope of the appended claims and their equivalents.

Priority application DE 10030149.5-41, filed Jun. 20, 2000, and U.S. 60/222,081, filed Aug. 1, 2000 including the specification, drawings, claims and abstract, is hereby incorporated by reference. All publications cited herein are incorporated in their entireties by reference.

TABLE 1

| | Intact skin | Wound | Intact skin | Wound | Intact skin | Wound | Intact skin | Wound |
|---|---|---|---|---|---|---|---|---|
| Gene differentially expressed | control animals | control animals | dexamethasone | dexamethasone | young mice | young mice | old mice | old mice |
| TTF-1 | 1.00 | 0.81 | 1.00 | 1.31 | 1.87 | 0.78 | 0.84 | 0.65 |
| CCR-1 | 1.00 | 31.84 | 0.45 | 76.74 | 1.07 | 22.22 | 0.84 | 24.18 |
| MASPIN | 1.00 | 0.76 | 1.00 | 1.03 | 2.00 | 0.39 | 1.18 | 0.45 |
| B-Raf | 1.00 | 0.77 | 0.77 | 0.71 | 1.29 | 0.63 | 0.61 | 0.70 |
| Prothymosin alpha | 1.00 | 0.97 | 0.91 | 1.90 | 1.36 | 0.67 | 0.78 | 0.48 |
| Eps8 | 1.00 | 0.70 | 0.60 | 0.20 | 0.80 | 0.40 | 0.60 | 0.60 |
| KIAA1247 | 1.00 | 0.30 | 0.90 | 0.20 | 2.20 | 0.70 | 1.00 | 0.20 |
| Cystatin C | 1.00 | 0.77 | 0.74 | 2.21 | 0.76 | 0.37 | 0.91 | 0.26 |
| SW1136 | 1.00 | 0.83 | 0.58 | 1.43 | 1.05 | 0.33 | 0.44 | 0.26 |
| SW1295 | 1.00 | 0.80 | 0.55 | 4.39 | 0.73 | 0.50 | 0.70 | 0.35 |
| BAF57 | 1.00 | 0.98 | 0.93 | 0.54 | 1.82 | 0.54 | 0.70 | 0.92 |
| EAT/MCL-1 | 1.00 | 1.99 | 0.79 | 2.55 | 2.67 | 1.79 | 1.42 | 1.12 |
| Phospholipase Inhibitor | 1.00 | 0.19 | 1.35 | 0.23 | 6.03 | 0.11 | 0.59 | 0.23 |
| TSC-22 | 1.00 | 0.60 | 0.56 | 2.31 | 1.23 | 0.42 | 0.42 | 0.55 |
| Split hand/foot deleted 1 | 1.00 | 1.89 | 0.98 | 10.53 | 1.48 | 0.94 | 0.83 | 0.55 |
| Gamma Sarcoglycan | 1.00 | 0.47 | 1.51 | 0.46 | 0.43 | 0.48 | 0.44 | 0.14 |
| Nicotinamid | 1.00 | 0.93 | 1.00 | 2.15 | 1.14 | 0.86 | 0.92 | 0.78 |
| N-Methyltransferase | | | | | | | | |
| Golgi 4-Transmembrane | 1.00 | 0.57 | 0.28 | 2.00 | 0.53 | 0.26 | 0.27 | 0.11 |
| spanning transporter | | | | | | | | |
| UBC9 | 1.00 | 0.95 | 1.06 | 1.91 | 1.76 | 0.94 | 1.40 | 0.64 |
| Cathepsin C | 1.00 | 1.68 | 1.58 | 1.09 | 2.20 | 1.61 | 0.75 | 0.89 |
| tsg101 | 1.00 | 0.94 | 1.03 | 2.06 | 1.80 | 0.92 | 0.96 | 0.48 |
| DAD-1 | 1.00 | 1.07 | 0.93 | 1.93 | 1.32 | 1.04 | 0.87 | 0.61 |
| HMG-14 | 1.00 | 0.61 | 0.43 | 2.11 | 1.03 | 0.40 | 0.50 | 0.20 |
| TAK1 | 1.00 | 0.64 | 0.90 | 0.46 | 1.75 | 0.74 | 0.93 | 0.74 |
| IL-5Ralpha | 1.00 | 5.89 | 0.84 | 1.12 | 1.38 | 1.66 | 2.61 | 1.74 |
| Fer | 1.00 | 0.72 | 0.46 | 0.33 | 0.48 | 0.22 | 0.40 | 0.37 |

TABLE 2

| | Intact | | Intact | |
|---|---|---|---|---|
| | skin | Wound | skin | Wound |
| Genes differentially | control | control | diabetic | diabetic |
| expressed | animals | animals | mice | mice |
| TTF-1 | 1.00 | 0.61 | 1.57 | 0.54 |
| CCR-1 | 1.00 | 23.51 | 2.26 | 20.66 |
| MASPIN | 1.00 | 0.30 | 1.54 | 0.38 |
| B-Raf | 1.00 | 1.25 | 1.66 | 1.05 |
| Prothymosin alpha | 1.00 | 0.58 | 1.26 | 0.83 |
| Eps8 | 1.00 | 2.50 | 0.70 | 1.40 |
| KIAA1247 | 1.00 | 0.30 | 1.50 | 0.50 |
| Cystatin C | 1.00 | 0.49 | 1.35 | 0.57 |
| SW1136 | 1.00 | 0.31 | 1.15 | 0.48 |
| SW1295 | 1.00 | 0.39 | 1.00 | 0.65 |
| BAF57 | 1.00 | 0.45 | 1.15 | 0.51 |
| EAT/MCL-1 | 1.00 | 1.36 | 1.54 | 1.56 |
| Phospholipase Inhibitor | 1.00 | 0.31 | 1.14 | 0.28 |
| TSC-22 | 1.00 | 0.43 | 1.70 | 0.60 |
| Split hand/foot deleted 1 | 1.00 | 0.41 | 0.35 | 0.79 |
| Gamma Sarcoglycan | 1.00 | 0.04 | 0.33 | 0.08 |
| Nicotinamid N-Methyltransferase | 1.00 | 0.77 | 3.26 | 1.88 |
| Golgi 4-Transmembrane spanning | 1.00 | 0.22 | 0.35 | 0.40 |
| transporter | | | | |
| UBC9 | 1.00 | 0.63 | 1.66 | 0.63 |
| Cathepsin C | 1.00 | 0.89 | 0.58 | 0.34 |
| tsg101 | 1.00 | 0.64 | 1.53 | 0.53 |
| DAD-1 | 1.00 | 0.96 | 1.84 | 1.43 |
| HMG-14 | 1.00 | 0.31 | 1.56 | 0.58 |
| TAK1 | 1.00 | 0.35 | 1.30 | 0.46 |
| Fer | 1.00 | 0.27 | 1.35 | 0.31 |

TABLE 3

| | | PROTEIN- | | PROTEIN- | | | |
|---|---|---|---|---|---|---|---|
| No. | NAME | MOUSE* | Seq ID No. | HUMAN* | Seq ID No. | CDNA-MOUSE* | CDNA-HUMAN* |
| 1. | SW1136 | Seq ID Nr. 55 | 55 | Seq ID Nr. 56 | 56 | Seq ID Nr. 50 | Seq ID Nr. 51 |
| 2. | SW1295 | Seq ID Nr. 57 | 57 | trembl:Q9Y6H1 | 58 | Seq ID Nr. 52 | EMBL:AF078845 |

*pir:PIR-databank
EMBL:EMBL-databank
trembl:translated EMBL-databank

TABLE 4

| | | PROTEIN- | | PROTEIN- | | | |
|---|---|---|---|---|---|---|---|
| No. | NAME | MOUSE* | Seq ID No. | HUMAN* | Seq ID No. | CDNA-MOUSE* | CDNA-HUMAN* |
| 3. | tumor susceptibility gene 101 | SP:Q61187 | 1 | SP:Q99816 | 2 | EMBL:U52945 | EMBL:U82130 |
| | (TSG101) | | | | | | |
| 4. | MASPIN | SP:P70124 | 3 | SP:P36952 | 4 | EMBL:U54705 | EMBL:U04313 |
| 5. | Transcription Termination Factor 1 | SP:Q62187 | 5 | SP:Q15361 | 6 | EMBL:X83974 | EMBL:X83973 |
| 6. | B-Raf | SP:P28028 | 7 | SP:P15056 | 8 | GB:M64429 | GB:M95712 |
| 7. | Prothymosin alpha | SP:P26350 | 9 | SP:P06454 | 10 | GB:X56135 | GB:M14630 |
| 8. | Golgi 4-transmembrane spanning | SP:Q60961 | 11 | SP:Q15012 | 12 | EMBL:U34259 | EMBL:D14696 |
| | transporter (MTP) | | | | | | |
| 9. | CCR-1 | SP:P51675 | 13 | SP:P32246 | 14 | EMBL:U29678 | EMBL:L09230 |
| 10. | HMG-14 | SP:P18608 | 15 | SP:P05114 | 16 | EMBL:X53476 | EMBL:J02621 |
| 11. | Split hand/Foot deleted 1 | GP:NP_033195 | 17 | SP:Q13437 | 18 | EMBL:U41626 | EMBL:U41515 |
| 12. | TAK1 | SP:P49117 | 19 | SP:P49116 | 20 | EMBL:U11688 | EMBL:U10990 |
| 13. | BAF57 | trembl:O54941 | 31 | trembl:O43539 | 32 | GB:AF035263 | GB:AF035262 |
| 14. | EPS8 | SP:Q08509 | 33 | SP:Q12929 | 34 | EMBL:L21671 | EMBL:U12535 |
| 15. | KIAA1247 | Seq ID Nr. 36 | 35 | GP:BAA86561 | 36 | Seq ID 53 | GB:AB033073 |
| 16. | Phospholipase Inhibitor | Seq ID Nr. 38 | 37 | US 5,948,626 | 38 | Seq ID 54 | US 5,948,626 |
| 17. | EAT/MCL-1 | trembl:P97287 | 39 | SP:Q07820 | 40 | EMBL:U35623 | EMBL:L08246 |
| 18. | TSC-22 | SP:Q00992 | 41 | SP:Q15714 | 42 | EMBL:X62940 | EMBL:U35048 |
| 19. | Gamma-Sarcoglycan | trembl:P82348 | 43 | trembl:Q13326 | 44 | EMBL:AB024922 | EMBL:U34976 |
| 20. | Cystatin C | SP:P21460 | 46 | SP:P01034 | 47 | EMBL:M59470 | EMBL:X05607 |
| 21. | Fer | trembl:P70451 | 63 | SP:P16591 | 64 | EMBL:U76762 | EMBL:J03358 |
| 22. | MRP-3 | SP:P27784 | 65 | SP:P55773 | 66 | EMBL:M58004 | EMBL:U85767 |
| 23. | NNMT | trembl:O55239 | 67 | SP:P40261 | 68 | EMBL:U86105 | EMBL:U08021 |
| 24. | UBC9 | SP:P50550 | 69 | SP:P50550 | 70 | EMBL:X99739 | EMBL:X96427 |

*pir:PIR-databank
EMBL:EMBL-databank
trembl:translated EMBL-databank
GB:GeneBank nucleic acids
GP:GeneBank polypeptide

TABLE 5

| | | PROTEIN- | | PROTEIN- | | CDNA- | CDNA- |
|---|---|---|---|---|---|---|---|
| No. | NAME | MOUSE* | Seq ID No. | HUMAN* | Seq ID No. | MOUSE* | HUMAN* |
| 25. | MCP-3 | SP:Q03366 | 21 | SP:P80098 | 22 | EMBL:X70058 | EMBL:X71087 |
| 26. | IL-5Ralpha | SP:P21183 | 23 | SP:Q01344 | 24 | EMBL:D90205 | EMBL:M96652 |
| 27. | DAD-1 | SP:P46966 | 25 | SP:P46966 | 26 | EMBL:U22107 | EMBL:D15057 |
| 29. | MCP-2 | trembl:Q9Z121 | 29 | SP:P80075 | 30 | GB: | GB:X99886 |
| | | | | | | AB023418 | |
| 30. | Cathepsin C | SP:P97821 | 72 | SP:P53634 | 73 | GB:U89269 | GB:X87212 |

*pir:PIR-databank
EMBL:EMBL-databank
GB:GeneBank nucleic acid
GP:GeneBank polypeptide
trembl:translated EMBL-databank

TABLE 6

| Differentially | | | | | | | | | |
|---|---|---|---|---|---|---|---|---|---|
| expressed | Intakt | Wound | Wound | Wound | Wound | Wound | Wound | Wound | Wound |
| genes | skin | 1 h | 7 h | 15 h | 24 h | 3 d | 5 d | 7 d | 14 d |
| B-Raf | 1,00 | 1,30 | 0,79 | 0,89 | 0,87 | 0,94 | 1,21 | 0,72 | 0,93 |
| KIAA1247 | 1,00 | 0,67 | 0,53 | 0,30 | 0,31 | 0,44 | 0,67 | 1,11 | 0,97 |
| Cystatin C | 1,00 | 0,78 | 0,69 | 0,69 | 0,60 | 0,68 | 0,88 | 1,05 | 0,89 |
| SW1136 | 1,00 | 0,48 | 0,36 | 0,15 | 0,15 | 0,23 | 0,31 | 0,33 | 0,20 |
| SW1295 | 1,00 | 0,43 | 0,33 | 0,28 | 0,22 | 0,27 | 0,35 | 0,66 | 0,44 |
| BAF57 | 1,00 | 0,76 | 0,57 | 0,54 | 0,42 | 0,67 | 0,93 | 1,19 | 0,94 |
| EAT/MCL-1 | 1,00 | 0,58 | 0,81 | 1,08 | 0,75 | 0,85 | 1,16 | 0,66 | 0,66 |
| Phospholipase | 1,00 | 14,47 | 1,00 | 0,23 | 0,88 | 1,34 | 1,49 | 3,67 | 21,93 |
| Inhibitor | | | | | | | | | |
| TSC-22 | 1,00 | 0,68 | 0,66 | 0,43 | 0,40 | 0,43 | 0,70 | 0,88 | 0,95 |
| Split hand/foot | 1,00 | 0,57 | 0,40 | 0,42 | 0,43 | 0,53 | 0,67 | 0,62 | 0,66 |
| deleted 1 | | | | | | | | | |
| Nicotinamide | 1,00 | 0,36 | 0,60 | 0,46 | 0,43 | 0,71 | 0,43 | 0,80 | 0,62 |
| N-Methyl- | | | | | | | | | |
| transferase | | | | | | | | | |
| Golgi 4- | 1,00 | 0,67 | 0,69 | 0,34 | 0,26 | 0,33 | 0,46 | 0,50 | 0,48 |
| Transmem- | | | | | | | | | |
| brane span- | | | | | | | | | |
| ning trans- | | | | | | | | | |
| porter | | | | | | | | | |
| UBC9 | 1,00 | 0,72 | 0,68 | 0,49 | 0,51 | 0,81 | 0,87 | 0,95 | 1,18 |
| Cathepsin C | 1,00 | 0,83 | 0,29 | 0,26 | 1,54 | 0,81 | 1,29 | 1,07 | 3,88 |
| tsg101 | 1,00 | 0,71 | 0,51 | 0,45 | 0,33 | 0,62 | 0,53 | 0,66 | 0,83 |
| DAD-1 | 1,00 | 0,84 | 0,76 | 0,74 | 0,84 | 1,07 | 1,10 | 1,36 | 1,18 |
| HMG-14 | 1,00 | 0,55 | 0,31 | 0,29 | 0,33 | 0,46 | 0,49 | 0,45 | 0,62 |
| TAK1 | 1,00 | 0,49 | 0,62 | 0,28 | 0,39 | 0,54 | 0,64 | 0,71 | 0,65 |
| Fer | 1,00 | 0,17 | 0,15 | 0,09 | 0,13 | 0,17 | 0,22 | 0,25 | 0,08 |
| EPS8 | 1,00 | 0,60 | 1,10 | 1,00 | 0,70 | 0,70 | 1,00 | 0,70 | 0,90 |
| CCR-1 | 1,00 | 1,40 | 5,90 | 19,00 | 17,00 | 16,00 | 13,00 | 5,50 | 3,70 |
| MASPIN | 1,00 | 0,60 | 0,50 | 0,30 | 0,30 | 0,30 | 0,40 | 0,50 | 0,80 |
| TTF-1 | 1,00 | 0,80 | 0,60 | 0,50 | 0,40 | 0,50 | 0,60 | 0,60 | 0,90 |

TABLE 7

| cDNA expression | | | | | |
|---|---|---|---|---|---|
| in human biop- | Time after wounding | | | | |
| sies relative to | Intact | | | | |
| Cyclophilin | skin | wound 1 h | wound 24 h | wound 5 d | wound 14 d |
| Eps8 | 1,00 | 1,41 | 0,74 | 1,3 | 1,29 |
| KIAA1247 | 1,00 | 0,92 | 1,22 | 1,02 | 1,58 |
| Phospholipase | 1,00 | 1,24 | 0,64 | 0,57 | 0,85 |
| Inhibitor | | | | | |
| Cystatin C | 1,00 | 0,86 | 1,27 | 1,79 | 2,33 |
| TSC-22 | 1,00 | 1,34 | 0,91 | 1,29 | 1,98 |
| Golgi 4- | 1,00 | 0,69 | 0,63 | 0,59 | 0,72 |
| Transmem- | | | | | |
| brane spanning | | | | | |
| transporter | | | | | |
| Cathepsin C | 1,00 | 0,86 | 2,96 | 4,09 | 2,67 |
| HMG-14 | 1,00 | 1,87 | 0,56 | 0,98 | 0,87 |
| Nicotinamide | 1,00 | 1,74 | 6,20 | 9,74 | 5,15 |
| N- | | | | | |
| Methyltrans- | | | | | |
| ferase | | | | | |
| UBC9 | 1,00 | 0,98 | 1,39 | 1,43 | 1,45 |
| CCR-1 | 1,00 | 1,28 | 10,56 | 7,78 | 5,66 |
| tsg101 | 1,00 | 1,09 | 1,07 | 1,24 | 1,27 |
| MASPIN | 1,00 | 1,62 | 0,38 | 1,11 | 0,61 |
| TTF-1 | 1,00 | 0,55 | 0,95 | 0,74 | 1,45 |
| B-Raf | 1,00 | 1,82 | 0,89 | 1,45 | 1,30 |
| DAD-1 | 1,00 | 0,66 | 1,47 | 1,61 | 1,46 |
| Fer | 1,00 | 8,07 | 0,83 | 3,35 | 5,06 |
| Split hand/foot | 1,00 | 0,98 | 0,79 | 1,31 | 1,20 |
| deleted 1 | | | | | |
| Gamma | 1,00 | 1,13 | 0,29 | 0,67 | 0,89 |
| Sarcoglycan | | | | | |
| TAK1 | 1,00 | 1,08 | 0,89 | 1,37 | 1,56 |

TABLE 8

| Gen useable according | Intact skin of | wound edge of | wound ground of |
|---|---|---|---|
| to the invention | ulcer patients | ulcer patients | ulcer patients |
| Eps8 | 1.00 | 0.88 | 0.89 |
| K1AA1247 | 1.00 | 0.38 | 1.1 |
| Phospholipase In- | 1.00 | 0.54 | 0.48 |
| hibitor (Variant | | | |
| SEQ ID Nr. 45) | | | |
| Phospholipase In- | 1.00 | 1.54 | 1.18 |
| hibitor (Variant | | | |
| SEQ ID Nr. 81) | | | |
| Cystatin C | 1.00 | 0.66 | 0.52 |
| TSC-22 | 1.00 | 0.64 | 0.70 |
| Golgi 4- | 1.00 | 1.15 | 0.85 |
| Transmembrane | | | |
| spanning transporter | | | |
| Cathepsin C | 1.00 | 0.6 | 1.07 |
| HMG-14 | 1.00 | 1.64 | 0.87 |
| Nicotinamid N- | 1.00 | 2.28 | 1.49 |
| Methyltransferase | | | |
| UBC9 | 1.00 | 0.72 | 0.74 |
| CCR-1 | 1.00 | 6.00 | 16.80 |
| tsg101 | 1.00 | 0.54 | 0.50 |
| MASPIN | 1.00 | 0.29 | 0.04 |
| TTF-1 | 1.00 | 0.40 | 0.60 |
| B-Raf | 1.00 | 0.76 | 0.31 |
| DAD-1 | 1.00 | 0.97 | 0.65 |
| Fer | 1.00 | 0.80 | 0.26 |
| Split hand/foot | 1.00 | 0.19 | 0.16 |
| deleted 1 | | | |
| Gamma | 1.00 | 0.10 | 0.05 |
| Sarcoglycan | | | |
| TAK1 | 1.00 | 0.27 | 0.41 |

TABLE 9

| Gen useable | standardized relative amount | | |
|---|---|---|---|
| according to | Intact healthy | Lesional skin of | Non-lesional skin of |
| the invention | skin | psoriasis patients | psoriasis patients |
| KIAA1247 | 9.69E-02 | 6.23E-02 | 1.39E-01 |
| Eps8 | 1.10E-01 | 5.36E-02 | 1.41E-01 |
| MASPIN | 7.55E-02 | 8.60E-02 | 1.26E-01 |
| | | 1.26E-01 | |

What is claimed is:

1. A method of diagnosing a skin disorder in a patient, said method comprising determining, in a sample from said patient, the level of at least one polypeptide having an amino acid sequence selected from the group consisting of SEQ ID NOS: 1-20, 31-48, 55-58, 63-70, and 80-82, and/or a nucleic acid encoding one of said polypeptides, and/or an antibody specific for at least one of said polypeptides, a level of said polypeptide, said nucleic acid, or said antibody in said sample from said patient that differs from the level in a control sample indicating that said patient has a skin disorder.

2. A method of diagnosing a wound healing disorder in a patient, said method comprising determining, in a sample from said patient, the level of at least one polypeptide having an amino acid sequence selected from the group consisting of SEQ ID NOS: 1-26, 29-48, 55-58, 63-73, and 80-82, and/or a nucleic acid encoding one of said polypeptides, and/or an antibody specific for at least one of said polypeptides, a level of said polypeptide, said nucleic acid, or said antibody in said sample from said patient that differs from the level in a control sample indicating that said patient has a wound healing disorder.

3. A method of promoting wound healing or treating a wound healing disorder in a patient, said method comprising administering to a patient a therapeutically-effective amount of a polypeptide having an amino acid sequence selected from the group consisting of SEQ ID NOS: 1-26, 29-48, 55-58, 63-73, and 80-82, a nucleic acid encoding at least one of said polypeptides, an antibody specific for at least one of said polypeptides, and/or a cell expressing at least one of said polypeptides, optionally combined with suitable additives and/or auxiliaries.

4. A method of preventing or treating a skin disorder in a patient, said method comprising administering to a patient a therapeutically-effective amount of a polypeptide having an amino acid sequence selected from the group consisting of SEQ ID NOS: 1-20, 31-48, 55-58, 63-70, and 80-82, a nucleic acid encoding at least one of said polypeptides, an antibody specific for at least one of said polypeptides, and/or a cell expressing at least one of said polypeptides, optionally combined with suitable additives or auxiliaries.

5. A method of identifying a candidate pharmacologically active substance for a skin disorder, said method comprising contacting a polypeptide having an amino acid sequence selected from the group consisting of SEQ ID NOS: 1-20, 31-48, 55-58, 63-70, and 80-82 with a candidate substance, and determining whether said candidate substance interacts with said polypeptide, the ability of said candidate substance to interact with said polypeptide identifying a candidate pharmacologically active substance for a skin disorder.

6. A method of identifying a candidate pharmacologically active substance for a wound healing disorder, said method comprising contacting a polypeptide having an amino acid sequence selected from the group consisting of SEQ ID NOS: 1-26, 29-48, 55-58, 63-73, and 80-82 with a candidate substance, and determining whether said candidate substance interacts with said polypeptide, the ability of said candidate substance to interact with said polypeptide identifying a candidate pharmacologically active substance for a wound healing disorder.

7. A method of identifying a candidate pharmacologically active substance for a skin disorder, said method comprising contacting a cell exhibiting pathologically disturbed expression of a polypeptide having an amino acid sequence selected from the group consisting of SEQ ID NOS: 1-20, 31-48, 55-58, 63-70, and 80-82, or a nucleic acid encoding one of said polypeptides, with a candidate substance, and determining whether said polypeptide or nucleic acid encoding said polypeptide returns to normal expression, the ability to mediate a return to normal expression indicating that said candidate substance is a candidate pharmacologically active substance for a skin disorder.

8. A method of identifying a candidate pharmacologically active substance for a wound healing disorder, said method comprising contacting a cell exhibiting pathologically disturbed expression of a polypeptide having an amino acid sequence selected from the group consisting of SEQ ID NOS: 1-26, 29-48, 55-58, 63-73, and 80-82, or a nucleic acid encoding one of said polypeptides, with a candidate substance, and determining whether said polypeptide or nucleic acid encoding said polypeptide returns to normal expression, the ability to mediate a return to normal expression indicating that said candidate substance is a candidate pharmacologically active substance for a wound healing disorder.

9. The method of claim 1 or 2, wherein said sample is a skin cell sample.

10. The method of claim 7 or 8, wherein said cell is a skin cell.

11. The method of claim 1 or 2, wherein the level of said polypeptide is determined using an antibody specific for at least one of SEQ ID NOS: 1-26, 29-48, 55-58, 63-73, and 80-82.

12. An array of polypeptides having the amino acid sequences of SEQ ID NO: 55-58, nucleic acids encoding said polypeptides, antibodies specific for said polypeptides, or cells expressing said polypeptides attached to a carrier material.

13. The array of claim 12, wherein said array further comprises one or more polypeptides having the amino acid sequences selected from the group consisting of SEQ ID NOS: 1-26, 29-48, 63-73, and 80-82, one or more nucleic acids encoding said polypeptides, one or more antibodies specific for said polypeptides, or cells expressing one or more of said polypeptides.

14. An isolated and purified polypeptide having the amino acid sequence of SEQ ID NO: 55, SEQ ID NO: 56, SEQ ID NO: 57, or SEQ ID NO: 58, or a nucleic acid encoding said polypeptides.

15. A method of promoting wound healing, treating a wound healing disorder, or preventing or treating a skin disorder in a patient, said method comprising administering to said patient a pharmacologically active substance identified by the method of any one of claims 5-8.

16. The method of claim 3 or 4, wherein the polypeptide is Eps8.

17. The method of claim 3 or 4, wherein the nucleic acid is selected from the group consisting of SEQ ID NOS: 85 and 86.

18. The method of claim 3 or 4, wherein the nucleic acid is inserted into a vector.

19. The method of claim 18, wherein the vector is an expression vector containing an expression construct comprising in 5′ to 3′ direction
(i) a promoter for the control of the transcription of the nucleic acid;
(ii) the nucleic acid, and
(iii) a polyA sequence.

20. The method of claim 18, wherein the vector is a viral vector, the virus is selected from the group consisting of baculoviruses, vaccinia viruses, adenoviruses, adeno-associated viruses, retroviruses and herpesviruses.

21. The method of claim 18, wherein the vector is a non-viral vector selected from the group consisting of a virosome, a liposome, a naked DNA, and a polylysine-conjugated DNA.

22. The method of claim 3 or 4, wherein the polypeptide, the nucleic acid, the antibody and/or the cell is formulated in a pharmaceutically acceptable carrier.

23. The method of claim 3 or 4, wherein the polypeptide, the nucleic acid, the antibody and/or the cell is administered by a method selected from the group consisting of topical administration, systemic administration, local injection, intradermal administration and subcutaneous administration.

24. The method of claim 3, wherein the wound healing disorder is selected from the group consisting of a wound infected with an organism, a wound infected with a virus, an ischemic wound and a wound from a patient suffering from a condition selected from the group consisting of diabetes, alcoholism, arterial disorder and venous insufficiency.

25. The method of claim 24, wherein the wound is a wound from a patient suffering from diabetes.

26. The method of claim 24, wherein the wound is a badly healing wound selected from the group consisting of a diabetic wound, a diabetic ulcer, a neuropathic wound, a venous ulcer, and an arterial ulcer.

27. The method of claim 4, wherein the skin disorder is a disorder selected from the group consisting of psoriasis, eczema, acne, urticaria, defective skin pigmentation, defective hair growth, defective hair metabolism and senile skin.


SEQUENCE LISTING (86 sequences)

SEQ ID NO: 1 - protein, 391 aa, Mus musculus
MAVSESQLKKMMSKYKYRDLTVRQTVNVIAMYKDLKPVLDSYVFNDGSSRELVNLTGTIPVRYRGNIYNIPICLWLLDTYPYNPPICFVKPTSSMTIKTGKHVDANGKIYLPYLHDWKHPRSELLELIQIMIVIFGEEPPVFSRPTVSASYPPYTATGPPNTSYMPGMPSGISAYPSGYPPNPSGYPGCPYPPAGPYPATTSSQYPSQPPVTTVGPSRDGTISEDTIRASLISAVSDKLRWRMKEEMDGAQAELNALKRTEEDLKKGHQKLEEMVTRLDQEVAEVDKNIELLKKKDEELSSALEKMENQSENNDIDEVIIPTAPLYKQILNLYAEENAIEDTIFYLGEALRRGVIDLDVFLKHVRLLSRKQFQLRALMQKARKTAGLSDLY

SEQ ID NO: 2 - protein, 390 aa, Mus musculus
MAVSESQLKKMVSKYKYRDLTVRETVNVITLYKDLKPVLDSYVFNDGSSRELMNLTGTIPVPYRGNTYNIPICLWLLDTYPYNPPICFVKPTSSMTIKTGKHVDANGKIYLPYLHEWKHPQSDLLGLIQVMIVVFGDEPPVFSRPISASYPPYQATGPPNTSYMPGMPGGISPYPSGYPPNPSGYPGCPYPPGGPYPATTSSQYPSQPPVTTVGPSRDGTISEDTIRASLISAVSDKLRWRMKEEMDRAQAELNALKRTEEDLKKGHQKLEEMVTRLDQEVAEVDKNIELLKKKDEELSSALEKMENQSENNDIDEVIIPTAPLYKQILNLYAEENAIEDTIFYLGEALRRGVIDLDVFLKHVRLLSRKQFQLRALMQKARKTAGLSDLY

SEQ ID NO: 3 - protein, 375 aa, Mus musculus
MDALRLANSAFAVDLFKQLCERDPAGNILFSPICLSTSLSLAQVGTKGDTANEIGQVLHFENVKDVPFGFQTVTSDVNKLSSFYSLKLVKRLYIDKSLNPSTEFISSTKRPYAKELETVDFKDKLEETKGQINSSIKELTDGHFEDILSENSISDQTKILVVNAAYFVGKWMKKFPESETKECPFRISKTDTKPVQMMNLEATFCLGNIDDISCKIIELPFQNKHLSMLIVLPKDVEDESTGLEKIEQQLNPETLLQWTNPSTMANAKVKLSLPKFKVEKMIDPKASLESLGLKSLFNESTSDFSGMSETKGVSLSNVIHRVCLEITEDGGESIEVPGSRILQHKDEFNADHPFIYIIRHNKTRNIIFFGKFCSP

SEQ ID NO: 4 - protein, 375 aa, Homo sapiens
MDALQLANSAFAVDLFKQLCEKEPLGNVLFSPICLSTSLSLAQVGAKGDTANEIGQVLHFENVKDIPFGFQTVTSDVNKLSSFYSLKLIKRLYVDKSLNLSTEFISSTKRPYAKELETVDFKDKLEETKGQINNSIKDLTDGHFENILADNSVNDQTKILVVNAAYFVGKWMKKFPESETKECPFRLNKTDTKPVQMMNMEATFCMGNIDSINCKIIELPFQNKHLSMFILLPKDVEDESTGLEKIEKQLNSESLSQWTNPSTMANAKVKLSIPKFKVEKMIDPKACLENLGLKHIFSEDTSDFSGMSETKGVALSNVIHKVCLEITEDGGDSIEVPGARILQHKDELNADHPFIYIIRHNKTRNIIFFGKFCSP

SEQ ID NO: 5 - protein, 833 aa, Mus musculus
MKGGTSKFKTHTETLYKKKKWSSVSEKRPQKCPSQCLESKQPQVSVLGKRRRASQTPAQETLESEWPQKAKKKKRRGEPQTPTQESLESEQPPVSLLGKRRRESQTPAQENSESEQPRKAKRRRKKRKGSQQPTSSLLKTPETFLKAKKTTSAHKKKKNSVLEVDMETGIILVDKENMENLLETSRKDVDIVYVDMSKGQRSAKVRETGELPAAKPQEHGCRELLGDVRSRKKQKHLQKVAPWDVVQGSQPESISLPPSEPLSSEDLEGKSTEAAVFCKRSLKKNVFRSQELEPIPDSLDDSETISERLDSTHHGGAVGAGECESTKESHSIKKKSKKKKHKSVALATSSDSASVTDSKAKNALVDSSEGSGAVREEDVDHRPAEAEAQACSTEKHREAMQRLEPTHEEESNSESASNSAARHISEDRRESDDSDVDLGSAVRQLREFIPDIQERAATTIRRMYRDDLGLFKEFKAQGVAIRFGKFSAKENKQIEKNVQDFLSLTGIESADKLLYTDRYPEEKTLITNLKRKHAFRLHIGKGIARPWKLVYYRAKKIFDVNNYKGRYNEEDTKKLKAYHSLHGNDWKKIGAMVARSSLSVALKFSQIGGTRNQGAWSKAETQRLIKAVEDVILKKMSPQELRELDSKLQEDPEGRLSIVREKLYKGISWVEVEARVETRNWMQCKSKWTEILTKRMTHGGFVYRGVNALQAKITLIERLYELNVNDANEIDWEDLCSAIGDVPPPFVQAKFYKLKAACVPFWQKKTFPEIIDYLYKNSLPLLKEKLDKKMKKKDGQIQTPAAPKQDFLFKDIFHCDDDSDEGSPEEPSASDVQ

SEQ ID NO: 6 - protein, 886 aa, Homo sapiens [sequence omitted]

SEQ ID NO: 7 - protein, 659 aa, Mus musculus
MEGGCGEGGGGTGSGRSAAAARRAGRMRPRAQGPDSESGGEASRLNLLDTCAVCHQNIQSRVPKLLPCLHSFCQRCLPAPQRYLMLTAPALGSAETPPPAPAPAPAPGSPAGGPSPFATQVGVIRCPVCSQECAERHIIDNFFVKDTTEVPSSTVEKSNQVCTSCEDNAEANGFCVECVEWLCKTCIRAHQRVKFTKDHTVRQKEEVSPEAVGVTSQRPVFCPFHKKEQLKLYCETCDKLTCRDCQLLEHKEHRYQFIEEAFQNQKVIIDTLITKLMEKTKYIKYTGNQIQNRIIEINQNQKQVEQDIKVAIFTLMVEINKKGKALLHQLEKTLGRRDSSDDWEIPDGQITVGQRIGSGSFGTVYKGKWHGDVAVKMLNVTAPTPQQLQAFKNEVGVLRKTRHVNILLFMGYSTKPQLAIVTQWCEGSSLYHHLHIIETKFEMIKLIDIARQTAQGMDYLHAKSIIHRDLKSNNIFLHEDLTVKIGDFGLATVKSRWSGSHQFEQLSGSILWMAPEVIRMQDKNPYSFQSDVYAFGIVLYELMTGQLPYSNINNRDQIIFMVGRGYLSPDLSKVRSNCPKAMKRLMAECLKKKRDERPLFPQILASIELLARSLPKIHRSASEPSLNRAGFQTEDFSLYACASPKTPIQAGGYGGFPVH

SEQ ID NO: 8 - protein, 765 aa, Homo sapiens
MAALSGGGGGGAEPGQALFNGDMEPEAGAGRPAASSAADPAIPEEVWNIKQMIKLTQEHIEALLDKFGGEHNPPSIYLEAYEEYTSKLDALQQREQQLLESLGNGTDFSVSSSASMDTVTSSSSSSLSVLPSSLSVFQNPTDVARSNPKSPQKPIVRVFLPNKQRTVVPARCGVTVRDSLKKALMMRGLIPECCAVYRIQDGEKKPIGWDTDISWLTGEELHVEVLENVPLTTHNFVRKTFFTLAFCDFCRKLLFQGFRCQTCGYKFHQRCSTEVPLMCVNYDQLDLLFVSKFFEHHPIPQEEASLAETALTSGSSPSAPASDSIGPQILTSPSPSKSIPIPQPFRPADEDHRNQFGQRDRSSSAPNVHINTIEPVNIDDLIRDQGFRGDGGSTTGLSATPPASLPGSLTNVKALQKSPGPQRERKSSSSSEDRNRMKTLGRRDSSDDWEIPDGQITVGQRIGSGSFGTVYKGKWHGDVAVKMLNVTAPTPQQLQAFKNEVGVLRKTRHVNILLFMGYSTKPQLAIVTQWCEGSSLYHHLHIIETKFEMIKLIDIARQTAQGMDYLHAKSIIHRDLKSNNIFLHEDLTVKIGDFGLATVKSRWSGSHQFEQLSGSILWMAPEVIRMQDKNPYSFQSDVYAFGIVLYELMTGQLPYSNINNRDQIIFMVGRGYLSPDLSKVRSNCPKAMKRLMAECLKKKRDERPLFPQILASIELLARSLPKIHRSASEPSLNRAGFQTEDFSLYACASPKTPIQAGGYGAFPVH

SEQ ID NO: 9 - protein, 111 aa, Mus musculus
MSDAAVDTSSEITTKDLKEKKEVVEEAENGRDAPANGNAQNEENGEQEADNEVDEEEEEGGEEEEEEEEGDGEEEDGDEDEEAEAPTGKRVAEDDEDDDVDTKKQKTEEDD

SEQ ID NO: 10 - protein, 111 aa, Homo sapiens
MSDAAVDTSSEITTKDLKEKKEVVEEAENGRDAPANGNAENEENGEQEADNEVDEEEEEGGEEEEEEEEGDGEEEDGDEDEEAESATGKRAAEDDEDDDVDTKKQKTDEDD

SEQ ID NO: 11 - protein, 233 aa, Mus musculus [sequence omitted]

SEQ ID NO: 12 - protein, 233 aa, Hoimo sapiens
MVSMSFKRNRSDRFYSTRCCGCCHVRTGTIILGTWYMVVNLLMAILLTVEVTHPNSMPAVNIQYEVIGNYYSSERMADNACVLFAVSVLMFIISSMLVYGAISYQVGWLIPFFCYRLFDFVLSCLVAISSLTYLPRIKEYLDQLPDFPYKDDLLALDSSCLLFIVLVFFALFIIFKAYLINCVWNCYKYINNRNVPEIAVYPAFEAPPQYVLPTYEMAVKMPEKEPPPPYLPA

SEQ ID NO: 13 - protein, 355 aa, Mus musculus
MEISDFTEAYPTTTEFDYGDSTPCQKTAVRAFGAGLLPPLYSLVFIIGVVGNVLMILVLMQHRRLQSMTSIYLFNLAVSDLVFLFTLPFWIDYKLKDDWIFGDAMCKLLSGFYYLGLYSEIFFIILLTIDRYLAIVHAVFALRARTVTLGIITSIITWALAILASMPALYFFKAQWEFTHRTCSPHFPYKSLKQWKRFQALKLNLLGLILPLLVMIICYAGIIRILLRRPSEKKVKAVRLIFAITLLFFLLWTPYNLSVFVSAFQDVLFTNQCEQSKHLDLAMQVTEVIAYTHCCVNPIIYVFVGERFWKYLRQLFQRHVAIPLAKWLPFLSVDQLERTSSISPSTGEHELSAGF

SEQ ID NO: 14 - protein, 355 aa, Homo sapiens
METPNTTEDYDTTTEFDYGDATPCQKVNERAFGAQLLPPLYSLVFVIGLVGNILVVLVLVQYKRLKNMTSIYLLNLAISDLLFLFTLPFWIDYKLKDDWVFGDAMCKILSGFYYTGLYSEIFFIILLTIDRYLAIVHAVFALRARTVTFGVITSIIIWALAILASMPGLYFSKTQWEFTHHTCSLHFPHESLREWKLFQALKLNLFGLVLPLLVMIICYTGIIKILLRRPNEKKSKAVRLIFVIMIIFFLFWTPYNLTILISVFQDFLFTHECEQSRHLDLAVQVTEVIAYTHCCVNPVIYAFVGERFRKYLRQLFHRRVAVHLVKWLPFLSVDRLERVSSTSPSTGEHELSAGF

SEQ ID NO: 15 - protein, 96 aa, Mus musculus
MPKRKVSADGAAKAEPKRRSARLSAKPAPAKVDAKPKKAAGKDKASDKKVQIKGKRGAKGKQADVADQQTTELPAENGETENQSPASEEEKEAKSD

SEQ ID NO: 16 - protein, 100 aa, Homo sapiens
MPKRKVSSAEGAAKEEPKRRSARLSAKPPAKVEAKPKKAAAKDKSSDKKVQTKGKRGAKGKQAEVANQETKEDLPAENGETKTEESPASDEAGEKEAKSD

SEQ ID NO: 17 - protein, 70 aa, Mus musculus
MSEKKQPVDLGLLEEDDEFEEFPAEDWAGLDEDEDAHVWEDNWDDDNVEDDFSNQLRAELEKHGYKMETS

SEQ ID NO: 18 - protein, 70 aa, Homo sapiens; identical to SEQ ID NO: 17

SEQ ID NO: 19 - protein, 596 aa, Mus musculus
MTSPSPRIQIISTDSAVASPQRIQIVTDQQTGQKIQIVTAVDASGSSKQQFILTSPDGAGTGKVILASPETSSAKQLIFTTSDNLVPGRIQIVTDSASVERLLGKADVQRPQVVEYCVVCGDKASGRHYGAVSCEGCKGFFKRSVRKNLTYSCRSSQDCIINKHHRNRCQFCRLKKCLEMGMKMESVQSERKPFDVQREKPSNCAASTEKIYIRKDLRSPLIATPTFVADKDGARQTGLLDPGMLVNIQQPLIREDGTVLLAADSKAETSQGALGTLANVVTSLANLSESLNNGDASEMQPEDQSASEITRAFDTLAKALNTTDSASPPSLADGIDASGGGSIHVISRDQSTPIIEVEGPLLSDTHVTFKLTMPSPMPEYLNVHYICESASRLLFLSMHWARSIPAFQALGQDCNTSLVRACWNELFTLGLAQCAQVMSLSTILAAIVNHLQNSIQEDKLSGDRIKQVMEHIWKLQEFCNSMAKLDIDGYEYAYLKAIVLFSPDHPGLTGTSQIEKFQEKAQMELQDYVQKTYSEDTYRLARILVRLPALRLMSSNITEELFFTGLIGNVSIDSIIPYILKMETAEYNGQITGASL

SEQ ID NO: 20 - protein, 596 aa, Homo sapiens
MTSPSPRIQIISTDSAVASPQRIQIVTDQQTGQKIQIVTAVDASGSPKQQFILTSPDGAGTGKVILASPETSSAKQLIFTTSDNLVPGRIQIVTDSASVERLLGKTDVQRPQVVEYCVVCGDKASGRHYGAVSCEGCKGFFKRSVRKNLTYSCRSNQDCIINKHHRNRCQFCRLKKCLEMGMKMESVQSERKPFDVQREKPSNCAASTEKIYIRKDLRSPLIATPTFVADKDGARQTGLLDPGMLVNIQQPLIREDGTVLLATDSKAETSQGALGTLANVVTSLANLSESLNNGDTSEIQPEDQSASEITRAFDTLAKALNTTDSSSSPSLADGIDTSGGGSIHVISRDQSTPIIEVEGPLLSDTHVTFKLTMPSPMPEYLNVHYICESASRLLFLSMHWARSIPAFQALGQDCNTSLVRACWNELFTLGLAQCAQVMSLSTILAAIVNHLQNSIQEDKLSGDRIKQVMEHIWKLQEFCNSMAKLDIDGYEYAYLKAIVLFSPDHPGLTSTSQIEKFQEKAQMELQDYVQKTYSEDTYRLARILVRLPALRLMSSNITEELFFTGLIGNVSIDSIIPYILKMETAEYNGQITGASL

SEQ ID NO: 21 - protein, 97 aa, Mus musculus
MRISATLLCLLLIAAAFSIQVWAQPDGPNASTCCYVKKQKIPKRNLKSYRRITSSRCPWEAVIFKTKKGMEVCAEAHQKWVEEAIAYLDMKTPTPKP

SEQ ID NO: 22 - protein, 99 aa, Homo sapiens
MKASAALLCLLLTAAAFSPQGLAQPVGINTSTTCCYRFINKKIPKQRLESYRRTTSSHCPREAVIFKTKLDKEICADPTQKWVQDFMKHLDKKTQTPKL

SEQ ID NO: 23 - protein, 415 aa, Mus musculus
MVPVLLILVGALATLQADLLNHKKFLLLPPVNFTIKATGLAQVLLHWDPNPDQEQRHVDLEYHVKINAPQEDEYDTRKTESKCVTPLHEGFAASVRTILKSSHTTLASSWVSAELKAPPGSPGTSVTNLTCTTHTVVSSHTHLRPYQVSLRCTWLVGKDAPEDTQYFLYYRFGVLTEKCQEYSRDALNRNTACWFPRTFINSKGFEQLAVHINGSSKRAAIKPFDQLFSPLAIDQVNPPRNVTVEIESNSLYIQWEKPLSAFPDHCFNYELKIYNTKNGHIQKEKLIANKFISKIDDVSTYSIQVRAAVSSPCRMPGRWGEWSQPIYVGKERKSLVEWHLIVLPTAACFVLLIFSLICRVCHLWTRLFPPVPAPKSNIKDLPVVTEYEKPSNETKIEVVHCVEEVGFEVMGNSTF

SEQ ID NO: 24 - protein, 420 aa, Homo sapiens [sequence omitted]

SEQ ID NO: 25 - protein, 113 aa, Mus musculus
MSASVVSVISRFLEEYLSSTPQRLKLLDGYLLYILLTGALQFGYCLLVGTFPFNSFLSGFISCVGSFILAVCLRIQINPQNKADFQGISPERAFADFLFGSTILHLVVMNFVG

SEQ ID NO: 26 - protein, 113 aa, Homo sapiens
MSASVVSVISRFLEEYLSSTPQRLKLLDAYLLYILLTGALQFGYCLLVGTFPFNSFLSGFISCVGSFILAVCLRIQINPQNKADFQGISPERAFADFLFASTILHLVVMNFVG

SEQ ID NO: 27-28 - skipped (2 entries)

SEQ ID NO: 29 - protein, 97 aa, Mus musculus
MKIYAVLLCLLLIAVPVSPEKLTGPDKAPVTCCFHVLKLKIPLRVLKSYERINNIQCPMEAVVFQTKQGMSLCVDPTQKWVSEYMEILDQKSQILQP

SEQ ID NO: 30 - protein, 99 aa, Homo sapiens
MKVSAALLCLLLMAATFSPQGLAQPDSVSIPITCCFNVINRKIPIQRLESYTRITNIQCPKEAVIFKTQRGKEVCADPKERWVRDSMKHLDQIFQNLKP

SEQ ID NO: 31 - protein, 411 aa, Mus musculus
MSKRPSYAPPPTPAPATQMPSTPGFVGYNPYSHLAYNNYRLGGNPGTNSRVTASSGITIPKPPKPPDKPLMPYMRYSRKVWDQVKASNPDLKLWEIGKIIGGMWRDLTDEEKQEYLNEYEAEKIEYNESMKAYHNSPAYLAYINAKSRAEAALEEESRQRQSRMEKGEPYMSIQPAEDPDDYDDGFSMKHTATARFQRNHRLISEILSESVVPDVRSVVTTARMQVLKRQVQSLMVHQRKLEAELLQIEERHQEKKRKFLESTDSFNNELKRLCGLKVEVDMEKIAAEIAQAEEQARKRQEEREKEAAEQAERSQSSMAPEEEQVANKAEEKKDEESIPMETEETHLEDTAESQQNGEEGTSTPEDKESGQEGVDSMEVEGTSDSNTGSESNSATVEEPPTDPVPEDEKKE

SEQ ID NO: 32 - protein, 411 aa, Homo sapiens
MSKRPSYAPPPTPAPATQMPSTPGFVGYNPYSHLAYNNYRLGGNPGTNSRVTASSGITIPKPPKPPDKPLMPYMRYSRKVWDQVKASNPDLKLWEIGKIIGGMWRDLTDEEKQEYLNEYEAEKIEYNESMKAYHNSPAYLAYINAKSRAEAALEEESRQRQSRMEKGEPYMSIQPAEDPDDYDDGFSMKHTATARFQRNHRLISEILSESVVPDVRSVVTTARMQVLKRQVQSLMVHQRKLEAELLQIEERHQEKKRKFLESTDSFNNELKRLCGLKVEVDMEKIAAEIAQAEEQARKRQEEREKEAAEQAERSQSSIVPEEEQAANKGEEKKDDENIPMETEETHLEETTESQQNGEEGTSTPEDKESGQEGVDSMAEEGTSDSNTGSESNSATVEEPPTDPIPEDEKKE

SEQ ID NO: 33 - protein, 821 aa, Mus musculus
MNGHMSNRSSGYGVYPSQLNGYGSSPPYSQMDREHSSRTSAKALYEQRKNYARDSVSSVSDVSQYRVEHLTTFVLDRKDAMITVEDGIRKLKLLDAKGKVWTQDMILQVDDRAVSLIDLESKNELENFPLNTISHCQAVVHACSYDSILALVCKEPTQSKPDLHLFQCDEVKANLISEDIESAISDSKGGKQKRRPEALRMIAKADPGIPPPPRAPAPVPPGTVTQVDVRSRVAAWSAWAADQGDFEKPRQYHEQEETPEMMAARIDRDVQILNHILDDIEFFITKLQKAAEAFSELSKRKKSKKSKRKGPGEGVLTLRAKPPPPDEFVDCFQKFKHGFNLLAKLKSHIQNPSASDLVHFLFTPLNMVVQATGGPELASSVLSPLLTKDTVDFLNYTATAEERKLWMSLGDSWVKVRAEWPKEQFIPPYVPRFRNGWEPPMLNFMGAPTEQDMYQLAESVANAEHQRKQDSKRLSTEHSNVSDYPPADGYAYSSSMYHRGPHADHGEAAMPFKSTPNHQVDRNYDAVKTQPKKYAKSKYDFVARNSSELSVMKDDVLEILDDRRQWWKVRNASGDSGFVPNNILDIMRTPESGVGRADPPYTHTIQKQRTEYGLRSADTPSAPSPPPTPAPVPVPLPPSVPAPVSVPKVPADVTRQNSSSSDSGGSIVRDSQRYKQLPVDRRKSQMEEVQDELFQRLTIGRSAAQRKFHVPRQNVPVINITYDSSPEEVKTWLQSKGFNPVTVNSLGVLNGAQLFSLNKDELRSVCPEGARVFNQITVQKAALEDSNGSSELQEIMRRRQEKISAAASDSGVESFDEGSSH

SEQ ID NO: 34 - protein, 822 aa, Homo sapiens
MNGHISNHPSSFGMYPSQMNGYGSSPTFSQTDREHGSKTSAKALYEQRKNYARDSVSSVSDISQYRVEHLTTFVLDRKDAMITVDDGIRKLKLLDAKGKVWTQDMILQVDDRAVSLIDLESKNELENFPLNTIQHCQAVMHSCSYDSVLALVCKEPTQNKPDLHLFQCDEVKANLISEDIESAISDSKGGKQKRRPDALRMISNADPSIPPPPRAPAPAPPGTVTQVDVRSRVAAWSAWAADQGDFEKPRQYHEQEETPEMMAARIDRDVQILNHILDDIEFFITKLQKAAEAFSELSKRKKNKKGKRKGPGEGVLTLRAKPPPPDEFLDCFQKFKHGFNLLAKLKSHIQNPSAADLVHFLFTPLNMVVQATGGPELASSVLSPLLNKDTIDFLNYTVNGDERQLWMSLGGTWMKARAEWPKEQFIPPYVPRFRNGWEPPMLNFMGATMEQDLYQLAESVANVAEHQRKQEIKRLSTEHSSVSEYHPADGYAFSSNIYTRGSHLDQGEAAVAFKPTSNRHIDRNYEPLKTQPKKYAKSKYDFVARNNSELSVLKDDILEILDDRKQWWKVRNASGDSGFVPNNILDIVRPPESGLGRADPPYTHTIQKQRMEYGPRPADTPPAPSPPPTPAPVPVPLPPSTPAPVPVSKVPANITRQNSSSSDSGGSIVRDSQRHKQLPVDRRKSQMEEVQDELIHRLTIGRSAAQKKFHVPRQNVPVINITYDSTPEDVKTWLQSKGFNPVTVNSLGVLNGAQLFSLNKDELRTVCPEGARVYSQITVQKAALEDSSGSSELQEIMRRRQEKISAAASDSGVESFDEGSSH

SEQ ID NO: 35 - protein, 470 aa, Mus musculus
  VARIANT 371: Xaa = Any Amino Acid
MLVETGELDNTYILYTADHGYHIGQFGLVKGKSMPYEFDIRVPFYVRGPNVEAGSLNPHIVLNIDLGPHHTGYRWTGHPCRHGREVYSQTTGLRAASEPVPLEKEAEGLGETPSWWREANLLHKREGDKVNAQEENFLPKYQRVKDLCQRAEYQTACEQLGQKWQCVEDASGTLKLHKCKGPMRFGGGGGSRALSNLVPKYDGQSSEACSCDSGGGGDYKLGLAGRRKLFKKKYKTSYARNRSIRSVAIEVDGEIYHVGLDTVPQPRNLSKPHWPGAPEDQDDKDGGSFSGTGGLPDYSAPNPIKVTHRCYILENDTVQCDLDLYKSLQAWKDHKLHIDHEIETLQNKIKNLREVRGHLKKKRPEECDCHXISYHSQHKGRLKHKGSSLHPFRKGLQEKDKVWLLRDRNARRNCATAQTAAEQRYVQHARPHVLYPRQPPLADGATLDAGAVLRLHQRQQQHVLVLEDHK

SEQ ID NO: 36 - protein, 690 aa, Homo sapiens
EKHGSDYSKDYLTDLITNDSVSFFRTSKKMYPHRPVLMVISHAAPHGPEDSAPQYSRLFPNASQHITPSYNYAPNPDKHWIMRYTGPMKPIHMEFTNMLQRKRLQTLMSVDDSMETIYNMLVETGELDNTYIVYTADHGYHIGQFGLVKGKSMPYEFDIRVPFYVRGPNVEAGCLNPHIVLNIDLAPTILDIAGLDIPADMDGKSILKLLDTERPVNRFHLKKKMRVWRDSFLVERGKLLHKRDNDKVDAQEENFLPKYQRVKDLCQRAEYQTACEQLGQKWQCVEDATGKLKLHKCKGPMRLGGSRALSNLVPKYYGQGSEACTCDSGDYKLSLAGRRKKLFKKKYKASYVRSRSIRSVAIEVDGRVYHVGLGDAAQPRNLTKRHWPGAPEDQDDKDGGDFSGTGGLPDYSAANPIKVTHRCYILENDTVQCDLDLYKSLQAWKDHKLHIDHEIETLQNKIKNLREVRGHLKKKRPEECDCHKISYHTQHKGRLKHRGSSLHPFRKGLQEKDKVWLLREQKRKKKLRKLLKRLQNNDTCSMPGLTCFTHDNQHWQTAPFWTLGPFCACTSANNNTYWCMRTINETHNFLFCEFATGFLEYFDLNTDPYQLMNAVNTLDRDVLNQLHVQLMELRSCKGYKQCNPRTRNMDLGLKDGGSYEQYRQFQRRKWPEMKRPSSKSLGQLWEGWEG

SEQ ID NO: 37 - protein, 212 aa, Mus musculus
MILFRRHRTFLLAFTLLCTLLGLGCPLTCEVCKGSGHTCSGKMKTCEDGKDACVVLVSESSTKGRKSVNTFKACMKYKDCYSGFVSTTMTPSDYMVSNAHCCQSDGCNSGSVPPPLNNRTENGLMCPSCIAPFQETCPGTQAARCVGRETHCIYFAGNVQAGIIHTKFATRGCATESACHTKAGAEVPSAFYLYFLRRADCLPAPYPPGRGE

SEQ ID NO: 38 - protein, 204 aa, Homo sapiens
MRLSRRPETFLLAFVLLCTLLGLGCPLHCEICTAAGSRCHGQMKTCSSDKDTCVLLVGKATSKGKELVHTYKGCIRSQDCYSGVISTTMGPKDHMVTSSFCCQSDGCNSAFLSVPLTNLTENGLMCPACTASFRDKCMGPMTHCTGKENHCVSLSGHVQAGIFKPRFAMRGCATESMCFTKPGAEVPTGTNVLFLHHIECTHSP

SEQ ID NO: 39 - protein, 331 aa, Mus musculus
MFGLRRNAVIGLNLYCGGASLGAGGGSPAGARLVAEEAKARREGGGEAALLPGARVVARPPPVGAEDPDVTASAERRLHKSPGLLAVPPEEMAASAAAAIVSPEEELDGCEPEAIGKRPAVLPLLERVSEAAKSSGADGSLPSTPPPPEEEEDDLYRQSLEIISRYLREQATGSKDSKPLGEAGAAGRRALETLRRVGDGVQRNHETAFQGMLRKLDIKNEGDVKSFSRVMVHVFKDGVTNWGRIVTLISFGAFVAKHLKSVNQESFIEPLAETITDVLVRTKRDWLVKQRGWDGFVEFFHVQDLEGGIRNVLLAFAGVAGVGAGLAYLIR

SEQ ID NO: 40 - protein, 350 aa, Homo sapiens [sequence omitted]

SEQ ID NO: 41 - protein, 143 aa, Mus musculus
MKSQWCRPVAMDLGVYQLRHFSISFLSSLLGTENASVRLDNSSGASVVAIDNKIEQAMDLVKSHLMYAVREEVEVLKEQIKELIEKNSQLEQENNLLKTLASPEQLAQFQAQLQTGSPPATTQPQGTTQPPAQPASQGSGSTA

SEQ ID NO: 42 - protein, 144 aa, Homo sapiens
MKSQWCRPVAMDLGVYQLRHFSISFLSSLLGTENASVRLDNSSSGASVVAIDNKIEQAMDLVKSHLMYAVREEVEVLKEQIKELIEKNSQLEQENNLLKTLASPEQLAQFQAQLQTGSPPATTQPQGTTQPPAQPASQGSGPTA

SEQ ID NO: 43 - protein, 291 aa, Mus musculus
MVREQYTTVTEGTHIERPENQHIYKIGIYGWRKRCLYLFVLLLLAILVVNLALTIWILKVMWFSPIGMGHLHVTADGLRLEGESEFLFPLYAKEIRSRVDSSLLLQSTQNVTVSARNSEGEVTGRVKVGAQMVEVQSQHFQINSEDGKPLFSAEEQDVVVGTGRLRVTGPEGALFEHSVETPLVRADPFQDLRLESPTRSLSMDAPRGVHVKANAGKLEALSQMDIILQSSEGVLVLDAETVGLTKLKQGTQGPAGSSNGFYEICACPDGKLYLSMAGEVTTCEEHSHVCL

SEQ ID NO: 44 - protein, 291 aa, Homo sapiens
MVREQYTTATEGICIERPENQYVYKIGIYGWRKRCLYLFVLLLLIILVVNLALTIWILKVMWFSPAGMGHLCVTKDGLRLEGESEFLFPLYAKEIHSRVDSSLLLQSTQNVTVNARNSEGEVTGRLKVGPKMVEVQNQQFQINSNDGKPLFTVDEKEVVVGTDKLRVTGPEGALFEHSVETPLVRADPFQDLRLESPTRSLSMDAPRGVHIQAHAGKIEALSQMDILFHSSDGMLVLDAETVCLPKLVQGTWGPSGSSQSLYEICVCPDGKLYLSVAGVSTTCQEHSHICL

SEQ ID NO: 45 - protein, 228 aa, Homo sapiens
MWVQTRPSSASYKSWGPGTADTHTMRLSRRPETFLLAFVLLCTLLGLGCPLHCEICTAAGSRCHGQMKTCSSDKDTCVLLVGKATSKGKELVHTYKGCIRSQDCYSGVISTTMGPKDHMVTSSFCCQSDGCNSAFLSVPLTNLTENGLMCPACTASFRDKCMGPMTHCTGKENHCVSLSGHVQAGIFKPRFAMRGCATESMCFTKPGAEVPTGTNVLFLHHIECTHSP

SEQ ID NO: 46 - protein, 140 aa, Mus musculus
MASPLRSLLFLLAVLAVAWAATPKQGPRMLGAPEEADANEEGVRRALDFAVSEYNKGSNDAYHSRAIQVVRARKQLVAGVNYFLDVEMGRTTCTKSQTNLTDCPFHDQPHLMRKALCSFQIYSVPWKGTHSLTKFSCKNA

SEQ ID NO: 47 - protein, 146 aa, Homo sapiens
MAGPLRAPLLLLAILAVALAVSPAAGSSPGKPPRLVGGPMDASVEEEGVRRALDFAVGEYNKASNDMYHSRALQVVRARKQIVAGVNYFLDVELGRTTCTKTQPNLDNCPFHDQPHLKRKAFCSFQIYAVPWQGTMTLSKSTCQDA

SEQ ID NO: 48 - protein, 140 aa, Mus musculus
MASPLRSLLFLLAVLAVAWAATPKQGPRMLGAPEEADANKEGVRRALDFAVSEYNKGSNDAYHSRAIQVVRARKQLVAGVNYFLDVEMGRTTCTKSQTNLTDCPFHDQPHLMRKALCSFQIYSVPWKGTHSLTKFSCKNA

SEQ ID NO: 49 - skipped

SEQ ID NO: 50 - DNA, 2231 bp, Mus musculus
cggcgaggaagatggcggacgggaaggcgggagaggagaagccagagaagccgcagcgagccggagccgccggaggacctgaagaagaagcagaaaaacctgtgaaaactaagactgtttcttccagtaatggaggggaaagttccagtcgcagcgctgaaaagcgatcagctgaagacgaagctgcagacctcccaacaaagcctacaaagatgtccaagtttggatttgccataggtagtcagacggcacggaaagcgtccgccatctccattagactcggagcaagtaagcctaaggaaacagttccaactcttgctccaaaaactctgtcggtagcagcagctttcaatgaagatgaagatagtgagccagaagaaatgcctccagaagcaaagatgaggatgaagaacattggaagggacacaccaacatcagcgggaccaaactcctttaataaagggaagcatggcttttccgataaccagaagctgtgggagcgaaatataaaatctcatcttggaaacgtccacgaccaagacaattaagtgaagtgtctgagactggggtgtgggtgggtgcagttagaaggagcagtctcctttttgtaaagaatggtgtaagactagctttggagccgtttttctttcttctttttttcttttccttttttcttttttttttttttttttaagattgagtggtacactaataaatgagagtttgcaattagaggtaatttatgttttatatacagatttcaagacatttgctaattttgtagtttcacatgattagtttccaagggttacagataataaagaaatcacaagtggtacctttctaagaattgcatatttttttagacacaactattagcacattaagagggaagccaaaagttattgtctgcttcaaactggaagcagttcctctcctgacttctccctcgttacccgactgtccggctccctgcagcagccttaccgagagggagattggcttgagaagccagtgttactgttgtgactgttggctgagaggaagtttagatgaggttcaagtaaaccctttcctgcgggcatttcgttttgtttcgggccattctagctagtactgcttcgctctcagtgggtaccgatgcttgctctgtaaaaataaatttttttcgttaaaaattcttatatgaagaattgagtattatgactagcccactctaacggagtgtgtctccgttgaggaccttcagaggacactatttgctaccaaagtgaaccagtattctgaatgtgcttctcttggtttctgttctagttcctagaagcatttccaccagaacttgaggcaaaacataaggaagctgtttcttttaaagtacaaacaccaccaaaaatatcagtgtacatagtgctttatgtatttggctggcttttattttttaaaaggtttaaataacaaaaaaggaaaaaaaggtatagcattgtatgaaggcagatgaggcgacacacctgtgacatttgaagcatttgtgttactaacttactagatccggtttgaattgaattcacaaggtttcttacttgtccaactccaggcttattttacgtacatacaacctgaccagtggtttcaaaagcacatttagtgactgcaataggacagaaatgcttctctagacacatcttcattgcgagcgtctcatcgcatatgctcttagagttctgaatgtaggatgtttgttttgtatttttgtattgtatatgggctttttaaatgtgacagttaaatgcatctttaatagtcacagacaaaagagatgataaaatgtcctttgaaaatggcaatgttgagtttggaggcagcttcggggagtttcctgggtggcgatgattcactgggcagtttttaatgtagtgacttcaagaagcagcctgcggatattcctaacacttggttcactacagtttagtttacttcataagccccaagtaaaacgatggaaatgtacatagcactattagttcatacatgatttaagtatatcaagatacataaatttaacatttatgtctgcaaactgtccatttgtccattttactgtttgttgaaataacacctctcctacatattctttacttgactcgaataacaaattaacctgacgtcaagatggagagagacgactgagctgaatgtctttactaaaattacaataaattttgtcaaactcg

SEQ ID NO: 51 - DNA, 1066 bp, Homo sapiens
ggggaggccgcggcggggaaaatggcggacgggaaggcgggagacgagaagcctgaaaagtcgcagcgagctggagccgccggaggacctgaagaagaagcagaaaaacctgtgaaaactaagactgtttcttccagtaatggaggggaaagttccagtcgcagcgctgagaagcgatcagctgaagaagaagctgccgacctcccaacaaagcctacaaagatctccaagtttggatttgccataggtagtcagacgacaaagaaagcatcagccatatccatcaaacttggatcaagtaagcctaaagaaactgttccaactcttgctccaaaaactctttcagtagcagcagcttttaatgaagatgaagatagtgaaccagaggaaatgcctccagaagcaaagatgaggatgaagaatattggaagggatacaccaacatcagctggaccaaactccttcaataaaggaaagcatgggttttctgataaccagaagctgtgggagcgaaatataaaatctcatcttggaaatgtccatgaccaagacaattaaatgatgttttgaaattggggtgtggggtgggtgtaaagttaaaaggaacagtttccttttttaaagaatggtataagactatctttggagccgctttttttttctttttcatttttttaaaagattgagtggtacactaataaatgagagtttgaaattagaggtaatttatgttttatatacagatttcaagacatttgctaattttgtagtttcatgtgattagtttccaaaggttacagataataaagaaatcagaaatggtaccmattttaaraattgcycattttttttttagagcccamctattagccccmttaagakggdgyaaarchaawgtataaramtrcaagmatgwtarcctstctkarstcccttattacctaaacttgtctggctcccaggaacagccttatagagagagggagtattgtattgggaagaaaatgttactgaactattgactgaaagtaaatttagataaaatacaaaaaaaaaaaaaaaaaaa

SEQ ID NO: 52 - DNA, 773 bp, Mus musculus
attcggatccttgtagaaattgctggacgcttctgtacgtttcgcagtttgtcttcgctgatcggtgagacttcgagcagttaggatgccgcgtggaagccgaagccgcacttcccgggtgactcctccggccagccgggcccctcagatgagggctgctccccgaagagcacctgcagctcagcctccagcagcagctgcgccatctgcagttggctcacctgccgctgcgccccggcagccaggcctgatggcccagatggctaccaccgcggccggtgtggctgtgggctctgcagtgggacacaccctgggtcacgccatcactgggggcttcagcggaggtggcagtgctgagcccgcaaagcccgacatcacttaccaggagcctcagggagcccagctgcagaaccagcagtcttttggaccttgctctctagagatcaagcagtttctggagtgtgctcagaaccagagcgatgtcaagctctgtgagggcttcaacgaggtgctgcggcagtgcaggattgcaaatggtttaatgtaatcaagaaattcaagctgaagagatgtaacattggttctgtataattgatagtacaagtgtggacccttatatttctaacagttcattgctttggaatggccgtgaaagagatctaactgtggatgcttactgaggcttgtatgtagtgttggttactggggaagtgtttggcctccttggactgtgttgtgattgtgagttaaaaaaaataaattggttattctga

SEQ ID NO: 53 - DNA, 3057 bp, Mus musculus
aatgctggtggagacgggggagctggacaacacgtacatcctgtacaccgccgaccacggctaccacattggccagtttgggctggtgaagggcaagtctatgccgtatgaattcgacatcagagtcccgttctacgtgaggggccccaacgtggaagctggctctctgaacccccacattgtcctcaacattgacctgggcccccaccatactggatatcgctggactggacatccctgcagacatggacgggaagtctattctcaaactactggactcagagcggccagtgaaccggttccacttgaaaaagaagctgagggtcttggcgagactccttcctggtggagagaggcaaacttgctccacaagagggagggtgacaaagtgaatgcccaggaggagaacttcctgcccaagtaccagcgcgtgaaggacctgtgtcagcgagctgagtaccagacagcatgcgaacagctggggcagaagtggcagtgtgtggaggacgcttctgggacgctgaagctgcacaaatgtaaaggccccatgcggtttggtggcggcggtggcagcagagccctctccaacctggtgcccaagtatgacggccagagcagcgaggcctgcagctgtgacagtggcggtggaggggactacaaactgggcctggctggacgccgtaagctctttaagaaaaagtataagaccagctatgcccggaaccgctccatccgttccgtggccatcgaggtggacggtgagatataccacgtaggcttggatactgtgcctcagccccgcaaccttagcaagccgcactggccaggggcccctgaagaccaagatgacaaggatggtggcagtttcagtggtactggtggccttccagattattctgcccccaatcccatcaaagtgacccatcggtgctacatccttgagaatgacacagtccagtgcgacttggacctgtacaagtccctgcaggcttggaaagaccacaagctgcacatcgaccatgagatcgaaaccctgcagaacaaaattaagaaccttcgagaagtcaggggtcacctgaagaagaagcgaccggaagaatgtgactgccataraatcagttaccacagccaacacaaaggccgtctcaagcacaaaggctccagcctgcaccctttcaggaagggtctgcaggagaaggacaaggtgtggctgctgcgggacagaaacgcaagaagaaactgcgcaactgctcaaacggctgcagaacaacgatacgtgcagcatgcccggcctcacgtgctttacccacgacaaccaccactggcagacggcgccactctggacgctggggccgttctgcgcctgcaccagcgccaacaacaacacgtactggtgcttgaggaccataaatgagacccacaacttcctcttctgcgaatttgcaaccggcttcatagaatactttgacctcagtacagacccctaccagctgatgaacgcggtgaacacactggacagggacgtccttaaccaactgcacgtgcagctcatggagctaaggagctgtaaaggctacaagcagtgcaacccccggacccgcaacatggacctggggcttagagacggaggaagctatgaacaatacaggcagtttcagcgtcgaaaatggccagaaatgaagagaccttcttccaaatcactgggacagctatgggaaggttgggaaggctaagcggccatagagagaggaacctccaaaaccaggggcctcgtgtggctgcccaggccatgcaaaaaacacccgattcccagaagatgaatgttggaactgggagacctgacagaaggcagggctgctcttgggacaggaaatcctggaggacagcgcctggactttccgatgctcagtttctttgccctgctttgctctggatcaaacctcactggctgctctgggatgcgtgctcacacctggagtctctgctcaccctttcagaggctcacaaagacaaaggaactaatttccatggacacttcctccagagatggaaattgctgggattcgcccactcctcccctgcacccctcccccagtcatctagggaagcaagcttgttttaaccttcttactctttggagaaagcacggacatcccaggtgctgtcaacctcacagtcttgacaaagtctatagcacaaaacagtaccattcaccaggctggttgacctggctggctcagaagctgccttcaccacatacatgaccgctcacacgtaaccaacacagggaattgtaggggaatctcactaatatgaaatcccgcttttcaagagtcgcggtgtcaataaacgctgtggctaggatcaaggataatcccttgagctttcagacatttattcctgcccgggattcgttcctttgttatccatcccagaactgatgtttttctaaggtaccgaaaccccaagttgatgtgtgtcctgtgttttaatgacattgtatttgtaaagttttgtagtataagtaccatcttacagtgttcctgcccccagccaatgtctagctattggtatgaaaaaaaaaatctttgaatttttgtaaaaggcwawmwwrrramwwwwwwgctctttattattatcatttttttcttgaagcactcaagatgttgtcgaccttcagctgagggttggagaagcagagggtaatacattaatagtaaacctctccgattcaagggacctcataggtaagtctcaccagctttggggcatcgatctgtgaagcaaagcatccaactgcaccaaccctttgttcatccaggtagggtaacctggtcaatatggctgccagaaagcagctgtcagagcttcggaggtgggggtggaaatgtcagcccaacccgactcagcagagggggctcacatttctctaaaccagcagtttttcagctggggctccaacgacctttcacaggggtcctaaggtcatcagagagcacatttaatggtacagttcacacagtaacaatcacagctaagaagtagcacagtagcagc

SEQ ID NO: 54 - DNA, 732 bp, Mus musculus
ggactacaggttcacacaccatgattctgtttagaagacacaggaccttcctgctggccttcacactgctctgtaccctcctgggtctcgggtgccctctaacttgtgaggtgtgcaaaggctcggggcacacatgcagcgggaagatgaagacctgtgaagacggcaaagacgcatgcgtggtcctagtgagcgagtccagcacaaagggccgaaagtcagtgaacaccttcaaggcctgcatgaagtacaaagactgctactcaggcttcgtatcaaccaccatgactcccagtgactacatggtgtccaacgcccactgctgtcaaagtgacggttgcaacagtggctctgtgccccctcccttgaacaatcggacagagaatggcctgatgtgtccctcctgcattgcgcccttccaggagacctgtccaggaacccaggcagctcgctgcgtgggccgggaaacacactgcatctattttgctggcaatgtgcaggctggtattatccacacgaaatttgccacgaggggctgtgctacagagagcgcctgccacaccaaggcaggggctgaggtcccttcagccttctatctctacttcctgcgccgggcagactgccttccagccccttaccctcctggcaggggagagtgaagaaaccgagaagtatgtaacttgaggagccctgcagctttccagctgcctgtaaattaaacagatygacagt

SEQ ID NO: 55 - protein, 178 aa, Mus musculus
MADGKAGEEKPEKPQRAGAAGGPEEEAEKPVKTKTVSSSNGGESSSRSAEKRSAEDEAADLPTKPTKMSKFGFAIGSQTARKASAISIRLGASKPKETVPTLAPKTLSVAAAFNEDEDSEPEEMPPEAKMRMKNIGRDTPTSAGPNSFNKGKHGFSDNQKLWERNIKSHLGNVHDQDN

SEQ ID NO: 56 - protein, 178 aa, Mus musculus
MADGKAGDEKPEKSQRAGAAGGPEEEAEKPVKTKTVSSSNGGESSSRSAEKRSAEEEAADLPTKPTKISKFGFAIGSQTTKKASAISIKLGSSKPKETVPTLAPKTLSVAAAFNEDEDSEPEEMPPEAKMRMKNIGRDTPTSAGPNSFNKGKHGFSDNQKLWERNIKSHLGNVHDQDN

SEQ ID NO: 57 - protein, 153 aa, Mus musculus
MPRGSRSRTSRVTPPASRAPQMRAAPRRAPAAQPPAAAAPSAVGSPAAAPRQPGLMAQMATTAAGVAVGSAVGHTLGHAITGGFSGGGSAEPAKPDITYQEPQGAQLQNQQSFGPCSLEIKQFLECAQNQSDVKLCEGFNEVLRQCRIANGLM

SEQ ID NO: 58 - protein, 151 aa, Homo sapiens
MPRGSRSRTSRMAPPASRAPQMRAAPRPAPVAQPPAAAPPSAVGSSAAAPRQPGLMAQMATTAAGVAVGSAVGHTLGHAITGGFSGGSNAEPARPDITYQEPQGTQPAQQQQPCLYEIKQFLECAQNQGDIKLCEGFNEVLKQCRLANGLA

SEQ ID NO: 59 - DNA, 19 bp, Mus musculus
cgagcgctacattgtcgct

SEQ ID NO: 60 - DNA, 22 bp, Mus musculus
gtcttaaatttgcttgtgcccc

SEQ ID NO: 61 - DNA, 19 bp, Mus musculus
atcaacgggaagcccatca

SEQ ID NO: 62 - DNA, 20 bp, Mus musculus
gacatactcagcaccggcct

SEQ ID NO: 63 - protein, 823 aa, Mus musculus
MGFGSDLKNSQEAVLKLQDWELRLLETVKKFMALRIKSDKEYAYTLQNLCNQVDKESTVQVNYVSNVSKSWLLMIQQTEQLSRIMKTHAEDLNSGPLHRLTMMIKDKQQVKKSYVGIHQQIEAEMIKVTKTELEKLKSSYRQLIKEMNSAKEKYKEALAKGKETEKAKERYDKATMKLHMLHNQYVLALKGAQLHQSQYYDTTLPLLLDSVQKMQEEMIKALKGIFDDYSQITSLVTEEIVNVHKEIQMSVEQIDPSTEYNNFIDVHRTTAAKEQEIEFDTSLLEENENLQANEIMWNNLTADSLQVMLKTLAEELTQTQQMLLHKEAAVLELEKRIEESFETCEKKSDIVLLLGQKQALEELKQSVQQLRCSEAKCAAQKALLEQKVQENDGKEPPPVVNYEEDARSVTSMERKERLSKFESIRHSIAGIIKSPKSVLGSSTQVCDVISVGERPLAEHDWYHGAIPRIEAQELLKQQGDFLVRESHGKPGEYVLSVYSDGQRRHFIIQFVDNLYRFEGTGFSNIPQLIDHHFNTKQVITKKSGVVLLNPIPKDKKWVLNHEDVSLGELLGKGNFGEVYKGTLKDKTPVAIKTCKEDLPQELKIKFLQEAKILKQYDHPNIVKLIGVCTQRQPVYIIMELVPGGDFLTFLRKRKDELKLKQLVRFSLDVAAGMLYLESKNCIHRDLAARNCLVGENNTLKISDFGMSRQEDGGVYSSSGLKQIPIKWTAPEALNYGRYSSESDVWSFGILLWETFSLGVCPYPGMTNQQAREQVERGYRMSAPQNCPEEVFTIMMKCWDYKPENRPKFNDLHKELTVIKKMIT

SEQ ID NO: 64 - protein, 822 aa, Homo sapiens
MGFGSDLKNSHEAVLKLQDWELRLLETVKKFMALRIKSDKEYASTLQNLCNQVDKESTVQMNYVSNVSKSWLLMIQQTEQLSRIMKTHAEDLNSGPLHRLTMMIKDKQQVKKSYIGVHQQIEAEMIKVTKTELEKLKCSYRQLIKEMNSAKEKYKEALAKGKETEKAKERYDKATMKLHMLHNQYVLALKGAQLHQNQYYDITLPLLLDSLQKMQEEMIKALKGIFDEYSQITSLVTEEIVNVHKEIQMSVEQIDPSTEYNNFIDVHRTTAAKEQEIEFDTSLLEENENLQANEIMWNNLTAESLQVMLKTLAEELMQTQQMLLNKEEAVLELEKRIEESSETCEKKSDIVLLLSQKQALEELKQSVQQLRCTEAKFSAQKELLEQKVQENDGKEPPPVVNYEEDARSVTSMERKERLSKFESIRHSIAGIIRSPKSAVGSSALSDMISISEKPLAEQDWYHGAIPRIEAQELLKKQGDFLVRESHGKPGEYVLSVYSDGQRRHFIIQYVDNMYRFEGTGFSNIPQLIDHHYTTKQVITKKSGVVLLNPIPKDKKWILSHEDVILGELLGKGNFGEVYKGTLKDKTSVAVKTCKEDLPQELKIKFLQEAKILKQYDHPNIVKLIGVCTQRQPVYIIMELVSGGDFLTFLRRKKDELKLKQLVKFSLDAAAGMLYLESKNCIHRDLAARNCLVGENNVLKISDFGMSRQEDGGVYSSSGLKQIPIKWTAPEALNYGRYSSESDVWSFGILLWETFSLGVCPYPGMTNQQAREQVERGYRMSAPQHCPEDISKIMMKCWDYKPENRPKFSELQKELTIIKRKLT

SEQ ID NO: 65 - protein, 116 aa, Mus musculus
MRNSKTAISFFILVAVLGSQAGLIQEMEKEDRRYNPPIIHQGFQDTSSDCCFSYATQIPCKRFIYYFPTSGGCIKPGIIFISRRGTQVCADPSDRRVQRCLSTLKQGPRSGNKVIA

SEQ ID NO: 66 - protein, 120 aa, Homo sapiens
MKVSVAALSCLMLVTALGSQARVTKDAETEFMMSKLPLENPVLLDRFHATSADCCISYTPRSIPCSLLESYFETNSECSKPGVIFLTKKGRRFCANPSDKQVQVCMRMLKLDTRIKTRKN

SEQ ID NO: 67 - protein, 264 aa, Mus musculus
MESGFTSKDTYLSHFNPRDYLEKYYSFGSRHCAENEILRHLLKNLFKIFCLGAVKGELLIDIGSGPTIYQLLSACESFTEIIVSDYTDQNLWELQKWLKKEPGAFDWSPVVTYVCDLEGNRMKGPEKEEKLRRAIKQVLKCDVTQSQPLGGVSLPPADCLLSTLCLDAACPDLPAYRTALRNLGSLLKPGGFLVMVDALKSSYYMIGEQKFSSLPLGWETVRDAVEEAGYTIEQFEVISQNYSSTTSNNEGLFSLVGRKPGRSE

SEQ ID NO: 68 - protein, 264 aa, Homo sapiens
MESGFTSKDTYLSHFNPRDYLEKYYKFGSRHSAESQILKHLLKNLFKIFCLDGVKGDLLIDIGSGPTIYQLLSACESFKEIVVTDYSDQNLQELEKWLKKEPEAFDWSPVVTYVCDLEGNRVKGPEKEEKLRQAVKQVLKCDVTQSQPLGAVPLPPADCVLSTLCLDAACPDLPTYCRALRNLGSLLKPGGFLVIMDALKSSYYMIGEQKFSSLPLGREAVEAAVKEAGYTIEWFEVISQSYSSTMANNEGLFSLVARKLSRPL

SEQ ID NO: 69 - protein, 158 aa, Mus musculus [sequence omitted]

SEQ ID NO: 70 - protein, 158 aa, Homo sapiens [sequence omitted]

SEQ ID NO: 71 - protein, 109 aa, Homo sapiens
MLKLTPLPSKMKVSAALLCLLLMAATFSPQGLAQPDSVSIPITCCFNVINRKIPIQRLESYTRITNIQCPKEAVIFKTQRGKEVCADPKERWVRDSMKHLDQIFQNLKP

SEQ ID NO: 72 - protein, 462 aa, Mus musculus
MGPWTHSLRAVLLLVLLGVCTVRSDTPANCTYPDLLGTWVFQVGPRSSRSDINCSVMEATEEKVVVHLKKLDTAYDELGNSGHFTLIYNQGFEIVLNDYKWFAFFKYEVRGHTAISYCHETMTGWVHDVLGRNWACFVGKKVESHIEKVNMNAAHLGGLQERYSERLYTHNHNFVKAINTVQKSWTATAYKEYEKMSLRDLIRRSGHSQRIPRPKPAPMTDEIQQQILNLPESWDWRNVQGVNYVSPVRNQESCGSCYSFASMGMLEARIRILTNNSQTPILSPQEVVSCSPYAQGCDGGFPYLIAGKYAQDFGVVEESCFPYTAKDSPCKPRENCLRYYSSDYYYVGGFYGGCNEALMKLELVKHGPMAVAFEVHDDFLHYHSGIYHHTGLSDPFNPFELTNHAVLLVGYGRDPVTGIEYWIIKNSWGSNWGESGYFRIRRGTDECAIESIAVAAIPIPKL

SEQ ID NO: 73 - protein, 463 aa, Homo sapiens
MGAGPSLLLAALLLLLSGDGAVRCDTPANCTYLDLLGTWVFQVGSSGSQRDVNCSVMGPQEKKVVVYLQKLDTAYDDLGNSGHFTIIYNQGFEIVLNDYKWFAFFKYKEEGSKVTTYCNETMTGWVHDVLGRNWACFTGKKVGTASENVYVNTAHLKNSQEKYSNRLYKYDHNFVKAINAIQKSWTATTYMEYETLTLGDMIRRSGGHSRKIPRPKPAPLTAEIQQKILHLPTSWDWRNVHGINFVSPVRNQASCGSCYSFASMGMLEARIRILTNNSQTPILSPQEVVSCSQYAQGCEGGFPYLIAGKYAQDFGLVEEACFPYTGTDSPCKMKEDCFRYYSSEYHYVGGFYGGCNEALMKLELVHHGPMAVAFEVYDDFLHYKKGIYHHTGLRDPFNPFELTNHAVLLVGYGTDSASGMDYWIVKNSWGTGWGENGYFRIRRGTDECAIESIAVAATPIPKL

SEQ ID NO: 74 - DNA, 21 bp, Homo sapiens
cctcccctcttcaagggtcta

SEQ ID NO: 75 - DNA, 21 bp, Homo sapiens
aggagtaagacccctggacca

SEQ ID NO: 76 - DNA, 21 bp, Homo sapiens
cccaatgggaattcactcacc

SEQ ID NO: 77 - DNA, 22 bp, Homo sapiens
gcttccactctcgtaggctttc

SEQ ID NO: 78 - DNA, 25 bp, Homo sapiens
tcttaaccaccagatcattccttct

SEQ ID NO: 79 - DNA, 20 bp, Homo sapiens
ggatactgcgagcaaatggg

SEQ ID NO: 80 - protein, 347 aa, Homo sapiens
MSKRPSYAPPPTPAPATQMPSTPGFVGYNPYSHLAYNNYRLGGNPGTNSRVTASSGITIPKPPKPPDKPLMPYMRYSRKVWDQVKASNPDLKLWEIGKIIGGMWRDLTDEEKQEYLNEYEAEKIEYNESMKAYHNSPAYLAYINAKSRAEAALEEESRQRQSRMEKGEPYMSIQPAEDPDDYDDGFSMKHTATARFQRNHRLISEILSESVVPDVRSVVTTARMQVLKRQVQSLMVHQRKLEAELLQIEERHQEKKRKFLESTDSFNNELKRLCGLKVEVDMEKIAAEIAQAEEQARKRQEEREKEAAEQAERSQSSIVPEEEQAANKGEEKKDDENIPMETGNSPA

SEQ ID NO: 81 - protein, 197 aa, Homo sapiens
MWVQTRPSSASYKSWGPGTADTHTMRLSRRPETFLLAFVLLCTLLGLGCPLHCEICTAAGSRCHGQMKTCSSDKDTCVLLVGKATSKGKELVHTYKGCIRSQDCYSGVISTTMGPKDHMVTSSFCCQSDGCNSAFLSVPLTNLTENGLMCPACTASFRDKCMGPMTHCTGKENHCVSLSGHVQAGEWCLNLWKRKQN

SEQ ID NO: 82 - protein, 285 aa, Homo sapiens
MAVSESQLKKMVSKPQSDLLGLIQVMIVVFGDEPPVFSRPISASYPPYQATGPPNTSYMPGMPGGISPYPSGYPPNPSGYPGCPYPPGGPYPATTSSQYPSQPPVTTVGPSRDGTISEDTIRASLISAVSDKLRWRMKEEMDRAQAELNALKRTEEDLKKGHQKLEEMVTRLDQEVAEVDKNIELLKKKDEELSSALEKMENQSENNDIDEVIIPTAPLYKQILNLYAEENAIEDTIFYLGEALRRGVIDLDVFLKHVRLLSRKQFQLRALMQKARKTAGLSDLY

SEQ ID NO: 83 - DNA, 1946 bp, Homo sapiens
ctgcccgcggtgtctcagattcattcttaaggaactgagaacttaatcttccaaaatgtcaaaaagaccatcttatgccccacctcccaccccagctcctgcaacacaaatgcccagcacaccagggtttgtgggatacaatccatacagtcatctcgcctacaacaactacaggctgggagggaacccgggcaccaacagccgggtcacggcatcctctggtatcacgattccaaaaccccccaagccaccagataagccgctgatgccctacatgaggtacagcagaaaggtctgggaccaagtaaaggcttccaaccctgacctaaagttgtgggagattggcaagattattggtggcatgtggcgagatctcactgatgaagaaaaacaagaatatttaaacgaatacgaagcagaaaagatagagtacaatgaatctatgaaggcctatcataattcccccgcgtaccttgcttacataaatgcaaaaagtcgtgcagaagctgctttagaggaagaaagtcgacagagacaatctcgcatggagaaaggagaaccgtacatgagcattcagcctgctgaagatccagatgattatgatgatggcttttcaatgaagcatacagccaccgcccgtttccagagaaaccaccgcctcatcagtgaaattcttagtgagagtgtggtgccagacgttcggtcagttgtcacaacagctagaatgcaggtcctcaaacggcaggtccagtccttaatggttcatcagcgaaaactagaagctgaacttcttcaaatagaggaacgacaccaggagaagaagaggaaattcctggaaagcacagattcatttaacaatgaacttaaaaggttgtgcggtctgaaagtagaagtggatatggagaaaattgcagctgagattgcacaggcagaggaacaggcccgcaaaaggcaggaggaaagggagaaggaggccgcagagcaagctgagcgcagtcagagcagcatcgttcctgaggaagaacaagcagctaacaaaggcgaggagaagaaagacgacgagaacattccgatggagacaggaaacagcccagcatgactttaaacaaagtgtctaaaagaccaaccttatattatttctacaactgctttctcctcgtttaatccatagcactacagactaactctcattcactgtccaacagaaataaaatgcaagtcacgtgaaattttatattttctactagtgacatatttttaaaagttaagcaaaacaagtgttttaacaatatgttttatttaattcaacatatcctaaataaaattatttcaacacagaaggaacatttttaaaatcagctttcctttttttaaacagtctttatacacatctcaattcagaagctaaattttcatcggaagtatttgatctgcatttatttaataaatttcattcatttacagctaaaaagtagattttcatacccaaattgttccaacattcttaaaagttctttagtcgcactggttcatttcaggggtttaataaccacatgtggctagtggcttctatattggacagcacagctgtatactgtcagcataactagagtaaaatttcatcttggaactgacagtgacaaatgtgtgattacagaggagacacaccttgaagaaacaacagagagccaacagaatggtgaagaaggcacgtctactcctgaggacaaggagagtgggcaggagggggtcgacagtatggcagaggaaggaaccagtgatagtaacactggctcggagagcaacagtgcaacagtggaggagccaccaacagatcccataccagaagatgagaaaaaagaataagtgttgccttgttttgtgtgttctaaatactttttttaatgaaaaaatgttttttggttttaatggtgttacgtggtttg

SEQ ID NO: 84 - DNA, 932 bp, Homo sapiens
ccggccgtgggaaaggtgaatgtgggtccagacccgcccctcctcagcttcctataaaagctggggaccaggtactgctgatacacacaccatgaggctctccaggagaccagagacctttctgctggcctttgtgttgctctgcaccctcctgggtcttgggtgcccactacactgcgaaatatgtacggcggcggggagcaggtgccatggccaaatgaagacctgcagcagtgacaaggacacatgtgtgctcctggtcgggaaggctacttcaaagggcaaggagttggtgcacacctacaagggctgcatcaggtcccaggactgctactccggcgttatatccaccaccatgggccccaaggaccacatggtaaccagctccttctgctgccagagcgacggctgcaacagtgcctttttgtctgttcccttgaccaatcttactgagaatggcctgatgtgccccgcctgcactgcgagcttcagggacaaatgcatggggcccatgacccactgtactggaaaggaaaaccactgtgtctccttatctggacacgtgcaggctggtgagtggtgcctgaatctctggaaaaggaaacagaactagaggtccaaacttctaggttcgatgggaggagagggttccagagaagtgggtgaggatgtgttctgggattatgaggaagaaggggctgaggtccctgattccagtctgaaatctccctttcaggtattttcaaacccagatttgctatgcggggctgtgctacagagagtatgtgctttaccaagcctggtgctgaagtacccacaggcaccaatgtcctcttcctccatcatatagagtgcactcactccccctgaaaagctatctgaacaaaggaagataatgtagtgtgaagtccccatttgtcctcagcctgtaa

SEQ ID NO: 85 - DNA, 3626 bp, Mus musculus
ggccattaccaatcgcgacccgcgcacacacggcccgggcggcgggcgaagcgggctcccggggcgctgggcgcagggcgcggggcaagccccagcagcgtgtctgcagcggggcgcggcgggcgctccagctccgggatctttctccctccgtcacctccctcgcgtctagggaggtcgtggcactccctgaggagcgcggctgctcggagggcggatcctaggctgtctatatgtgatacagcacgcactgcattgaagtggcagcacaagaatcggagattattttcatctctgagaacagaggcgtgagagccggcatgaatggtcatatgtctaaccgctccagtgggtatggagtctacccttctcaactgaatggttacggatcttcaccaccctattcccagatggacagagaacacagctcaagaacaagtgcaaaggccctttatgaacaaaggaagaactatgcccgagacagtgtcagcagtgtgtcggacgtgtcccagtaccgcgtggaacacttgaccaccttcgtgctggatcggaaagatgcaatgatcactgtcgaggacggaataagaaagctgaagttgctggatgccaagggcaaagtgtggactcaagatatgattctccaagtggatgaccgagctgtgagcctgattgacttagagtcaaagaatgaattggagaattttcctctaaacacaatctcgcattgtcaagcagtggtacatgcatgcagctatgactccattctcgccttggtatgcaaagagccaacgcagagcaagccagaccttcaccttttccagtgtgatgaggttaaggcaaacctaattagtgaagatatcgaaagtgcaatcagtgacagtaaaggtgggaaacagaagaggcggccggaggccctgaggatgattgccaaagcagatcctggcatccctcctcctcccagagctcctgcccctgtgccaccagggactgtcacacaggtggacgttaggagtcgcgtagcagcctggtctgcctgggcagctgaccagggcgacttcgagaagccccggcagtaccacgagcaagaagagacgcccgagatgatggcagcccggatcgacagggatgtgcaaatcttaaaccatattttggatgacattgaattttttatcaccaaactccaaaaagccgccgaagcgttttctgagctttctaaaaggaagaaaagtaagaaaagtaaaaggaaaggacctggagagggcgttttaacactgagggcaaaaccgccacctcctgatgaatttgttgactgtttccagaagtttaaacatggattcaaccttctggccaagttgaagtcccatatccagaacccgagtgcttcagatctggttcattttttgtttactccactaaatatggtggtccaggcaacaggtggccccgaactggccagttcggtactcagcccactgttgacaaaagacacagttgatttcttaaactacacagccactgcggaggaacggaagctgtggatgtcactgggagatagttgggtgaaagtgagagcagagtggccgaaagaacagttcatcccaccttacgtcccgaggttccgcaacggctgggagcccccgatgctgaacttcatgggcgcgcccacagagcaagacatgtatcaactggccgagtccgtggccaacgcagaacaccagcgcaaacaggacagcaagaggctgtccacagagcattccaatgtgtccgactatcctccagccgacggatatgcgtacagtagcagcatgtaccacagaggaccacatgcagaccacggggaggctgccatgcctttcaagtcaactcctaatcaccaagtagataggaattatgacgcagtcaaaacacaacccaagaaatacgccaaatccaagtacgactttgtggcgaggaacagcagcgagctctcggttatgaaagatgatgtcttagagatactcgacgatcgaaggcagtggtggaaagtccggaatgccagtggagactctgggtttgtgccaaataacattctggatatcatgagaactccggaatctggagtggggcgcgctgaccccccatacacacataccatacagaaacaaaggacggaatacggcctgagatcagctgacactccttctgccccatcaccccctccaacgccagcacccgttccggtcccccttccaccttctgtaccagcacccgtttctgtgcccaaggtcccagccaatgtcacccgccagaacagcagctccagtgacagtgggggcagcattgtgcgggacagccagagatacaaacaactcccagtggaccgaaggaagtcccagatggaagaggttcaggatgagctcttccagaggctgaccatcgggcgcagtgctgcacagaggaagttccacgtgccacggcagaacgttccagtgatcaatatcacttatgactcctcaccggaagaagtaaagacttggctgcagtcaaagggattcaaccccgtgactgtcaatagcctcggggtgttgaacggagcacaactcttttctctcaacaaagacgaactgaggtctgtctgcccggaaggtgccagagtctttaaccaaatcactgttcagaaagctgctttggaggacagtaatggaagctccgagttacaagagatcatgcggagacggcaggagaagatcagcgccgctgcgagcgactcgggagtggagtctttcgatgaagggagcagccactgagtccatgaacttccttattcttggtgtggtcattgaacagtgatggacatgctttgttttaagaagccttgaagggaatgtcaaagctgtcgtcttggtatatgtaatttatcgccatataaagaaacagtatatgcctgagtaagcagaggacccgctgcttctgtgcacattagtttgattaaaactgagaagcgggtaggtgagatggctcagcaagtaaaggtgcttgctgccaagcccaatgacccaagttcgagtccctgggtctacatggtaggagagagctggcttctgcaagttgtcctctgatcaccacacataaataaataacaaatgtaatttacaaacttttaaaagaaaatgtaatttaaaaagccagacgttctagactgttctgggcttgggaaatattttttttcactttcctaagatgtactttcctttgctacattaattattgcagccttgttcgatgatctaagtggggatatttgacaatggcagatttattcattgcaacaaggaaagacacagccattgatgaaaaaaaaaagaaagtctcagctttcagtgactgggatacctgctgtccagggaggaggctcagttagactaccctctgcttacttgaggtctgacatgcccaatgagagtgtatttagctttatttaaagttcttaatgccaacagttttaaaaatcacatttaaatgaactgtacaaggtagccagaccttgaatgtatgaatagactatataatatgtcccgagaaactttgttactctcagctctgttgattgcgaaatcttgcatagattatgctttgatttagtttct

SEQ ID NO: 86 - DNA, 3832 bp, Homo sapiens
cgcgaggccggcgctgtgctcgcctcggagatcgctgctctttagctgggtgcagaaggcggctccgcggctcgcggacgactggctgggcgcgaatcagattggggggctttctcccggtcccctcccacctcgtctgggctcgcggcgtctccggggaaagccgtggccccgagggcggatccgagaacacacaagtgaaagacacaatgaatggtcatatttctaatcatcccagtagttttggaatgtacccatctcagatgaatggctacggatcatcacctaccttttcccagacggacagagaacatggttcaaaaacaagtgcaaaggccctttatgaacaaaggaagaattatgcacgggacagtgtcagcagtgtgtcagatatatctcaataccgtgttgaacacttgactacctttgtcctggatcggaaagatgctatgatcactgttgatgatggaataaggaaattgaaattgcttgatgccaagggcaaagtgtggactcaagatatgattcttcaagtggatgacagagctgtgagcctgattgatttagaatcaaagaatgaactggagaattttcctttaaacacaatccagcactgccaagctgtgatgcattcatgcagctatgattcagttcttgcactggtgtgcaaagagccaacccagaacaagccagatcttcatctcttccagtgtgatgaggttaaggcaaacctaattagtgaagatattgaaagtgcaatcagtgacagtaaaggagggaaacagaagaggcggcccgacgccctgaggatgatttccaatgcagaccctagtataccgcctccacccagagctcctgcccctgcgccccctgggaccgtcacccaggtggatgttagaagtcgagtggcagcctggtctgcatgggcagccgaccaaggggactttgagaaaccaaggcagtatcatgagcaggaagaaacacctgagatgatggcagcccgcattgacagagatgtgcaaatcttaaaccacattttggatgacattgaattttttatcacaaaactccaaaaagcagcagaagcattttctgagctttctaaaaggaagaaaaacaagaaaggtaaaaggaaaggaccaggagagggtgttttaacgctgcgggcaaaacctccacctcctgatgaatttcttgactgtttccaaaagtttaaacacggatttaaccttctggccaaactgaagtctcatattcagaatcctagtgctgcagatttggttcactttttgtttactccattaaatatggtggtgcaggcaacaggaggtcctgaactagccagttcagtacttagtcccctattgaataaggacacaattgatttcttaaattatactgtcaatggtgatgaacggcagctgtggatgtcattgggaggaacttggatgaaagccagagcagagtggccaaaagaacagtttattccaccatatgttccacgattccgcaatggctgggagcccccaatgctgaactttatgggagccacaatggaacaagatctttatcaactggcagaatctgtggcaaatgtagcagaacatcagcgcaaacaggaaataaaaagattatccacagagcattccagtgtatcagagtatcatccagccgatggctatgcgttcagtagcaacatttacacaagaggatcccacctggaccaaggggaagctgctgttgcttttaagccaacttctaatcgccatatagatagaaattatgaaccactcaaaacacaacccaagaaatatgccaaatccaagtatgactttgtagcaaggaacaacagtgagctctcggttctaaaggatgatattttagagatacttgatgatcggaagcaatggtggaaagttcgaaatgcaagtggagactctggatttgtgccaaataacattttggatattgtgagacctccagaatctggattggggcgtgctgatccaccttatactcatactatacagaaacaaaggatggagtatggcccaagaccagctgatactccccctgctccatcacctcctccaacaccagctcctgttcctgttccccttcccccttccactccagcacctgttcctgtgtcaaaggtcccagcaaatataacacgtcaaaacagcagctccagtgacagtggtggcagtatcgtgcgagacagccagagacacaaacaacttccggtggaccgaaggaaatctcagatggaggaagtgcaagatgaactcatccacagactgaccattggtcggagtgccgctcagaagaaattccatgtgccacggcagaacgtgccagttatcaatatcacttacgactccacaccagaggatgtgaagacgtggttacagtcaaagggattcaaccctgtgactgtcaatagtcttggagtattaaatggtgcacaacttttctctctcaataaggatgaactgaggacagtctgccctgaaggggcgagagtctatagccaaatcactgtacaaaaagctgcattggaggatagcagtggcagctccgagttacaagaaattatgagaagacgacaggaaaaaatcagtgctgccgctagtgattcaggagtggaatcttttgatgaaggaagcagtcactaatttgtttgtttgtatttaaactccattgtttttggcattattccaacatgctttgttttaagaagccttgaagggaatgtcagattcatttttcttgatgtaatttatcaccataaaaaaaaaacccatgcaaacctgagtgagcacaggatttgcttctaggcccattatttttattaaaactgaaaaaatttaaactgaattttttgaccttggaaaatatttttcttactttaccaaggtgaagtttccttaattagactaattattttatccccatcccagggtataaacaggaattgttttgatagtggtggagttattcactgcaacaaagcaacaatgttgtccatgattcaaaatctaagcagtttcgattttgcctgtgaatatggtgtctgtcattcagggcatagctcactgtaggctagcctctgcttacttaagtctcttctctgacatactcaatggaagaatatttagatttatttaaagttcttaatgccaacagtttaaaaaaaaattaaaacatttgaatgaactgtaaagtacagccataccttggacatgcaaatataaatctatggagcattctcaagacagtttgtcatggctctgttgattgcaactccttgtatagcttgtattttgatttagtttatattctgcttattatgtatactgtgttcttatatatgagaaagcacaaatgcgaaagaggtcatgtcttctcaaaatctagcaaaggaagtagtctgcattggtgtgcattacagtattttgcttaatgaaagcctcagttctgaatgttgatatgagtagttaaaaggaagtggggccattttatgtgtttatctgtgtcaagtatttctggtaataagaagcacttaatttacacatattttaatcctgtgaaagattccacatagagaaaagaaagatacctaaccttcaacaaatgttatttttggaaacacaatttttgtcattaaatgttatattatttcacatatataaaacagatgttatgtaagaatgttgtatattttaacataaatcatttagagaaattatctagattcattaattttcatagtgcctttttcacatgagtcagctggaaagtctgcaataaacagtatttgctgtctgtt